(19) United States
(12) Patent Application Publication
(10) Pub. No.: US 2024/0188432 A1
(43) Pub. Date: Jun. 6, 2024

(54) PLURALITY OF HOST MATERIALS AND ORGANIC ELECTROLUMINESCENT DEVICE COMPRISING THE SAME

(71) Applicant: ROHM AND HAAS ELECTRONIC MATERIALS KOREA LTD., Chungcheongnam-do (KR)

(72) Inventors: Bitnari KIM, Gyeonggi-do (KR); Su-Hyun LEE, Gyeonggi-do (KR); Hyo-Soon PARK, Gyeonggi-do (KR); Tae-Jun HAN, Gyeonggi-do (KR); Jeong-Eun YANG, Gyeonggi-do (KR); Sang-Hee CHO, Gyeonggi-do (KR); Doo-Hyeon MOON, Gyeonggi-do (KR)

(21) Appl. No.: 18/430,070

(22) Filed: Feb. 1, 2024

Related U.S. Application Data
(63) Continuation of application No. 16/972,948, filed on Dec. 7, 2020, now abandoned.

(30) Foreign Application Priority Data
Jun. 22, 2018 (KR) 10-2018-0072267
Apr. 18, 2019 (KR) 10-2019-0045304

(51) Int. Cl. H10K 85/60; H10K 50/11; H10K 50/12; H10K 50/15; H10K 50/17; H10K 101/00; H10K 101/10

(52) U.S. Cl. CPC H10K 85/6572; H10K 85/622; H10K 85/654; H10K 85/6574; H10K 85/6576; H10K 50/11; H10K 50/12; H10K 50/15; H10K 50/171; H10K 2101/10; H10K 2101/90

(57) ABSTRACT

The present disclosure relates to a plurality of host materials comprising a first host material having a compound represented by formula 1, and a second host material having a compound represented by formula 2, and an organic electroluminescent device comprising the same. By comprising a specific combination of compounds of the present disclosure as host materials, it is possible to provide an organic electroluminescent device having higher luminous efficiency and/or longer lifespan properties as compared with a conventional organic electroluminescent device.

### CLAIM OF BENEFIT OF PRIOR APPLICATION

This application claims priority under 35 U.S.C. § 120 from U.S. patent application Ser. No. 16/972,948, filed Dec. 7, 2020, which is the National Stage Entry of PCT/KR2019/005686, filed May 13, 2019, each of which is incorporated by reference herein in its entirety.

### TECHNICAL FIELD

The present disclosure relates to a plurality of host materials and an organic electroluminescent device comprising the same.

### BACKGROUND ART

In 1987, Tang et al. of Eastman Kodak first developed a small molecule green organic electroluminescent device (OLED) of TPD/Alq3 bilayer consisting of a light-emitting layer and a charge transport layer. Since then, the research on an OLED has been rapidly carried out, and it has been commercialized. At present, OLEDs primarily use phosphorescent materials having excellent luminous efficiency in panel implementation. In addition, an OLED having high luminous efficiency and/or long lifespan is required for prolonged use and high resolution of a display.

Korean Patent Appl. Laid-Open No. 2017-0022865 discloses a benzoxazole derivative compound for improving the performance of an OLED. However, the development of materials for improving the performance of an OLED is still required.

### DISCLOSURE OF THE INVENTION

### Problems to be Solved

The objective of the present disclosure is to provide a plurality of host materials comprising a specific combination of compounds, suitable for producing an organic electroluminescent device having higher luminous efficiency and/or longer lifespan properties.

### Solution to Problems

The present inventors found that the above objective can be achieved by a plurality of host materials comprising a first host material and a second host material, wherein the first host material comprises a compound represented by the following formula 1, and the second host material comprises a compound represented by the following formula 2:

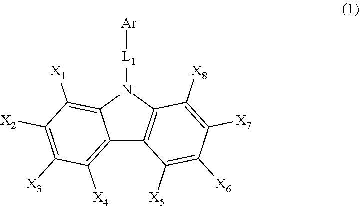

wherein,
Ar represents a substituted or unsubstituted (C6-C30)aryl, or a substituted or unsubstituted (3- to 30-membered)heteroaryl containing at least one of nitrogen, oxygen, and sulfur;
L₁ represents a single bond, a substituted or unsubstituted (C6-C30)arylene, or a substituted or unsubstituted (3- to 30-membered)heteroarylene;
X₁ to X₈, each independently, represent hydrogen, deuterium, a halogen, a cyano, a carboxyl, a nitro, a hydroxyl, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, a substituted or unsubstituted (3- to 30-membered)heteroaryl, —NR₅R₆, or —SiR₇R₈R₉; or adjacent ones of X₁ to X₈ may be linked to each other to form a ring(s); with the proviso that at least one pair of X₁ and X₂, X₂ and X₃, X₃ and X₄, X₄ and X₅, X₅ and X₆, X₆ and X₇, and X₇ and X₈ may be linked to each other to form a ring(s), in which the ring has 1 to 5 monocyclic rings;
R₅ to R₉, each independently, represent hydrogen, deuterium, a halogen, a cyano, a carboxyl, a nitro, a hydroxyl, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, or a substituted or unsubstituted (3- to 30-membered)heteroaryl; or adjacent ones of R₅ to R₉ may be linked to each other to form a ring(s); and

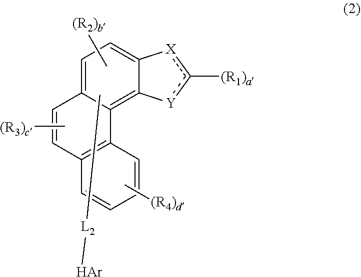

wherein
X represents —N═, —NR₁₀—, —O—, or —S—;
Y represents —N═, —NR₁₁—, —O—, or —S—, with the proviso that when X represents —N═, Y represents —NR₁₁—, —O—, or —S—, and when X represents —NR₁₀—, Y represents —N═, —O—, or —S—;
HAr represents a substituted or unsubstituted (3- to 30-membered)heteroaryl containing a nitrogen atom(s);
L₂ represents a single bond, a substituted or unsubstituted (C6-C30)arylene, or a substituted or unsubstituted (3- to 30-membered)heteroarylene;
R₁ represents a substituted or unsubstituted (C6-C30)aryl, or a substituted or unsubstituted (3- to 30-membered)heteroaryl;
R₂ to R₄, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or adjacent ones of R₂ to R₄ may be linked to each other to form a ring(s);
R₁₀ and R₁₁, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino;
a′ represents an integer of 1; b′ and c′, each independently, represent an integer of 1 or 2; d′ represents an integer of 1 to 4; where if b′, c′, and d′, each independently, are an integer of 2 or more, each of R₂ to R₄ may be the same or different.

### Effects of the Invention

By comprising a specific combination of compounds of the present disclosure as host materials, it is possible to provide an organic electroluminescent device having higher luminous efficiency and/or longer lifespan properties as compared with the conventional organic electroluminescent device, and manufacture a display system or a light system using the same.

### EMBODIMENTS OF THE INVENTION

Hereinafter, the present disclosure will be described in detail. However, the following description is intended to explain the disclosure, and is not meant in any way to restrict the scope of the disclosure.

The term “organic electroluminescent material” in the present disclosure means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material (containing host and dopant materials), an electron buffer material, a hole blocking material, an electron transport material, an electron injection material, etc.

The term “a plurality of organic electroluminescent materials” in the present disclosure means an organic electroluminescent material as a combination of at least two compounds, which may be comprised in any layer constituting an organic electroluminescent device. It may mean both a material before being comprised in an organic electroluminescent device (for example, before vapor deposition) and a material after being comprised in an organic electroluminescent device (for example, after vapor deposition). For example, a plurality of organic electroluminescent materials may be a combination of at least two compounds which may be comprised in at least one of a hole injection layer, a hole transport layer, a hole auxiliary layer, a light-emitting auxiliary layer, an electron blocking layer, a light-emitting layer, an electron buffer layer, a hole blocking layer, an electron transport layer, and an electron injection layer. At least two compounds may be comprised in the same layer or different layers by means of the methods used in the art, for example, may be mixture-evaporated or co-evaporated, or may be individually evaporated.

The term “a plurality of host materials” in the present disclosure means an organic electroluminescent material as a combination of at least two host materials. It may mean both a material before being comprised in an organic electroluminescent device (for example, before vapor deposition) and a material after being comprised in an organic electroluminescent device (for example, after vapor deposition). A plurality of host materials of the present disclosure may be comprised in any light-emitting layer constituting an organic electroluminescent device. At least two compounds comprised in a plurality of host materials may be comprised together in one light-emitting layer or may respectively be comprised in different light-emitting layers. If at least two host materials are comprised in one layer, for example, they may be mixture-evaporated to form a layer, or may be separately co-evaporated at the same time to form a layer.

Herein, the term “(C1-C30)alkyl” is meant to be a linear or branched alkyl having 1 to 30 carbon atoms constituting the chain, in which the number of carbon atoms is preferably 1 to 10, and more preferably 1 to 6. The above alkyl may include methyl, ethyl, n-propyl, iso-propyl, n-butyl, iso-butyl, tert-butyl, etc. The term “(C3-C30)cycloalkyl” is meant to be a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, etc. The term “(3- to 7-membered)heterocycloalkyl” is meant to be a cycloalkyl having 3 to 7 ring backbone atoms, and including at least one heteroatom selected from the group consisting of B, N, O, S, Si, and P, and preferably the group consisting of O, S, and N. The above heterocycloalkyl may include tetrahydrofuran, pyrrolidine, thiolan, tetrahydropyran, etc. The term “(C6-C30)aryl” or “(C6-C30)arylene” is meant to be a monocyclic or fused ring radical derived from an aromatic hydrocarbon having 6 to 30 ring backbone carbon atoms, in which the number of the ring backbone carbon atoms is preferably 6 to 20, more preferably 6 to 15. The above aryl or arylene may be partially saturated, and may comprise a spiro structure. The above aryl may include phenyl, biphenyl, terphenyl, naphthyl, binaphthyl, phenylnaphthyl, naphthylphenyl, fluorenyl, phenylfluorenyl, benzofluorenyl, dibenzofluorenyl, phenanthrenyl, phenylphenanthrenyl, anthracenyl, indenyl, triphenylenyl, pyrenyl, tetracenyl, perylenyl, chrysenyl, naphthacenyl, fluoranthenyl, spirobifluorenyl, spiro[fluorene-benzofluorene]yl, etc. The term “(3- to 50-membered)heteroaryl” or “(3- to 30-membered)heteroarylene” is an aryl having 3 to 50, or 3 to 30 ring backbone atoms, in which the number of the ring backbone carbon atoms is preferably 3 to 30, more preferably 5 to 20, and including at least one, preferably 1 to 4 heteroatoms selected from the group consisting of B, N, O, S, Si, and P. The above heteroaryl(ene) may be a monocyclic ring, or a fused ring condensed with at least one benzene ring; may be partially saturated; may be one formed by linking at least one heteroaryl or aryl group to a heteroaryl group via a single bond(s); and may comprise a spiro structure. The above heteroaryl may include a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl such as benzofuranyl, benzothiophenyl, isobenzofuranyl, dibenzofuranyl, benzonaphthofuranyl, dibenzothiophenyl, benzonaphthothiophenyl, benzimidazolyl, benzothiazolyl, benzoisothiazolyl, benzoisoxazolyl, benzoxazolyl, isoindolyl, indolyl, indazolyl, benzothiadiazolyl, quinolyl, isoquinolyl, cinnolinyl, quinazolinyl, quinoxalinyl, naphthyridinyl, carbazolyl, benzocarbazolyl, phenoxazinyl, phenanthridinyl, phenanthro-oxazolyl, benzodioxolyl, etc. Furthermore, “halogen” includes F, Cl, Br, and I.

Herein, “substituted” in the expression “substituted or unsubstituted” means that a hydrogen atom in a certain functional group is replaced with another atom or another functional group, i.e., a substituent. The substituents of the substituted alkyl, the substituted cycloalkyl, the substituted cycloalkenyl, the substituted heterocycloalkyl, the substituted aryl, the substituted arylene, the substituted heteroaryl, the substituted heteroarylene, the substituted alkoxy, the substituted trialkylsilyl, the substituted dialkylarylsilyl, the substituted alkyldiarylsilyl, the substituted triarylsilyl, the substituted mono- or di-alkylamino, the substituted mono- or di-arylamino, or the substituted alkylarylamino in Ar, L₁, HAr, L₂, X₁ to X₈, X₁₁ to X₃₃, and R₁ to R₁₅, each independently, are at least one selected from the group consisting of deuterium; a halogen; a cyano; a carboxyl; a nitro; a hydroxyl; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 50-membered)heteroaryl unsubstituted or substituted with at least one of a (C1-C30)alkyl(s), a (C6-C30)aryl(s), and a di(C6-C30)arylamino(s); a (C6-C30)aryl unsubstituted or substituted with at least one of a cyano(s), a (C1-C30)alkyl(s), a (3- to 50-membered)heteroaryl(s), a di(C6-C30)arylamino(s), and a tri(C6-C30)arylsilyl(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; an amino; a mono- or di-(C1-C30)alkylamino; a mono- or di-(C6-C30)arylamino; a (C1-C30)alkyl(C6-C30)arylamino; a (C1-C30)alkylcarbonyl; a (C1-C30)alkoxycarbonyl; a (C6-C30)arylcarbonyl; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl. According to one embodiment of the present disclosure, the substituents, each independently, are at least one selected from the group consisting of a (C1-C20)alkyl; a (C6-C25)aryl unsubstitued or substituted with at least one of a (C1-C20)alkyl(s), a (3- to 30-membered)heteroaryl(s), and a di(C6-C25)arylamino(s); a (3- to 30-membered)heteroaryl unsubstituted or substituted with at least one of a (C1-C20)alkyl(s) and a (C6-C25)aryl(s); and a di(C6-C20)arylamino. According to another embodiment of the present disclosure, the substituents, each independently, are at least one selected from the group consisting of a (C1-C10)alkyl; a (C6-C20)aryl unsubstitued or substituted with at least one of a (C1-C10)alkyl(s) and a di(C6-C18)arylamino(s); a (5- to 25-membered)heteroaryl unsubstituted or substituted with a (C6-C18)aryl(s); and a di(C6-C18)arylamino. For example, the substituents, each independently, are at least one methyl; tert-butyl; phenyl unsubstituted or substituted with at least one of pyridinyl, diphenyltriazinyl, phenylquinoxalinyl, phenylquinazolinyl, biphenylquinazolinyl, dibenzofuranyl, dibenzothiophenyl and diphenylamino; naphthyl unsubstituted or substituted with at least one diphenyltriazinyl; biphenyl; naphthylphenyl; terphenyl; dimethylfluorenyl; phenylfluorenyl; diphenylfluorenyl; dimethylbenzofluorenyl; phenanthrenyl; triphenylenyl; pyridinyl; triazinyl substituted with at least one of phenyl and naphthyl; indolyl substituted with at least one phenyl; benzoimidazolyl substituted with at least one phenyl; quinolyl; quinazolinyl substituted with at least one of phenyl and biphenyl; quinoxalinyl substituted with at least one phenyl; carbazolyl unsubstituted or substituted with at least one phenyl; dibenzofuranyl; dibenzothiophenyl; benzonaphthothiophenyl; benzocarbazolyl unsubstituted or substituted with at least one phenyl; dibenzocarbazolyl; benzophenanthrothiophenyl; diphenylamino; dimethylfluorenylphenylamino; and a substituted or unsubstituted (16- to 33-membered)heteroaryl containing at least one of nitrogen, oxygen, and sulfur.

In the formulas of the present disclosure, “adjacent ones may be linked to each other to form a ring(s)” means that at least two adjacent substituents are linked to or fused with each other to form a substituted or unsubstituted mono- or polycyclic (3- to 30-membered), preferably (3- to 26-membered), alicyclic or aromatic ring, or the combination thereof. Also, the formed ring may contain at least one heteroatom selected from B, N, O, S, Si, and P, preferably at least one heteroatom selected from N, O, and S.

Herein, the heteroaryl, the heteroarylene, and the heterocycloalkyl, each independently, may contain at least one heteroatom selected from B, N, O, S, Si, and P. Also, the heteroatom may be bonded to at least one selected from the group consisting of hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, and a substituted or unsubstituted (C1-C30)alkyl(C6-C30)arylamino.

In formula 1, Ar represents a substituted or unsubstituted (C6-C30)aryl, or a substituted or unsubstituted (3- to 30-membered)heteroaryl containing at least one of nitrogen, oxygen, and sulfur. According to one embodiment of the present disclosure, Ar represents a substituted or unsubstituted (C6-C25)aryl, or a substituted or unsubstituted (5- to 25-membered)heteroaryl containing at least one of nitrogen, oxygen, and sulfur. According to another embodiment of the present disclosure, Ar represents a (C6-C18)aryl unsubstituted or substituted with a (C1-C30)alkyl(s), or a (5- to 20-membered)heteroaryl unsubstituted or substituted with a (C6-C18)aryl(s) and containing nitrogen, oxygen or sulfur. Specifically, Ar represents a substituted or unsubstituted phenyl, a substituted or unsubstituted naphthyl, a substituted or unsubstituted biphenyl, a substituted or unsubstituted terphenyl, a substituted or unsubstituted carbazolyl, a substituted or unsubstituted dibenzothiophenyl, a substituted or unsubstituted benzothiophenyl, a substituted or unsubstituted dibenzofuranyl, a substituted or unsubstituted benzofuranyl, a substituted or unsubstituted naphthyridinyl, a substituted or unsubstituted fluorenyl, a substituted or unsubstituted benzofluorenyl, a substituted or unsubstituted triphenylenyl, a substituted or unsubstituted benzonaphthofuranyl, or a substituted or unsubstituted benzonaphthothiophenyl. For example, Ar may represent phenyl, naphthyl, biphenyl, terphenyl, dimethylfluorenyl, dimethylbenzofluorenyl, triphenylenyl, dibenzofuranyl, dibenzothiophenyl, carbazolyl unsubstituted or substituted with a phenyl(s), or benzonaphthofuranyl.

In formula 1, L₁ represents a single bond, a substituted or unsubstituted (C6-C30)arylene, or a substituted or unsubstituted (3- to 30-membered)heteroarylene. According to one embodiment of the present disclosure, L₁ represents a single bond, a substituted or unsubstituted (C6-C25)arylene, or a substituted or unsubstituted (5- to 25-membered)heteroarylene. According to another embodiment of the present disclosure, L₁ represents a single bond, an unsubstituted (C6-C18)arylene, or an unsubstituted (5- to 20-membered)heteroarylene. Specifically, L₁ may represent a single bond, phenylene, naphthylene, biphenylene, or phenanthroxazolylene.

In formula 1, X₁ to X₈, each independently, represent hydrogen, deuterium, a halogen, a cyano, a carboxyl, a nitro, a hydroxyl, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, a substituted or unsubstituted (3- to 30-membered)heteroaryl, —NR₅R₆, or —SiR₇R₈R₉; or adjacent ones of X₁ to X₈ may be linked to each other to form a ring(s); with the proviso that at least one pair of X₁ and X₂, X₂ and X₃, X₃ and X₄, X₄ and X₅, X₅ and X₆, X₆ and X₇, and X₇ and X₈ may be linked to each other to form a ring(s), in which the ring has 1 to 5 monocyclic rings. According to one embodiment of the present disclosure, X₁ to X₈, each independently, represent hydrogen; or at least one pair of X₁ and X₂, X₂ and X₃, X₃ and X₄, X₄ and X₅, X₅ and X₆, X₆ and X₇, and X₇ and X₈ is linked to each other to form a ring(s), in which the ring has 1 to 5 monocyclic rings, preferably, 2 to 5 monocyclic rings. For example, X₁ and X₂ are linked to each other to form an indole ring, in which the ring has 2 monocyclic rings. The ring may be a substituted or unsubstituted mono- or polycyclic (3- to 30-membered) alicyclic or aromatic ring, or a combination thereof; preferably a substituted or unsubstituted mono- or polycyclic (3- to 20-membered) alicyclic or aromatic ring, or the combination thereof; and more preferably a substituted or unsubstituted monocyclic (3- to 8-membered) aromatic ring. Specifically, the ring may be one in which 1 to 5 monocyclic rings, preferably 2 to 5 monocyclic rings, are fused. In addition, the ring may contain at least one heteroatom selected from B, N, O, S, Si and P; preferably, at least one heteroatom selected from N, O, and S; and more preferably, at least one heteroatom selected from N and S. When X₁ to X₈ may be linked to adjacent substituents to form a ring, the compound represented by formula 1 may be a fused carbazole-based compound, a fused azulene-based compound, etc. For example, X₁ to X₈, each independently, represent hydrogen; or may be linked to an adjacent substituent(s) to form a benzene ring, an indole ring substituted with a phenyl(s) and/or a biphenyl(s), a benzothiophene ring, a benzoindole ring substituted with at least one of a phenyl(s) and a naphthyl(s), a 15-membered polycyclic ring, a nitrogen-containing 18-membered polycyclic ring, or a nitrogen-containing 22-membered polycyclic ring.

R₅ to R₉, each independently, represent hydrogen, deuterium, a halogen, a cyano, a carboxyl, a nitro, a hydroxyl, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, or a substituted or unsubstituted (3- to 30-membered)heteroaryl; or adjacent ones of R₅ to R₉ may be linked to each other to form a ring(s).

According to one embodiment of the present disclosure, the formula 1 may be represented by any one of the following formulas 1-1 to 1-10.

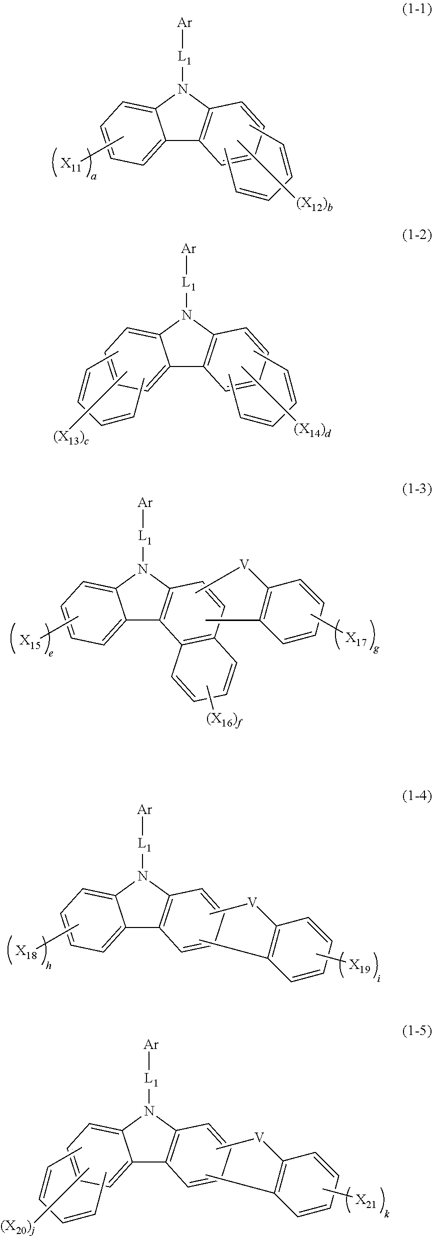

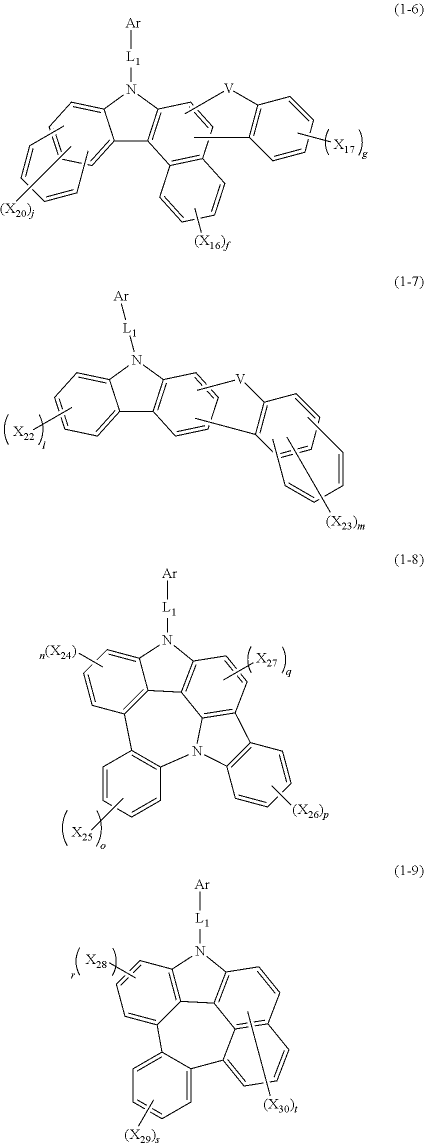

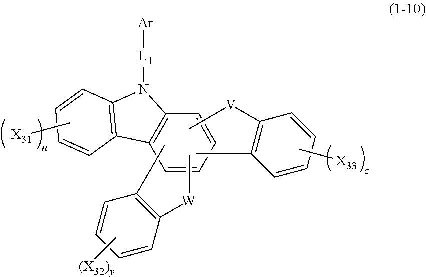

In formulas 1-1 to 1-10, the definition of the substituent is as follows.

Ar and L₁ are as defined in formula 1.

V and W, each independently, represent CR₁₂R₁₃, NR₁₄, O, or S. According to one embodiment of the present disclosure, V and W, each independently, represent NR₁₄, O, or S. For example, V may represent NR₁₄ or S, and W may represent S.

R₁₂ to R₁₄, X₁₁ to X₂₃, and X₃₁ to X₃₃, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino.

According to one embodiment of the present disclosure, R₁₂ to R₁₄, each independently, represent a substituted or unsubstituted (C1-C20)alkyl, a substituted or unsubstituted (C6-C25)aryl, or a substituted or unsubstituted (5- to 25-membered)heteroaryl. According to another embodiment of the present disclosure, R₁₂ to R₁₄, each independently, represent an unsubstituted (C6-C18)aryl. For example, R₁₂ to R₁₄, each independently, may represent phenyl or biphenyl.

According to one embodiment of the present disclosure, X₁₁ to X₂₃, and X₃₁ to X₃₃, each independently, represent hydrogen, deuterium, a substituted or unsubstituted (C1-C20)alkyl, a substituted or unsubstituted (C6-C25)aryl, or a substituted or unsubstituted (5- to 25-membered)heteroaryl. According to another embodiment of the present disclosure, X₁₁ to X₂₃, and X₃₁ to X₃₃, each independently, represent hydrogen, deuterium, or an unsubstituted (C6-C18)aryl. For example, X₁₁ to X₂₃, and X₃₁ to X₃₃, each independently, may represent hydrogen or phenyl.

X₂₄ to X₃₀, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or adjacent ones of X₂₄ to X₃₀ may be linked to each other to form a ring(s). According to one embodiment of the present disclosure, X₂₄ to X₃₀, each independently, represent hydrogen, deuterium, a substituted or unsubstituted (C1-C20)alkyl, a substituted or unsubstituted (C6-C25)aryl, or a substituted or unsubstituted (5- to 25-membered)heteroaryl; or adjacent ones of X₂₄ to X₃₀ may be linked to each other to form a ring(s). According to another embodiment of the present disclosure, X₂₄ to X₃₀, each independently, represent hydrogen, deuterium, or an unsubstituted (C6-C18)aryl; or adjacent ones of X₂₄ to X₃₀ may be linked to each other to form a ring(s). For example, X₂₄ to X₃₀, each independently, represent hydrogen; or adjacent ones of X₂₄ to X₃₀ may be linked to each other to form a benzene ring.

a, e to i, k, l, o, p, s, u, y, and z, each independently, represent an integer of 1 to 4; b to d, j, and m, each independently, represent an integer of 1 to 6; n and r, each independently, represent an integer of 1 to 3; q represents an integer of 1 or 2; t represents an integer of 1 to 5; where a to u, y, and z, each independently, are an integer of 2 or more, each of X₁₁ to X₃₃ may be the same or different.

In formula 2, X represents —N═, —NR₁₀—, —O—, or —S—; Y represents —N═, —NR₁₁—, —O—, or —S—; with the proviso that when X represents —N═, Y represents —NR₁₁—, —O—, or —S—, and when X represents —NR₁₀—, Y represents —N═, —O—, or —S—. According to one embodiment of the present disclosure, X represents —N═, —NR₁₀—, —O—, or —S—; Y represents —N═, —NR₁₁—, —O—, or —S—; with the proviso that any one of X and Y represents —N═.

R₁₀ and R₁₁, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino. According to one embodiment of the present disclosure, R₁₀ and R₁₁, each independently, represent a substituted or unsubstituted (C1-C20)alkyl, a substituted or unsubstituted (C6-C25)aryl, or a substituted or unsubstituted (5- to 25-membered)heteroaryl. According to another embodiment of the present disclosure, R₁₀ and R₁₁, each independently, represent an unsubstituted (C6-C18)aryl. For example, R₁₀ and R₁₁ may be a phenyl.

In formula 2, HAr represents a substituted or unsubstituted (3- to 30-membered)heteroaryl containing a nitrogen atom(s). According to one embodiment of the present disclosure, HAr represents a substituted or unsubstituted (5- to 25-membered)heteroaryl containing a nitrogen atom(s). According to another embodiment of the present disclosure, HAr represents a (5- to 20-membered)heteroaryl containing a nitrogen atom(s), unsubstituted or substituted with a (5- to 25-membered)heteroaryl(s) and/or a (C6-C25)aryl(s). Specifically, HAr represents a substituted or unsubstituted triazinyl, a substituted or unsubstituted pyridyl, a substituted or unsubstituted pyrimidinyl, a substituted or unsubstituted quinazolinyl, a substituted or unsubstituted benzoquinazolinyl, a substituted or unsubstituted quinoxalinyl, a substituted or unsubstituted benzoquinoxalinyl, a substituted or unsubstituted quinolyl, a substituted or unsubstituted benzoquinolyl, a substituted or unsubstituted isoquinolyl, a substituted or unsubstituted benzoisoquinolyl, a substituted or unsubstituted triazolyl, a substituted or unsubstituted pyrazolyl, a substituted or unsubstituted naphthyridinyl, or a substituted or unsubstituted benzothienopyrimidinyl. For example, HAr may represent a substituted triazinyl, a substituted pyrimidinyl, a substituted quinoxalinyl, a substituted quinazolinyl, or a substituted naphthyridinyl. The substituent of the substituted triazinyl, the substituted pyrimidinyl, the substituted quinoxalinyl, the substituted quinazolinyl, and the substituted naphthyridinyl may be at least one of phenyl unsubstituted or substituted with a diphenylamino(s), naphthyl, biphenyl, dimethylfluorenyl, dimethylbenzofluorenyl, dibenzothiophenyl, dibenzofuranyl, benzonaphthothiophenyl, phenylcarbazolyl, and phenylbenzocarbazolyl.

In formula 2, L₂ represents a single bond, a substituted or unsubstituted (C6-C30)arylene, or a substituted or unsubstituted (3- to 30-membered)heteroarylene. According to one embodiment of the present disclosure, L₂ represents a single bond, a substituted or unsubstituted (C6-C25)arylene, or a substituted or unsubstituted (5- to 25-membered)heteroarylene. According to another embodiment of the present disclosure, L₂ represents a single bond, an unsubstituted (C6-C18)arylene, or an unsubstituted (5- to 20-membered)heteroarylene. For example, L₂ may represent a single bond, phenylene, or pyridylene.

In formula 2, R₁ represents a substituted or unsubstituted (C6-C30)aryl, or a substituted or unsubstituted (3- to 30-membered)heteroaryl. According to one embodiment of the present disclosure, R₁ represents a substituted or unsubstituted (C6-C30)aryl, or a substituted or unsubstituted (5- to 25-membered)heteroaryl. According to another embodiment of the present disclosure, R₁ represents a (C6-C29)aryl unsubstituted or substituted with a (C1-C10)alkyl(s) and/or a (C6-C18)aryl(s); or a (5- to 25-membered)heteroaryl unsubstituted or substituted with a (C6-C18)aryl(s). For example, R₁ may be phenyl, naphthyl, phenylnaphthyl, biphenyl, dimethylfluorenyl, dimethylbenzofluorenyl, spirobifluorenyl, spiro[fluorene-benzofluorene]yl, phenylcarbazolyl, phenylbenzocarbazolyl, dibenzofuranyl, or dibenzothiophenyl.

In formula 2, R₂ to R₄, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or adjacent ones of R₂ to R₄ may be linked to each other to form a ring(s). For example, R₂ to R₄ may be hydrogen.

In formula 2, a′ represents an integer of 1; b′ and c′, each independently, represent an integer of 1 or 2; d′ represents an integer of 1 to 4; where if b′, c′, and d′, each independently, are an integer of 2 or more, each of R₂ to R₄ may be the same or different.

According to one embodiment of the present disclosure, the formula 2 may be represented by any one of the following formulas 2-1 and 2-2.

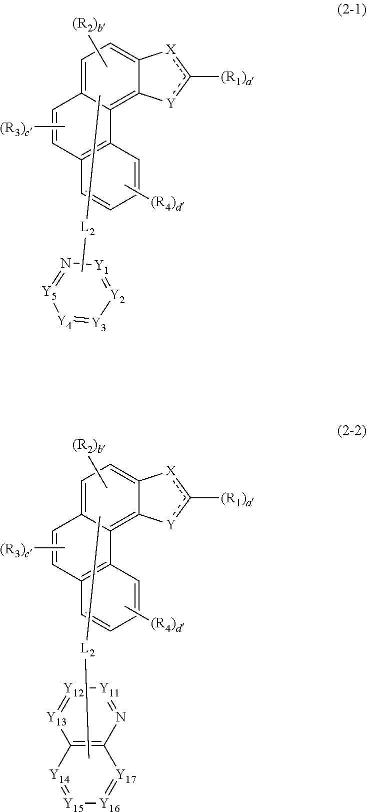

In formulas 2-1 and 2-2, X, Y, R₁ to R₄, L₂, and a′ to d′ are as defined in formula 2.

In formulas 2-1 and 2-2, Y₁ to Y₅, and Y₁₁ to Y₁₇, each independently, represent N or CR₁₅. According to one embodiment of the present disclosure, at least one of Y₁ to Y₅ represents CR₁₅, and at least one of Y₁₁ to Y₁₇ represents CR₁₅.

R₁₅, each independently, represents hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or adjacent ones of R₁₅'s may be linked to each other to form a ring(s). According to one embodiment of the present disclosure, R₁₅, each independently, represents hydrogen, deuterium, a substituted or unsubstituted (C1-C30)alkyl, a substituted or unsubstituted (C6-C25)aryl, or a substituted or unsubstituted (5- to 25-membered)heteroaryl. According to another embodiment of the present disclosure, R₁₅, each independently, represents hydrogen; deuterium; a (C6-C18)aryl unsubstituted or substituted with a (C1-C10)alkyl(s) and/or a di(C6-C18)arylamino(s); or a (5- to 20-membered)heteroaryl unsubstituted or substituted with a (C6-C18)aryl(s). For example, R₁₅, each independently, may represent hydrogen, phenyl unsubstituted or substituted with a diphenylamino(s), naphthyl, biphenyl, dimethylfluorenyl, dimethylbenzofluorenyl, dibenzothiophenyl, dibenzofuranyl, benzonaphthothiophenyl, phenylcarbazolyl, or phenylbenzocarbazolyl.

The compound represented by formula 1 includes the following compounds, but is not limited thereto.

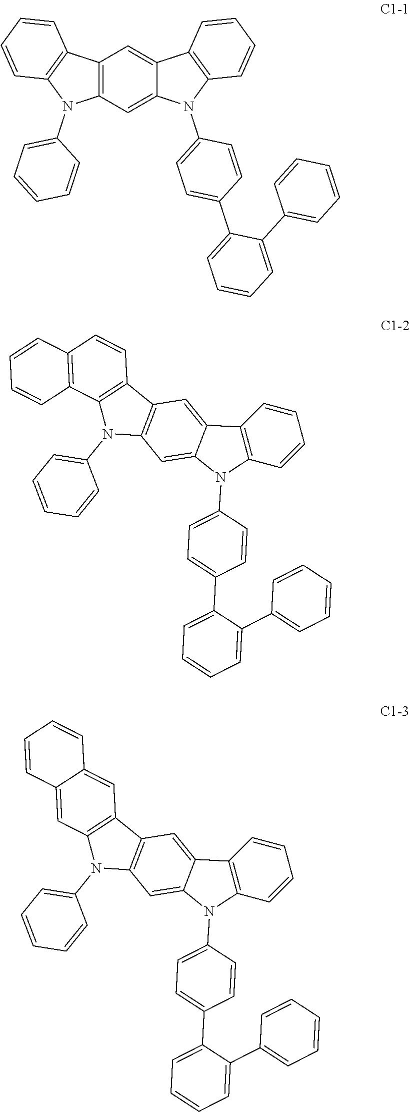 [SMILES: c1ccc(-c2ccccc2-c2ccc(-n3c4ccccc4c4cc5c6ccccc6n(-c6ccccc6)c5cc43)cc2)cc1 ; c1ccc(-c2ccccc2-c2ccc(-n3c4ccccc4c4cc5c6ccc7ccccc7c6n(-c6ccccc6)c5cc43)cc2)cc1 ; c1ccc(-c2ccccc2-c2ccc(-n3c4ccccc4c4cc5c6cc7ccccc7cc6n(-c6ccccc6)c5cc43)cc2)cc1]

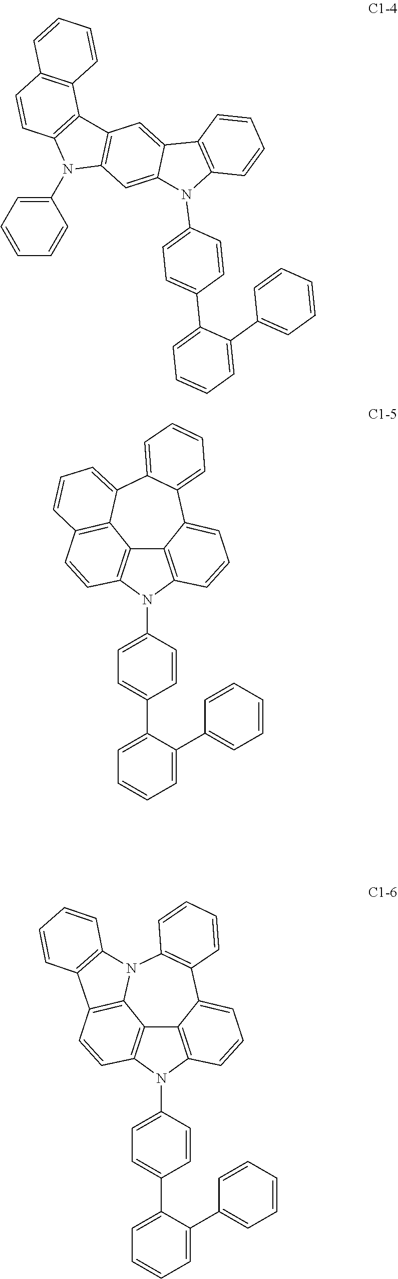

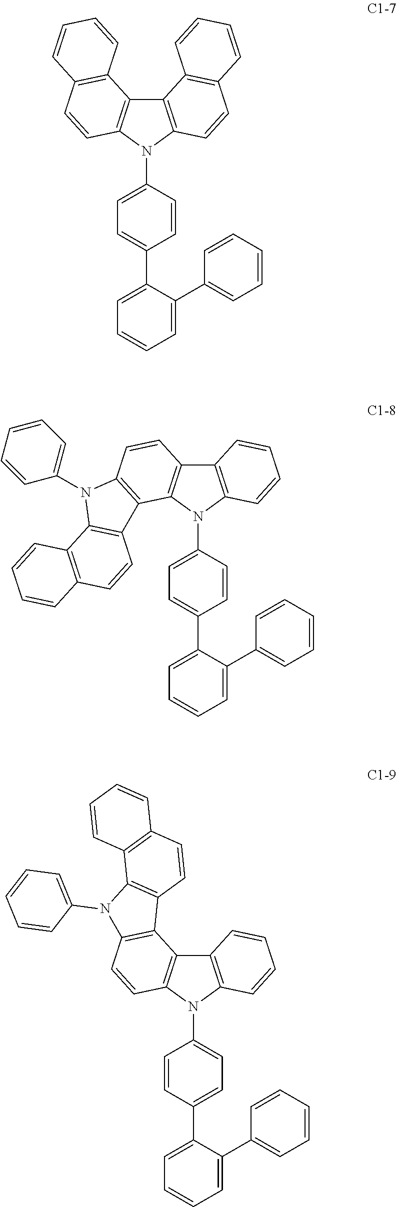 [SMILES: c1ccc(-c2ccccc2-c2ccc(-n3c4ccc5ccccc5c4c4c5ccccc5ccc43)cc2)cc1 ; c1ccc(-c2ccccc2-c2ccc(-n3c4ccccc4c4ccc5c(c6ccc7ccccc7c6n5-c5ccccc5)c43)cc2)cc1 ; c1ccc(-c2ccccc2-c2ccc(-n3c4ccccc4c4c5c6ccc7ccccc7c6n(-c6ccccc6)c5ccc43)cc2)cc1]

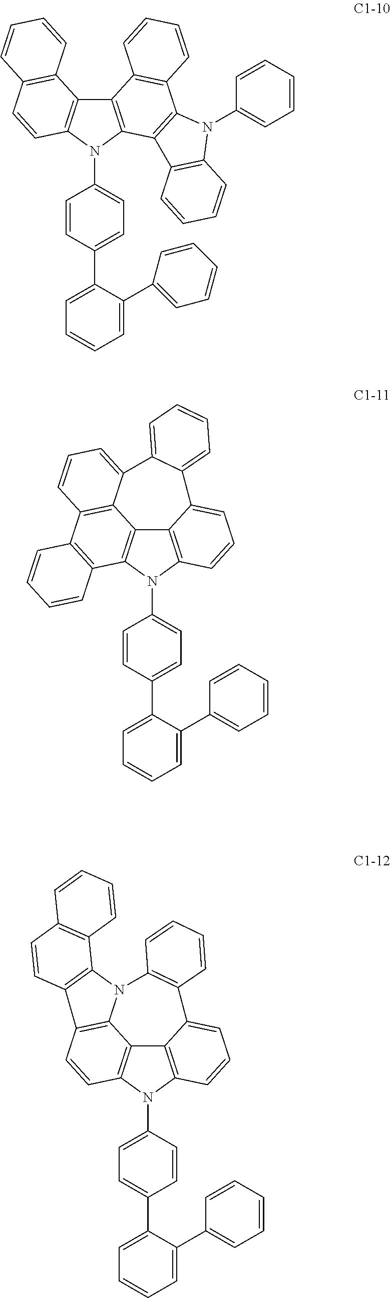 [SMILES: c1ccc(-c2ccccc2-c2ccc(-n3c4ccc5ccccc5c4c4c5ccccc5c5c(c6ccccc6n5-c5ccccc5)c43)cc2)cc1 ; c1ccc(-c2ccccc2-c2ccc(-n3c4cccc5c4c4c6c(cccc6c6ccccc6c43)-c3ccccc3-5)cc2)cc1 ; c1ccc(-c2ccccc2-c2ccc(-n3c4cccc5c6ccccc6n6c7c8ccccc8ccc7c7ccc3c(c54)c76)cc2)cc1]

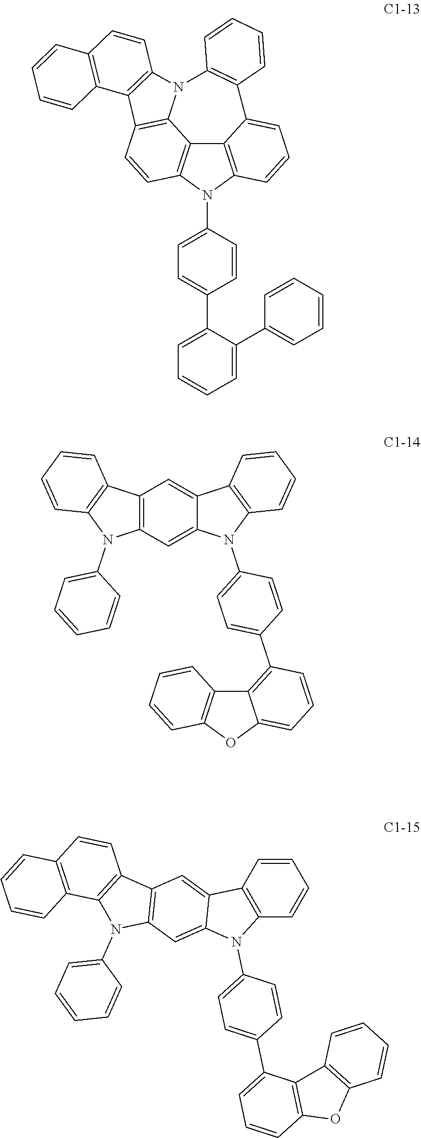 [SMILES: c1ccc(-c2ccccc2-c2ccc(-n3c4cccc5c6ccccc6n6c7ccc8ccccc8c7c7ccc3c(c54)c76)cc2)cc1 ; c1ccc(-n2c3ccccc3c3cc4c5ccccc5n(-c5ccc(-c6cccc7oc8ccccc8c67)cc5)c4cc32)cc1 ; c1ccc(-n2c3cc4c(cc3c3ccc5ccccc5c32)c2ccccc2n4-c2ccc(-c3cccc4oc5ccccc5c34)cc2)cc1]

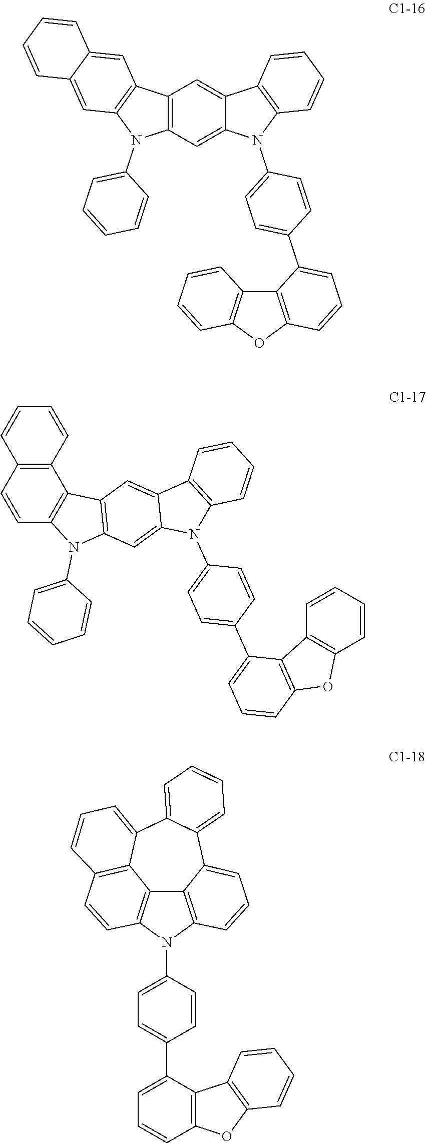 [SMILES: c1ccc(-n2c3cc4ccccc4cc3c3cc4c5ccccc5n(-c5ccc(-c6cccc7oc8ccccc8c67)cc5)c4cc32)cc1 ; c1ccc(-n2c3cc4c(cc3c3c5ccccc5ccc32)c2ccccc2n4-c2ccc(-c3cccc4oc5ccccc5c34)cc2)cc1 ; c1ccc2c(c1)-c1cccc3ccc4c(c13)c1c-2cccc1n4-c1ccc(-c2cccc3oc4ccccc4c23)cc1]

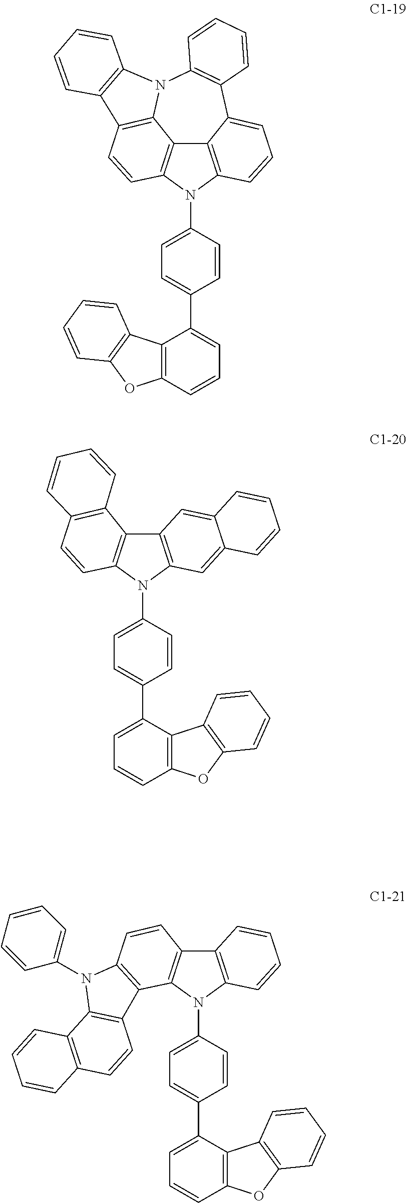 [SMILES: c1ccc2c(c1)oc1cccc(-c3ccc(-n4c5cccc6c7ccccc7n7c8ccccc8c8ccc4c(c65)c87)cc3)c12 ; c1ccc2cc3c(cc2c1)c1c2ccccc2ccc1n3-c1ccc(-c2cccc3oc4ccccc4c23)cc1 ; c1ccc(-n2c3ccc4c5ccccc5n(-c5ccc(-c6cccc7oc8ccccc8c67)cc5)c4c3c3ccc4ccccc4c32)cc1]

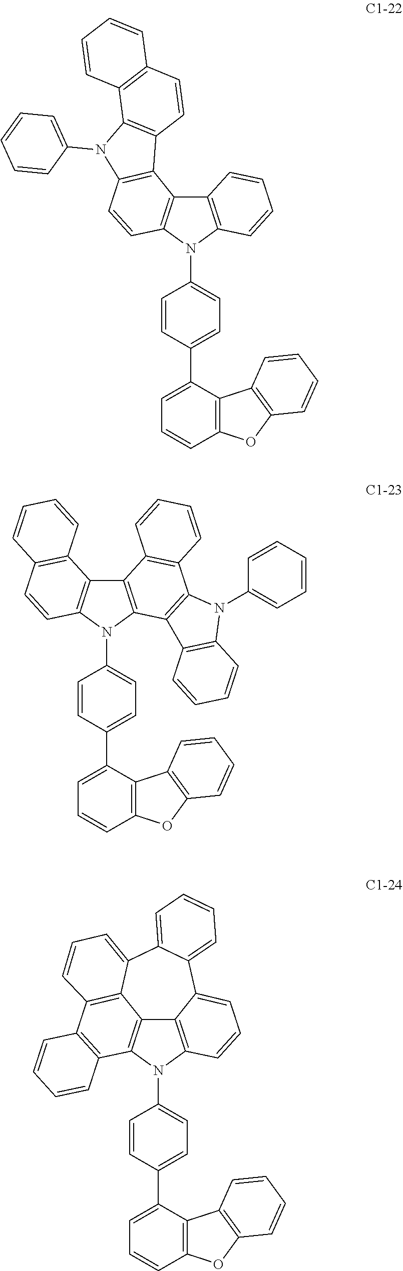 [SMILES: c1ccc(-n2c3ccc4c(c5ccccc5n4-c4ccc(-c5cccc6oc7ccccc7c56)cc4)c3c3ccc4ccccc4c32)cc1 ; c1ccc(-n2c3ccccc3c3c2c2ccccc2c2c4c5ccccc5ccc4n(-c4ccc(-c5cccc6oc7ccccc7c56)cc4)c23)cc1 ; c1ccc2c(c1)-c1cccc3c4ccccc4c4c(c13)c1c-2cccc1n4-c1ccc(-c2cccc3oc4ccccc4c23)cc1]

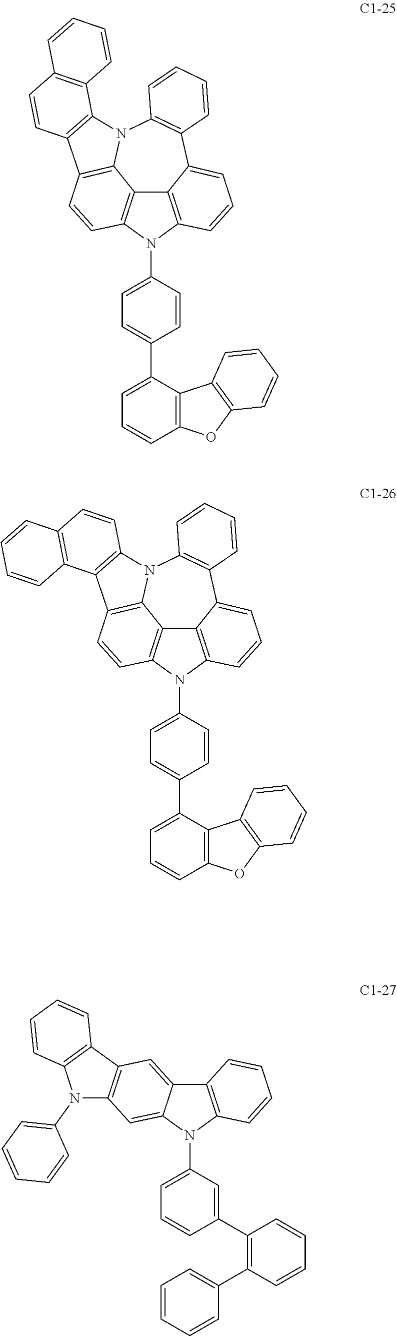 [SMILES: c1ccc2c(c1)ccc1c3ccc4c5c6c(cccc6n4-c4ccc(-c6cccc7oc8ccccc8c67)cc4)c4ccccc4n(c21)c35 ; c1ccc2c(c1)ccc1c2c2ccc3c4c5c(cccc5n3-c3ccc(-c5cccc6oc7ccccc7c56)cc3)c3ccccc3n1c24 ; c1ccc(-c2ccccc2-c2cccc(-n3c4ccccc4c4cc5c6ccccc6n(-c6ccccc6)c5cc43)c2)cc1]

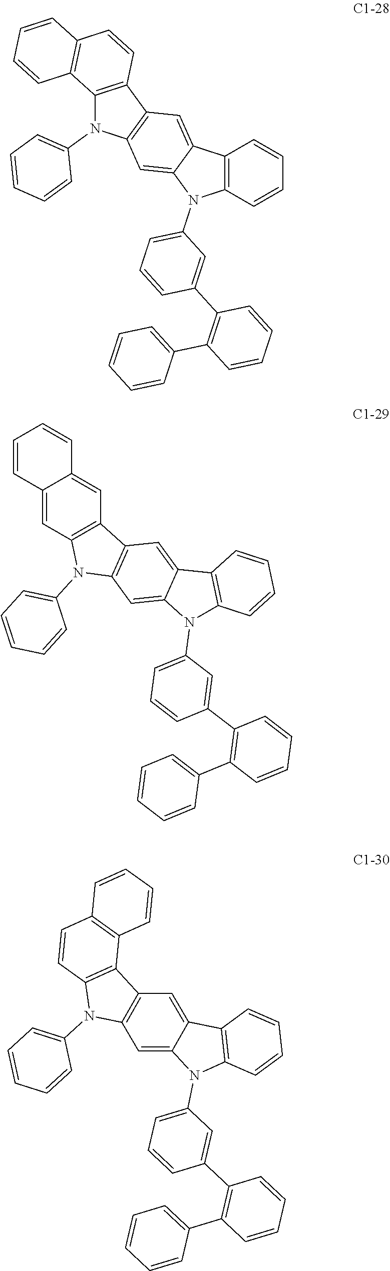 [SMILES: c1ccc(-c2ccccc2-c2cccc(-n3c4ccccc4c4cc5c6ccc7ccccc7c6n(-c6ccccc6)c5cc43)c2)cc1 ; c1ccc(-c2ccccc2-c2cccc(-n3c4ccccc4c4cc5c6cc7ccccc7cc6n(-c6ccccc6)c5cc43)c2)cc1 ; c1ccc(-c2ccccc2-c2cccc(-n3c4ccccc4c4cc5c6c7ccccc7ccc6n(-c6ccccc6)c5cc43)c2)cc1]

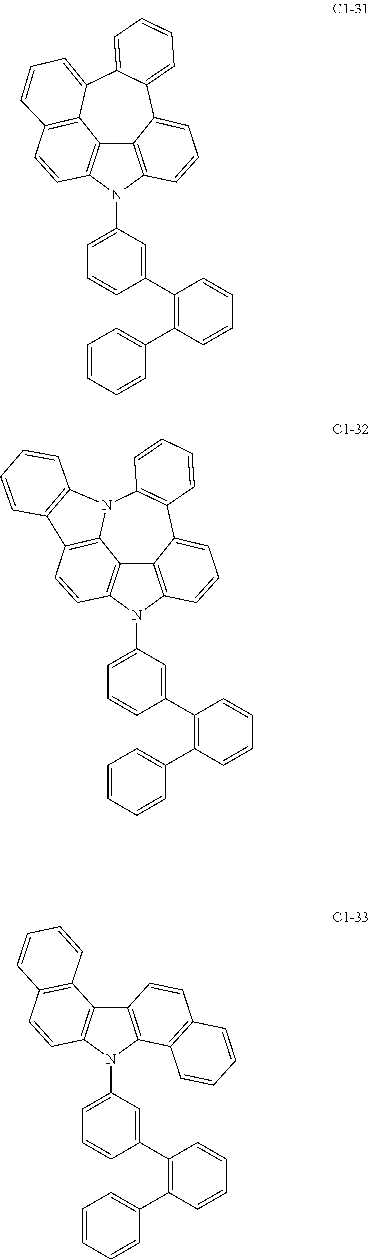 [SMILES: c1ccc(-c2ccccc2-c2cccc(-n3c4cccc5c4c4c6c(cccc6ccc43)-c3ccccc3-5)c2)cc1 ; c1ccc(-c2ccccc2-c2cccc(-n3c4cccc5c6ccccc6n6c7ccccc7c7ccc3c(c54)c76)c2)cc1 ; c1ccc(-c2ccccc2-c2cccc(-n3c4ccc5ccccc5c4c4ccc5ccccc5c43)c2)cc1]

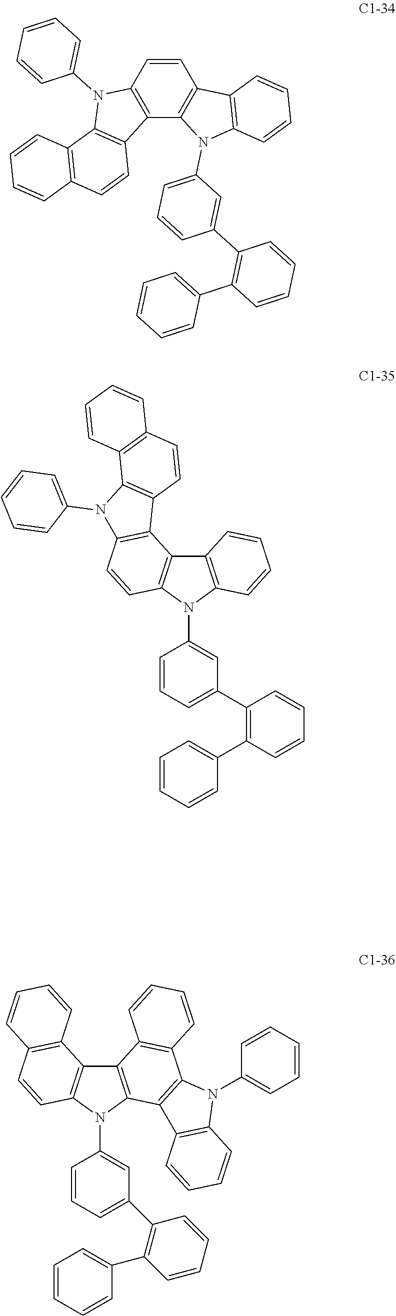 [SMILES: c1ccc(-c2ccccc2-c2cccc(-n3c4ccccc4c4ccc5c(c6ccc7ccccc7c6n5-c5ccccc5)c43)c2)cc1 ; c1ccc(-c2ccccc2-c2cccc(-n3c4ccccc4c4c5c6ccc7ccccc7c6n(-c6ccccc6)c5ccc43)c2)cc1 ; c1ccc(-c2ccccc2-c2cccc(-n3c4ccc5ccccc5c4c4c5ccccc5c5c(c6ccccc6n5-c5ccccc5)c43)c2)cc1]

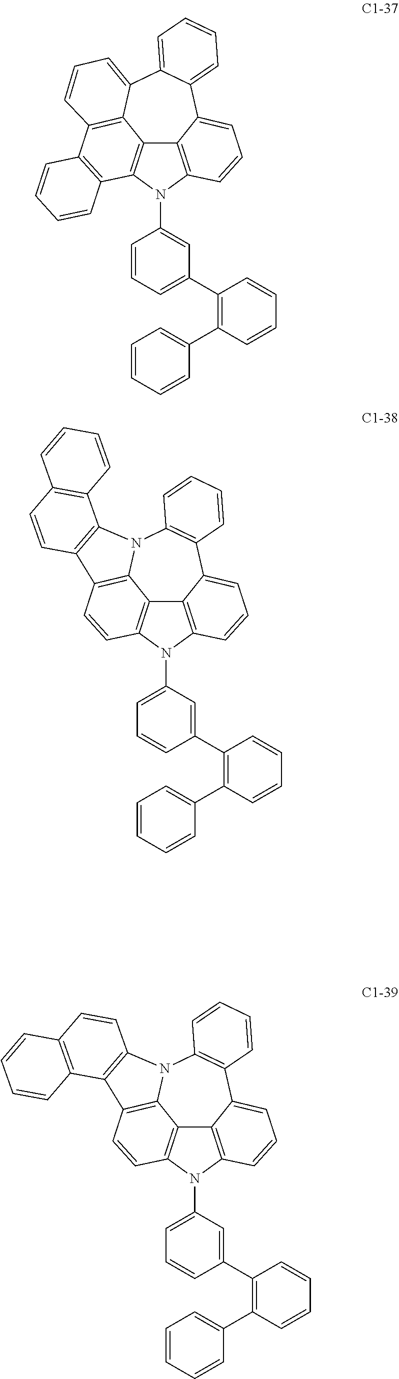 [SMILES: c1ccc(-c2ccccc2-c2cccc(-n3c4cccc5c4c4c6c(cccc6c6ccccc6c43)-c3ccccc3-5)c2)cc1 ; c1ccc(-c2ccccc2-c2cccc(-n3c4cccc5c6ccccc6n6c7c8ccccc8ccc7c7ccc3c(c54)c76)c2)cc1 ; c1ccc(-c2ccccc2-c2cccc(-n3c4cccc5c6ccccc6n6c7ccc8ccccc8c7c7ccc3c(c54)c76)c2)cc1]

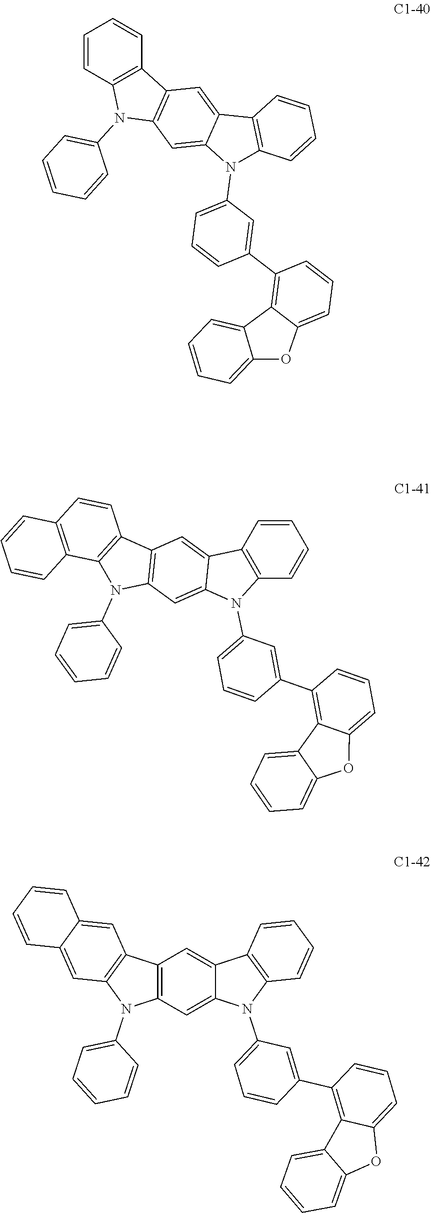 [SMILES: c1ccc(-n2c3ccccc3c3cc4c5ccccc5n(-c5cccc(-c6cccc7oc8ccccc8c67)c5)c4cc32)cc1 ; c1ccc(-n2c3cc4c(cc3c3ccc5ccccc5c32)c2ccccc2n4-c2cccc(-c3cccc4oc5ccccc5c34)c2)cc1 ; c1ccc(-n2c3cc4ccccc4cc3c3cc4c5ccccc5n(-c5cccc(-c6cccc7oc8ccccc8c67)c5)c4cc32)cc1]

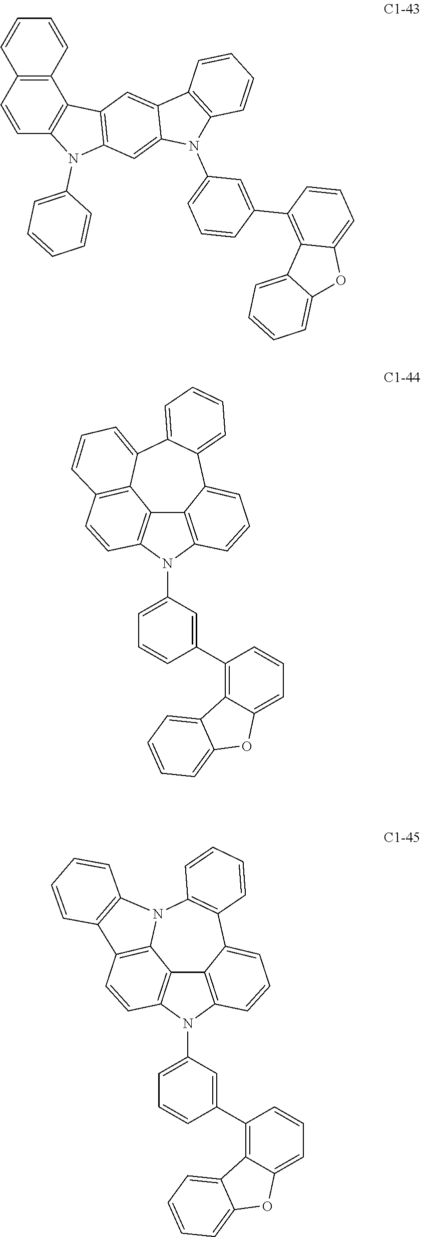 [SMILES: c1ccc(-n2c3cc4c(cc3c3c5ccccc5ccc32)c2ccccc2n4-c2cccc(-c3cccc4oc5ccccc5c34)c2)cc1 ; c1cc(-c2cccc3oc4ccccc4c23)cc(-n2c3cccc4c3c3c5c(cccc5ccc32)-c2ccccc2-4)c1 ; c1cc(-c2cccc3oc4ccccc4c23)cc(-n2c3cccc4c5ccccc5n5c6ccccc6c6ccc2c(c43)c65)c1]

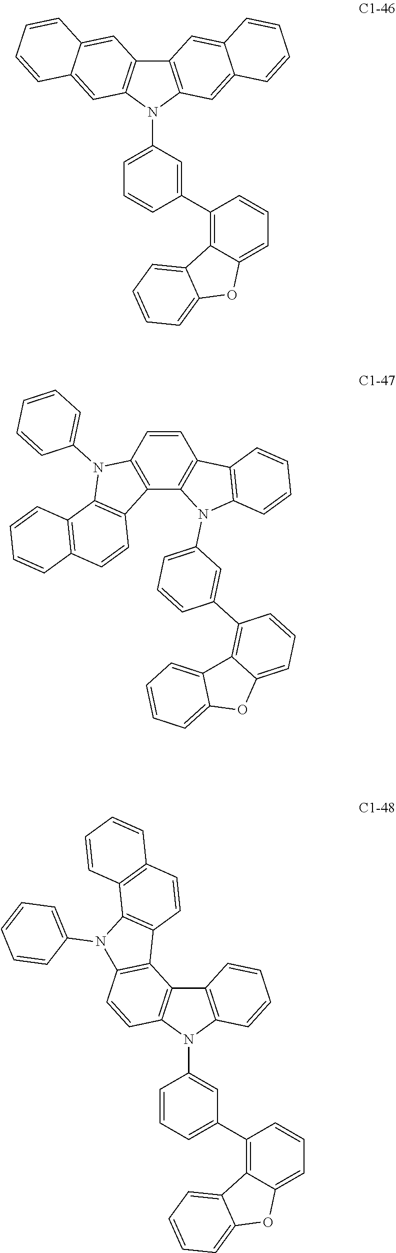 [SMILES: c1cc(-c2cccc3oc4ccccc4c23)cc(-n2c3cc4ccccc4cc3c3cc4ccccc4cc32)c1 ; c1ccc(-n2c3ccc4c5ccccc5n(-c5cccc(-c6cccc7oc8ccccc8c67)c5)c4c3c3ccc4ccccc4c32)cc1 ; c1ccc(-n2c3ccc4c(c5ccccc5n4-c4cccc(-c5cccc6oc7ccccc7c56)c4)c3c3ccc4ccccc4c32)cc1]

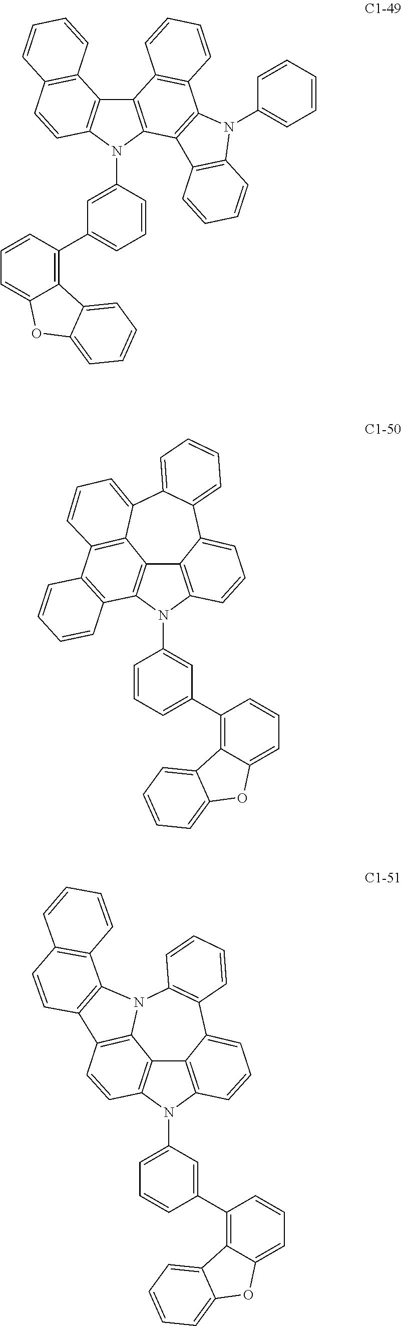 [SMILES: c1ccc(-n2c3ccccc3c3c2c2ccccc2c2c4c5ccccc5ccc4n(-c4cccc(-c5cccc6oc7ccccc7c56)c4)c23)cc1 ; c1cc(-c2cccc3oc4ccccc4c23)cc(-n2c3cccc4c3c3c5c(cccc5c5ccccc5c32)-c2ccccc2-4)c1 ; c1cc(-c2cccc3oc4ccccc4c23)cc(-n2c3cccc4c5ccccc5n5c6c7ccccc7ccc6c6ccc2c(c43)c65)c1]

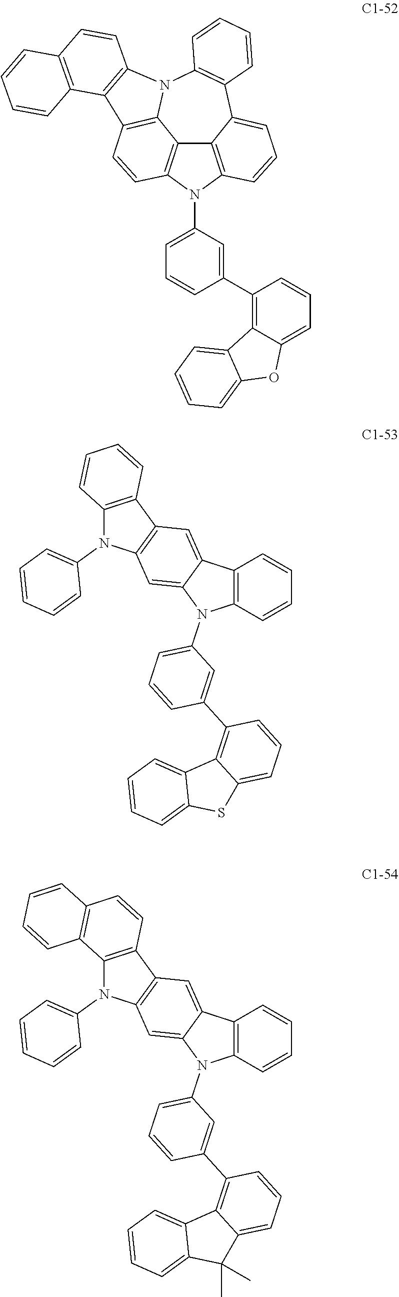 [SMILES: c1cc(-c2cccc3oc4ccccc4c23)cc(-n2c3cccc4c5ccccc5n5c6ccc7ccccc7c6c6ccc2c(c43)c65)c1 ; c1ccc(-n2c3ccccc3c3cc4c5ccccc5n(-c5cccc(-c6cccc7sc8ccccc8c67)c5)c4cc32)cc1 ; CC1(C)c2ccccc2-c2c(-c3cccc(-n4c5ccccc5c5cc6c7ccc8ccccc8c7n(-c7ccccc7)c6cc54)c3)cccc21]

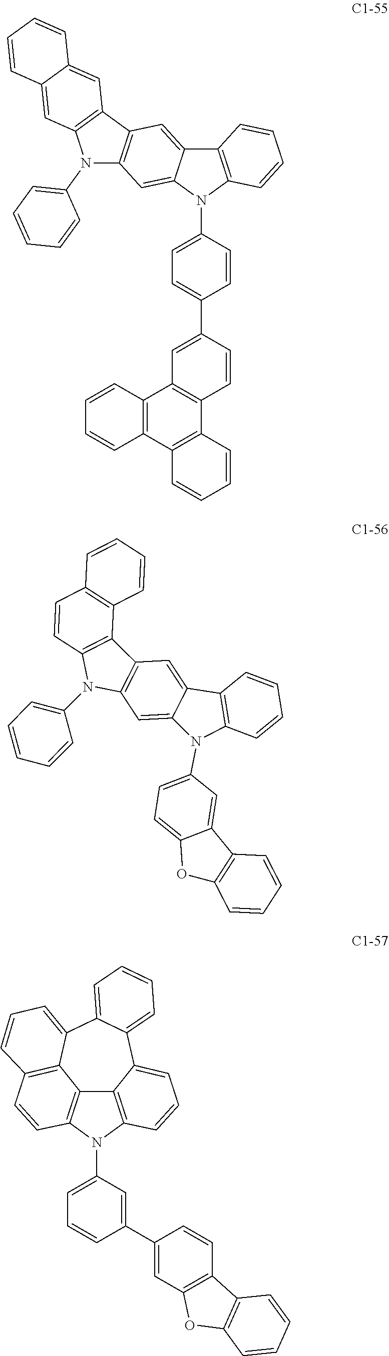 [SMILES: c1ccc(-n2c3cc4ccccc4cc3c3cc4c5ccccc5n(-c5ccc(-c6ccc7c8ccccc8c8ccccc8c7c6)cc5)c4cc32)cc1 ; c1ccc(-n2c3cc4c(cc3c3c5ccccc5ccc32)c2ccccc2n4-c2ccc3oc4ccccc4c3c2)cc1 ; c1cc(-c2ccc3c(c2)oc2ccccc23)cc(-n2c3cccc4c3c3c5c(cccc5ccc32)-c2ccccc2-4)c1]

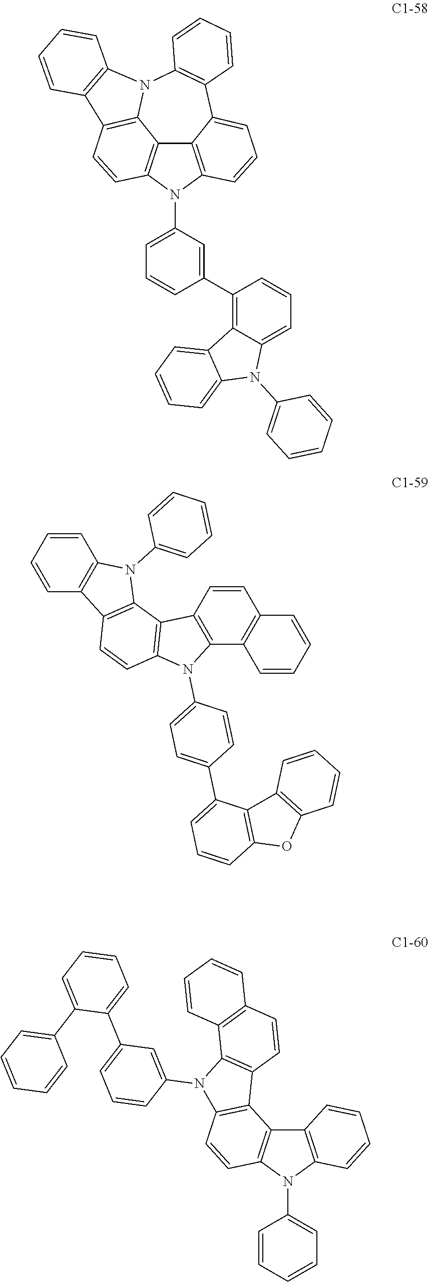 [SMILES: c1ccc(-n2c3ccccc3c3c(-c4cccc(-n5c6cccc7c8ccccc8n8c9ccccc9c9ccc5c(c76)c98)c4)cccc32)cc1 ; c1ccc(-n2c3ccccc3c3ccc4c(c5ccc6ccccc6c5n4-c4ccc(-c5cccc6oc7ccccc7c56)cc4)c32)cc1 ; c1ccc(-c2ccccc2-c2cccc(-n3c4ccc5c(c6ccccc6n5-c5ccccc5)c4c4ccc5ccccc5c43)c2)cc1]

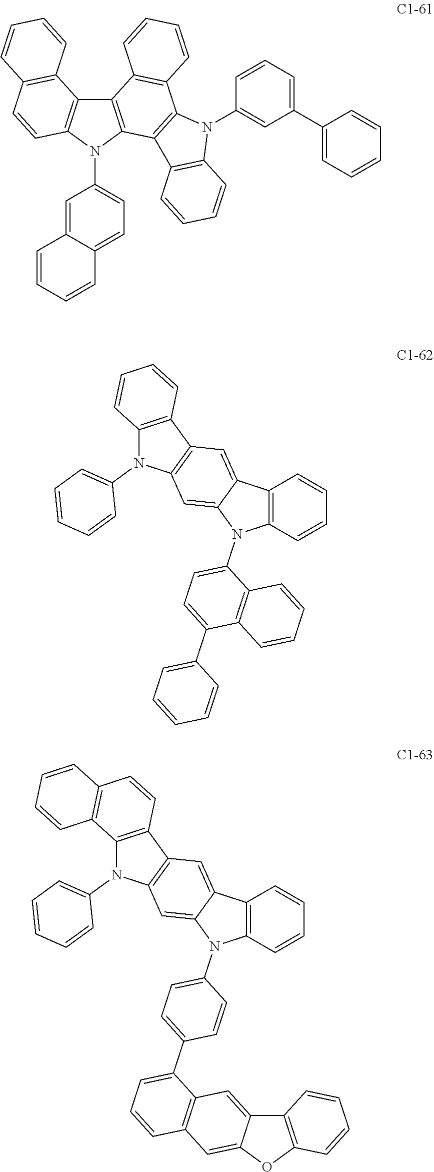 [SMILES: c1ccc(-c2cccc(-n3c4ccccc4c4c3c3ccccc3c3c5c6ccccc6ccc5n(-c5ccc6ccccc6c5)c34)c2)cc1 ; c1ccc(-c2ccc(-n3c4ccccc4c4cc5c6ccccc6n(-c6ccccc6)c5cc43)c3ccccc23)cc1 ; c1ccc(-n2c3cc4c(cc3c3ccc5ccccc5c32)c2ccccc2n4-c2ccc(-c3cccc4cc5oc6ccccc6c5cc34)cc2)cc1]

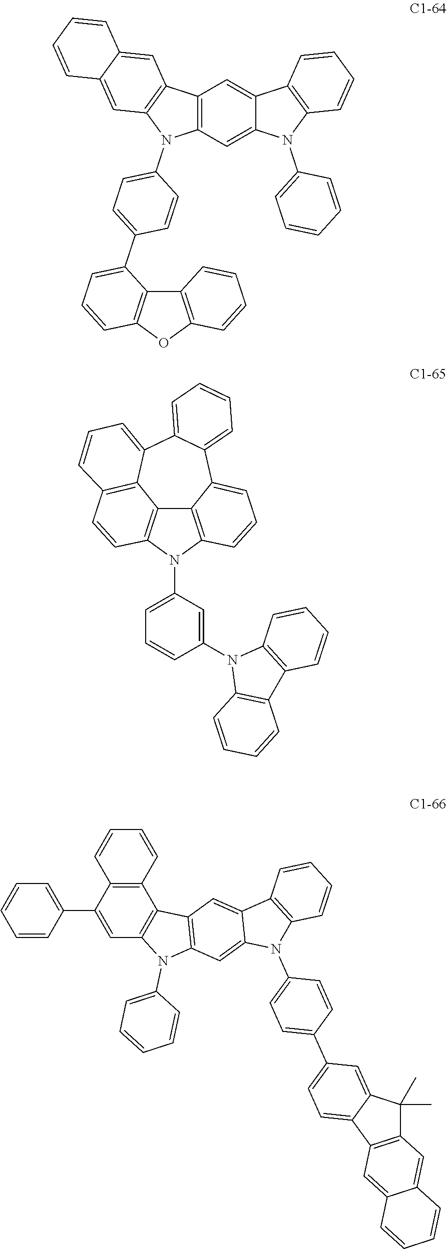 [SMILES: c1ccc(-n2c3ccccc3c3cc4c5cc6ccccc6cc5n(-c5ccc(-c6cccc7oc8ccccc8c67)cc5)c4cc32)cc1 ; c1cc(-n2c3ccccc3c3ccccc32)cc(-n2c3cccc4c3c3c5c(cccc5ccc32)-c2ccccc2-4)c1 ; CC1(C)c2cc(-c3ccc(-n4c5ccccc5c5cc6c7c8ccccc8c(-c8ccccc8)cc7n(-c7ccccc7)c6cc54)cc3)ccc2-c2cc3ccccc3cc21]

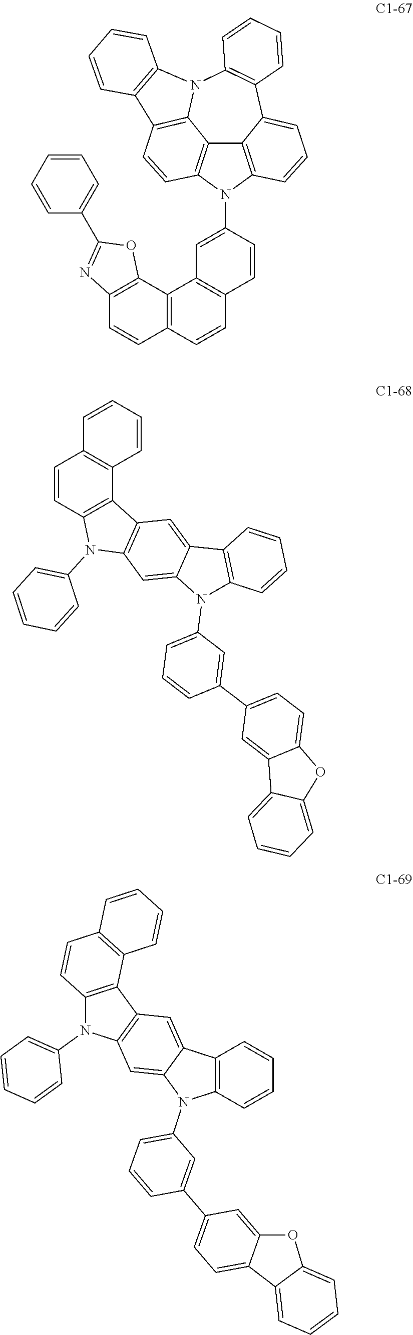 [SMILES: c1ccc(-c2nc3ccc4ccc5ccc(-n6c7cccc8c9ccccc9n9c%10ccccc%10c%10ccc6c(c87)c%109)cc5c4c3o2)cc1 ; c1ccc(-n2c3cc4c(cc3c3c5ccccc5ccc32)c2ccccc2n4-c2cccc(-c3ccc4oc5ccccc5c4c3)c2)cc1 ; c1ccc(-n2c3cc4c(cc3c3c5ccccc5ccc32)c2ccccc2n4-c2cccc(-c3ccc4c(c3)oc3ccccc34)c2)cc1]

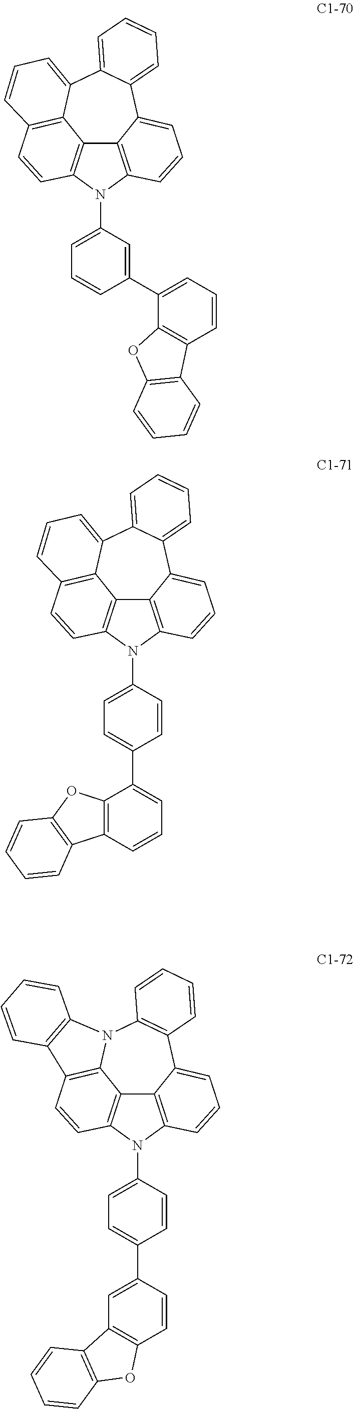

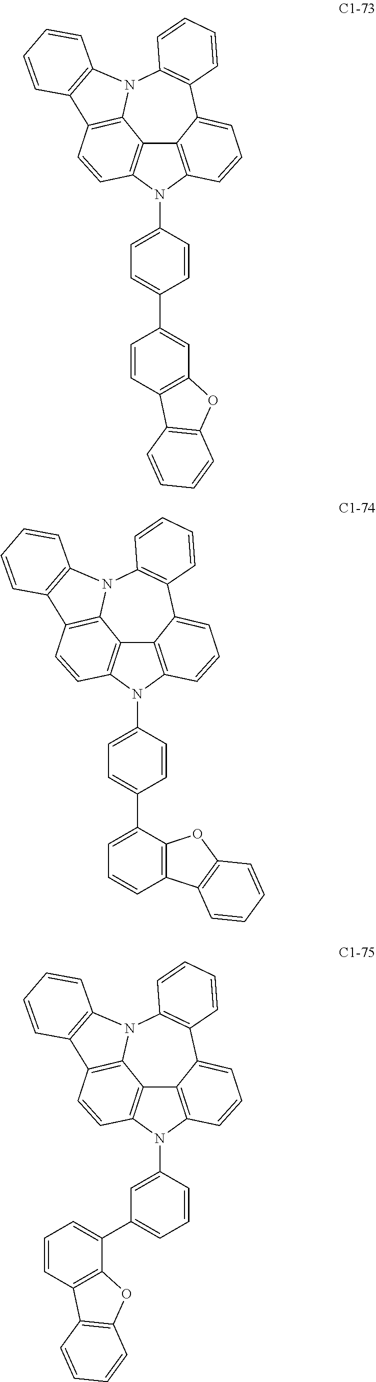

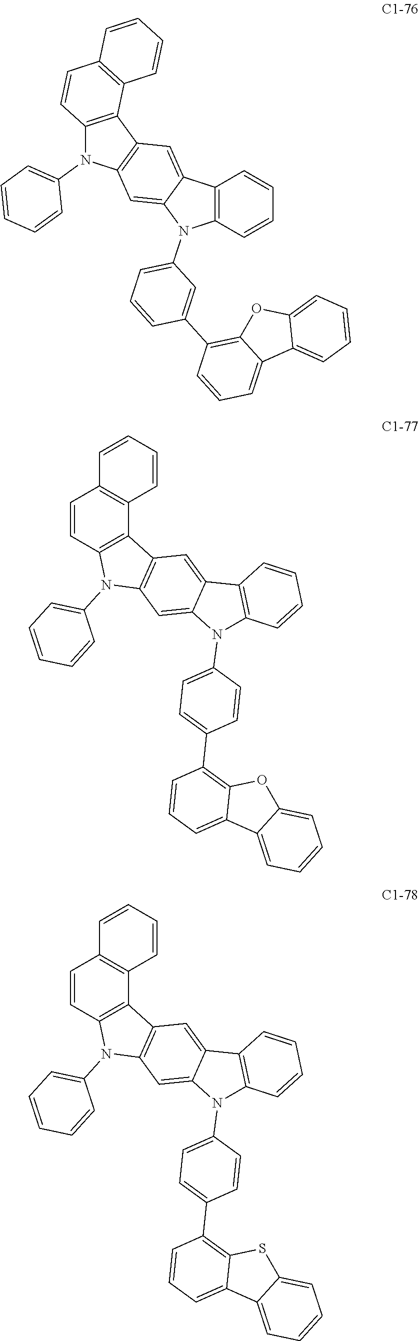 [SMILES: c1ccc(-n2c3cc4c(cc3c3c5ccccc5ccc32)c2ccccc2n4-c2cccc(-c3cccc4c3oc3ccccc34)c2)cc1 ; c1ccc(-n2c3cc4c(cc3c3c5ccccc5ccc32)c2ccccc2n4-c2ccc(-c3cccc4c3oc3ccccc34)cc2)cc1 ; c1ccc(-n2c3cc4c(cc3c3c5ccccc5ccc32)c2ccccc2n4-c2ccc(-c3cccc4c3sc3ccccc34)cc2)cc1]

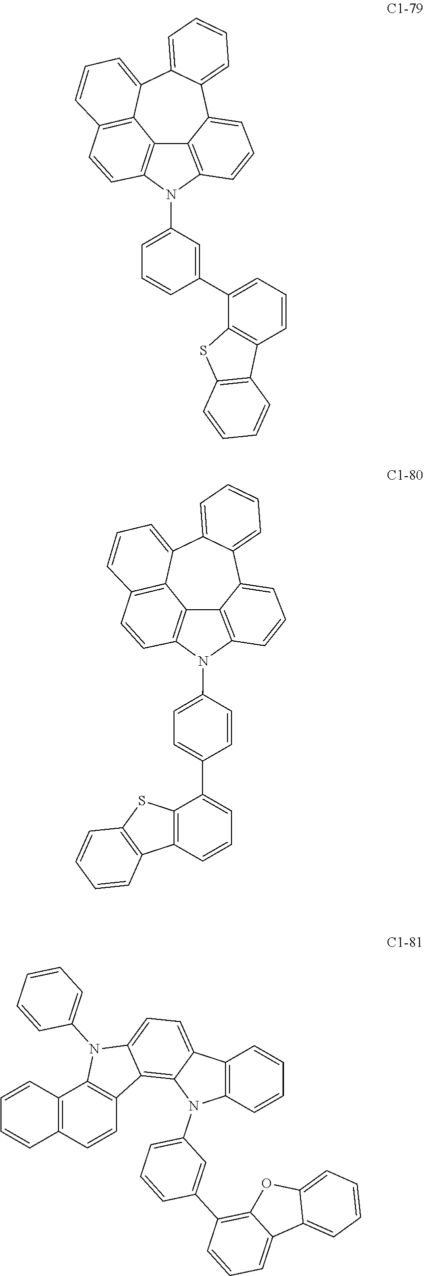

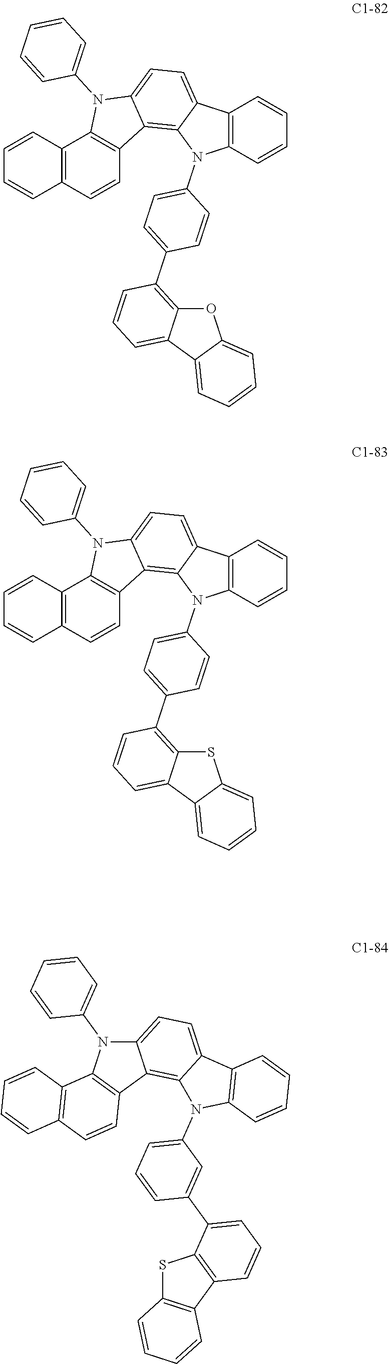 [SMILES: c1ccc(-n2c3ccc4c5ccccc5n(-c5ccc(-c6cccc7c6oc6ccccc67)cc5)c4c3c3ccc4ccccc4c32)cc1 ; c1ccc(-n2c3ccc4c5ccccc5n(-c5ccc(-c6cccc7c6sc6ccccc67)cc5)c4c3c3ccc4ccccc4c32)cc1 ; c1ccc(-n2c3ccc4c5ccccc5n(-c5cccc(-c6cccc7c6sc6ccccc67)c5)c4c3c3ccc4ccccc4c32)cc1]

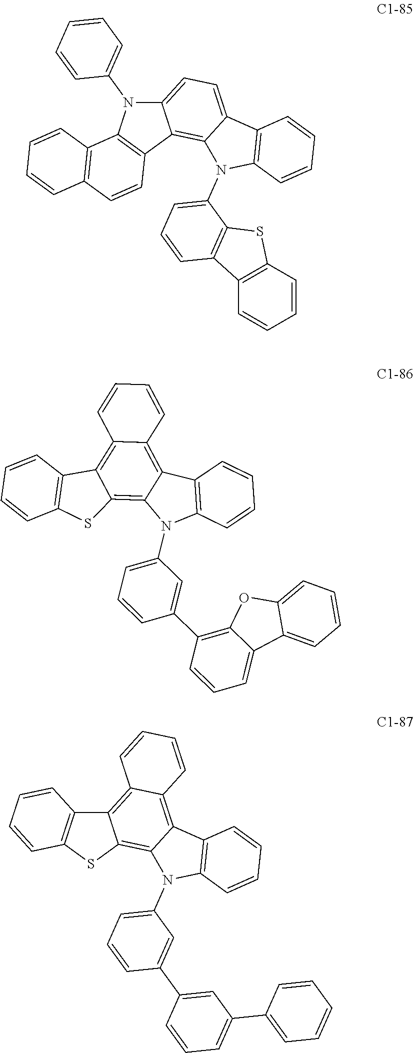 [SMILES: c1ccc(-n2c3ccc4c5ccccc5n(-c5cccc6c5sc5ccccc56)c4c3c3ccc4ccccc4c32)cc1 ; c1cc(-c2cccc3c2oc2ccccc23)cc(-n2c3ccccc3c3c4ccccc4c4c5ccccc5sc4c32)c1 ; c1ccc(-c2cccc(-c3cccc(-n4c5ccccc5c5c6ccccc6c6c7ccccc7sc6c54)c3)c2)cc1]

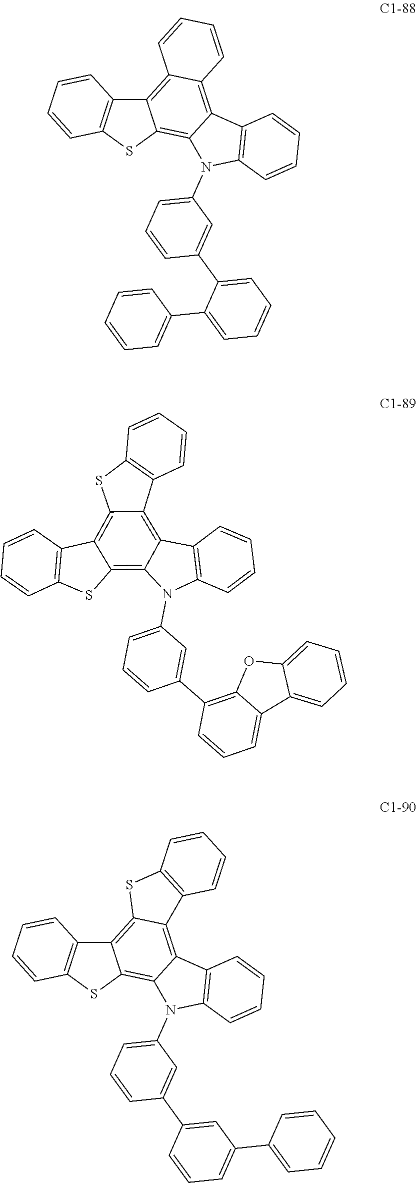 [SMILES: c1ccc(-c2ccccc2-c2cccc(-n3c4ccccc4c4c5ccccc5c5c6ccccc6sc5c43)c2)cc1 ; c1cc(-c2cccc3c2oc2ccccc23)cc(-n2c3ccccc3c3c4c5ccccc5sc4c4c5ccccc5sc4c32)c1 ; c1ccc(-c2cccc(-c3cccc(-n4c5ccccc5c5c6c7ccccc7sc6c6c7ccccc7sc6c54)c3)c2)cc1]

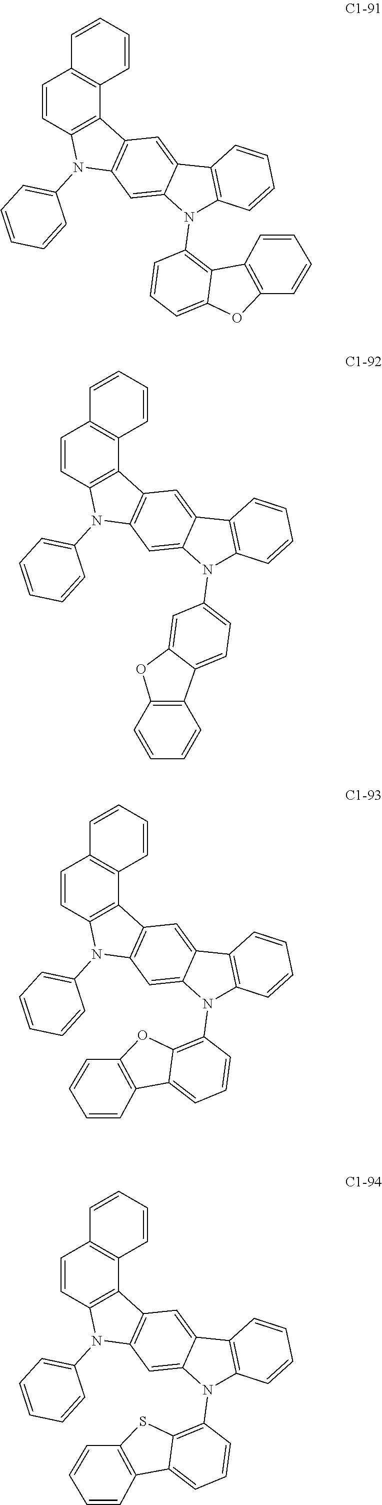 [SMILES: c1ccc(-n2c3cc4c(cc3c3c5ccccc5ccc32)c2ccccc2n4-c2cccc3oc4ccccc4c23)cc1 ; c1ccc(-n2c3cc4c(cc3c3c5ccccc5ccc32)c2ccccc2n4-c2ccc3c(c2)oc2ccccc23)cc1 ; c1ccc(-n2c3cc4c(cc3c3c5ccccc5ccc32)c2ccccc2n4-c2cccc3c2oc2ccccc23)cc1 ; c1ccc(-n2c3cc4c(cc3c3c5ccccc5ccc32)c2ccccc2n4-c2cccc3c2sc2ccccc23)cc1]

The compound represented by formula 2 includes the following compounds, but is not limited thereto.

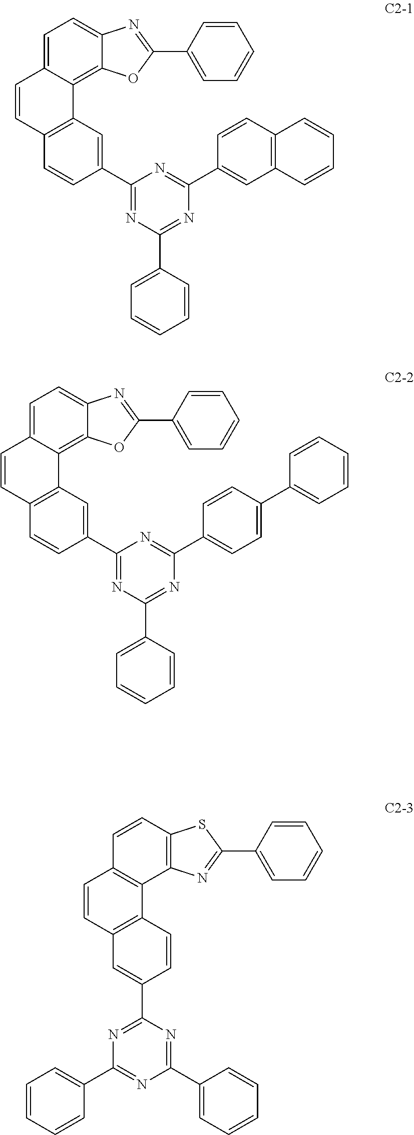 [SMILES: c1ccc(-c2nc(-c3ccc4ccccc4c3)nc(-c3ccc4ccc5ccc6nc(-c7ccccc7)oc6c5c4c3)n2)cc1 ; c1ccc(-c2ccc(-c3nc(-c4ccccc4)nc(-c4ccc5ccc6ccc7nc(-c8ccccc8)oc7c6c5c4)n3)cc2)cc1 ; c1ccc(-c2nc(-c3ccccc3)nc(-c3ccc4c(ccc5ccc6sc(-c7ccccc7)nc6c54)c3)n2)cc1]

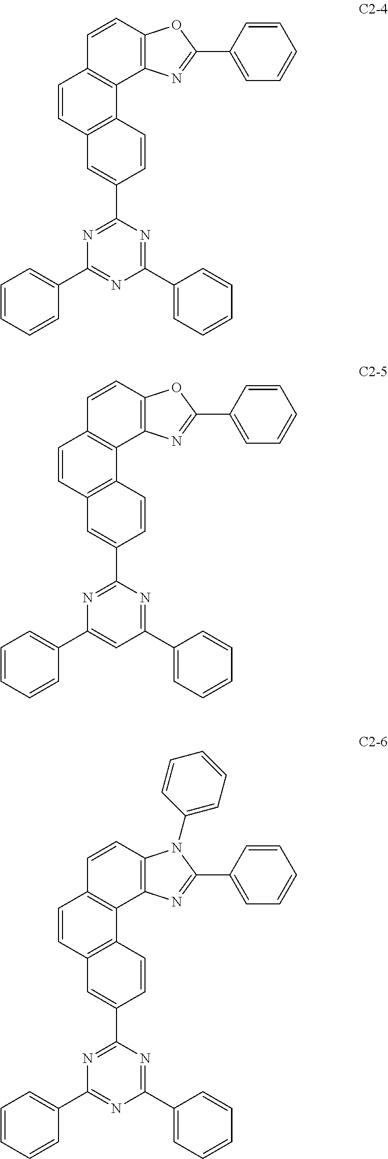 [SMILES: c1ccc(-c2nc(-c3ccccc3)nc(-c3ccc4c(ccc5ccc6oc(-c7ccccc7)nc6c54)c3)n2)cc1 ; c1ccc(-c2cc(-c3ccccc3)nc(-c3ccc4c(ccc5ccc6oc(-c7ccccc7)nc6c54)c3)n2)cc1 ; c1ccc(-c2nc(-c3ccccc3)nc(-c3ccc4c(ccc5ccc6c(nc(-c7ccccc7)n6-c6ccccc6)c54)c3)n2)cc1]

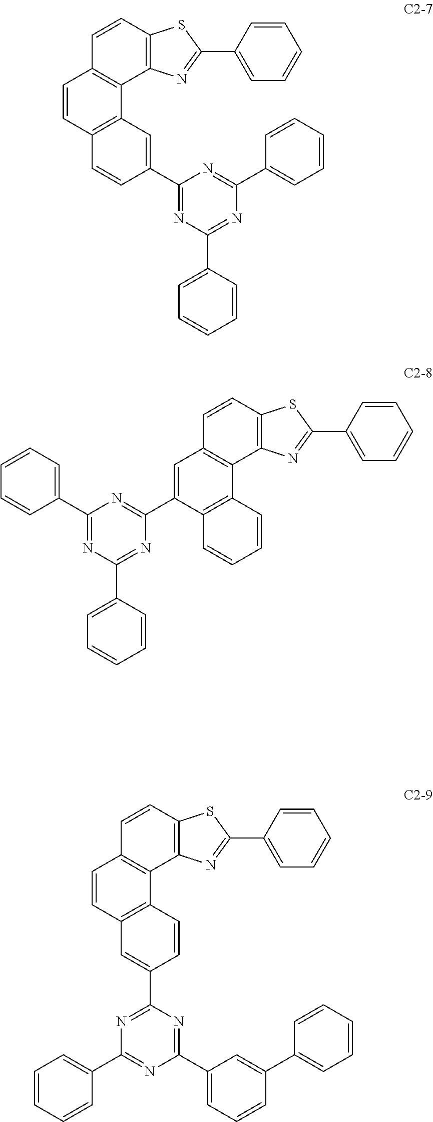 [SMILES: c1ccc(-c2nc(-c3ccccc3)nc(-c3ccc4ccc5ccc6sc(-c7ccccc7)nc6c5c4c3)n2)cc1 ; c1ccc(-c2nc(-c3ccccc3)nc(-c3cc4ccc5sc(-c6ccccc6)nc5c4c4ccccc34)n2)cc1 ; c1ccc(-c2cccc(-c3nc(-c4ccccc4)nc(-c4ccc5c(ccc6ccc7sc(-c8ccccc8)nc7c65)c4)n3)c2)cc1]

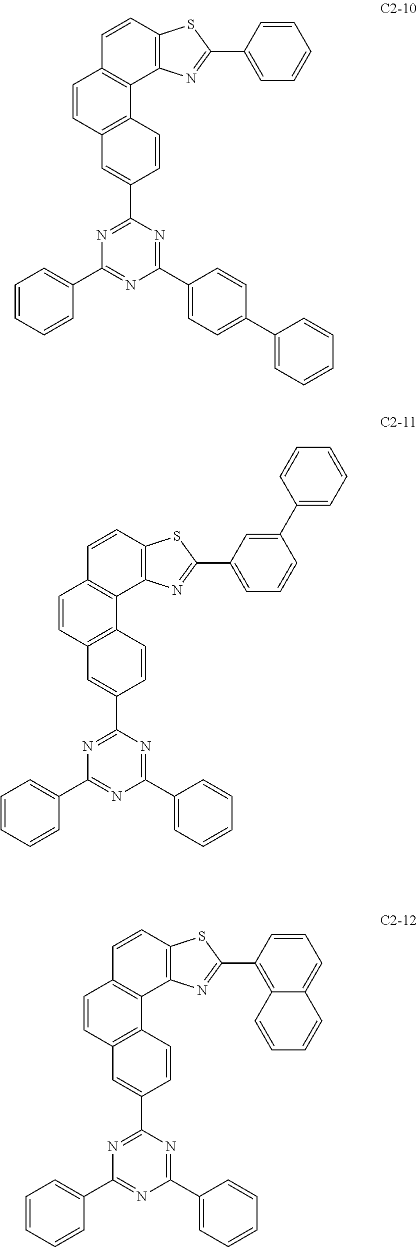 [SMILES: c1ccc(-c2ccc(-c3nc(-c4ccccc4)nc(-c4ccc5c(ccc6ccc7sc(-c8ccccc8)nc7c65)c4)n3)cc2)cc1 ; c1ccc(-c2cccc(-c3nc4c(ccc5ccc6cc(-c7nc(-c8ccccc8)nc(-c8ccccc8)n7)ccc6c54)s3)c2)cc1 ; c1ccc(-c2nc(-c3ccccc3)nc(-c3ccc4c(ccc5ccc6sc(-c7cccc8ccccc78)nc6c54)c3)n2)cc1]

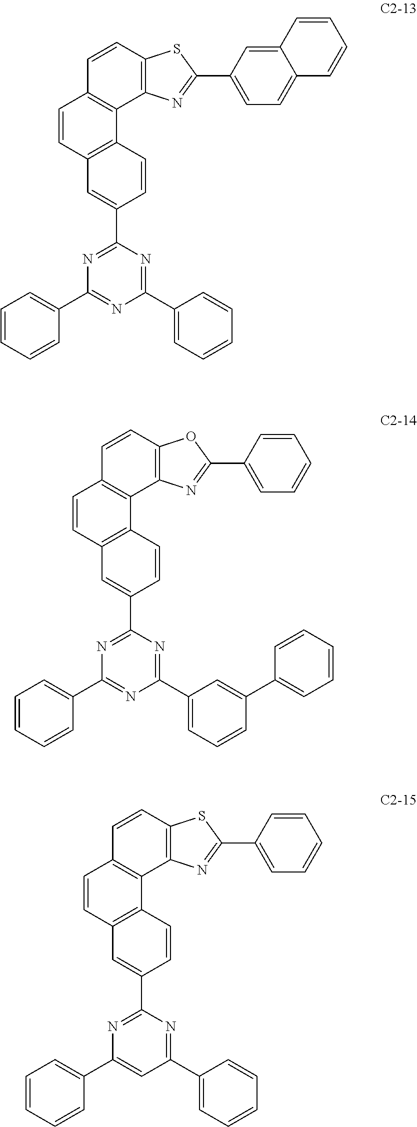 [SMILES: c1ccc(-c2nc(-c3ccccc3)nc(-c3ccc4c(ccc5ccc6sc(-c7ccc8ccccc8c7)nc6c54)c3)n2)cc1 ; c1ccc(-c2cccc(-c3nc(-c4ccccc4)nc(-c4ccc5c(ccc6ccc7oc(-c8ccccc8)nc7c65)c4)n3)c2)cc1 ; c1ccc(-c2cc(-c3ccccc3)nc(-c3ccc4c(ccc5ccc6sc(-c7ccccc7)nc6c54)c3)n2)cc1]

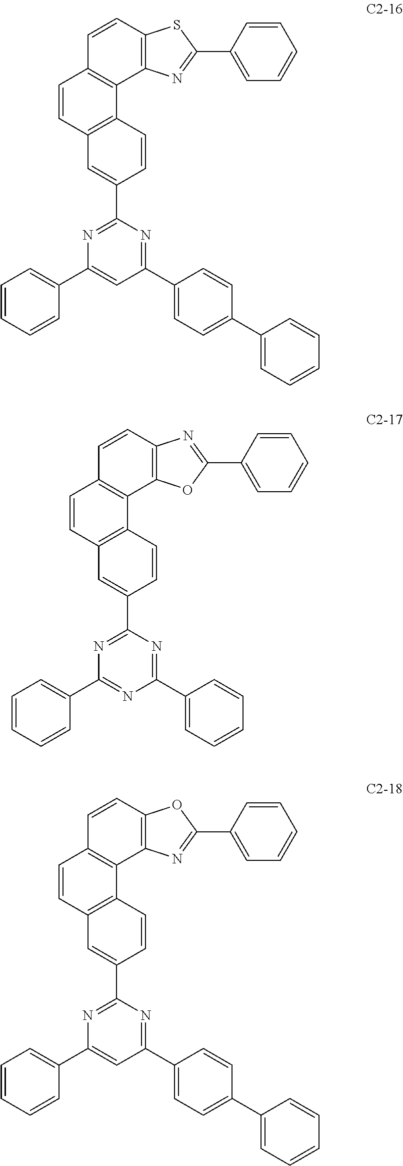 [SMILES: c1ccc(-c2ccc(-c3cc(-c4ccccc4)nc(-c4ccc5c(ccc6ccc7sc(-c8ccccc8)nc7c65)c4)n3)cc2)cc1 ; c1ccc(-c2nc(-c3ccccc3)nc(-c3ccc4c(ccc5ccc6nc(-c7ccccc7)oc6c54)c3)n2)cc1 ; c1ccc(-c2ccc(-c3cc(-c4ccccc4)nc(-c4ccc5c(ccc6ccc7oc(-c8ccccc8)nc7c65)c4)n3)cc2)cc1]

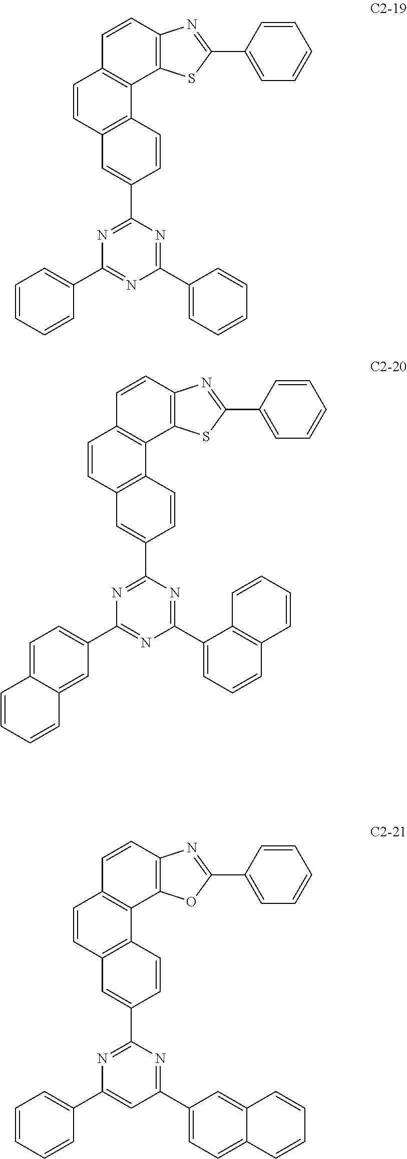 [SMILES: c1ccc(-c2nc(-c3ccccc3)nc(-c3ccc4c(ccc5ccc6nc(-c7ccccc7)sc6c54)c3)n2)cc1 ; c1ccc(-c2nc3ccc4ccc5cc(-c6nc(-c7ccc8ccccc8c7)nc(-c7cccc8ccccc78)n6)ccc5c4c3s2)cc1 ; c1ccc(-c2cc(-c3ccc4ccccc4c3)nc(-c3ccc4c(ccc5ccc6nc(-c7ccccc7)oc6c54)c3)n2)cc1]

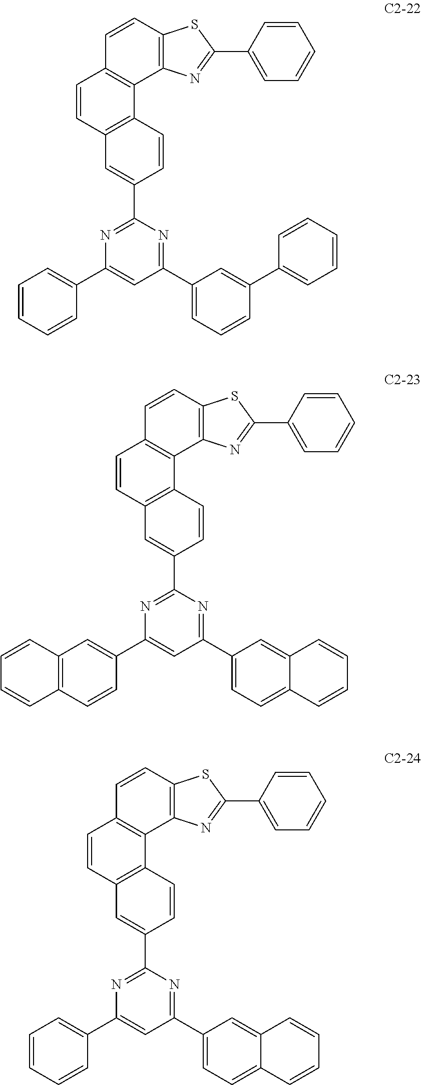 [SMILES: c1ccc(-c2cccc(-c3cc(-c4ccccc4)nc(-c4ccc5c(ccc6ccc7sc(-c8ccccc8)nc7c65)c4)n3)c2)cc1 ; c1ccc(-c2nc3c(ccc4ccc5cc(-c6nc(-c7ccc8ccccc8c7)cc(-c7ccc8ccccc8c7)n6)ccc5c43)s2)cc1 ; c1ccc(-c2cc(-c3ccc4ccccc4c3)nc(-c3ccc4c(ccc5ccc6sc(-c7ccccc7)nc6c54)c3)n2)cc1]

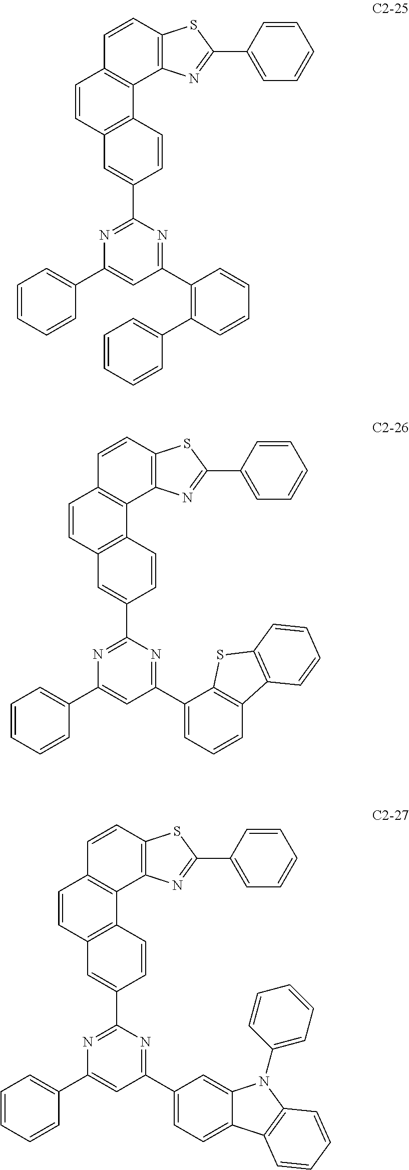 [SMILES: c1ccc(-c2cc(-c3ccccc3-c3ccccc3)nc(-c3ccc4c(ccc5ccc6sc(-c7ccccc7)nc6c54)c3)n2)cc1 ; c1ccc(-c2cc(-c3cccc4c3sc3ccccc34)nc(-c3ccc4c(ccc5ccc6sc(-c7ccccc7)nc6c54)c3)n2)cc1 ; c1ccc(-c2cc(-c3ccc4c5ccccc5n(-c5ccccc5)c4c3)nc(-c3ccc4c(ccc5ccc6sc(-c7ccccc7)nc6c54)c3)n2)cc1]

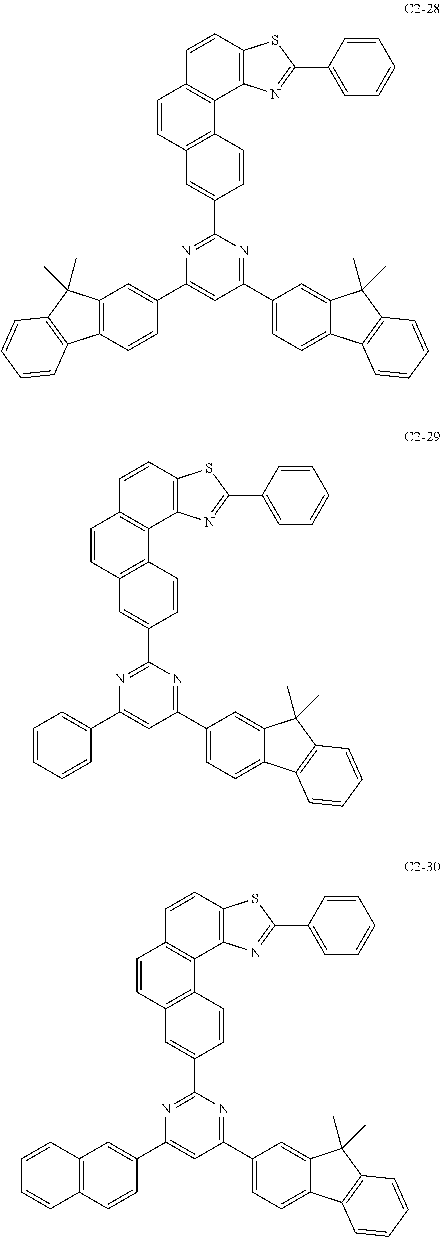 [SMILES: CC1(C)c2ccccc2-c2ccc(-c3cc(-c4ccc5c(c4)C(C)(C)c4ccccc4-5)nc(-c4ccc5c(ccc6ccc7sc(-c8ccccc8)nc7c65)c4)n3)cc21 ; CC1(C)c2ccccc2-c2ccc(-c3cc(-c4ccccc4)nc(-c4ccc5c(ccc6ccc7sc(-c8ccccc8)nc7c65)c4)n3)cc21 ; CC1(C)c2ccccc2-c2ccc(-c3cc(-c4ccc5ccccc5c4)nc(-c4ccc5c(ccc6ccc7sc(-c8ccccc8)nc7c65)c4)n3)cc21]

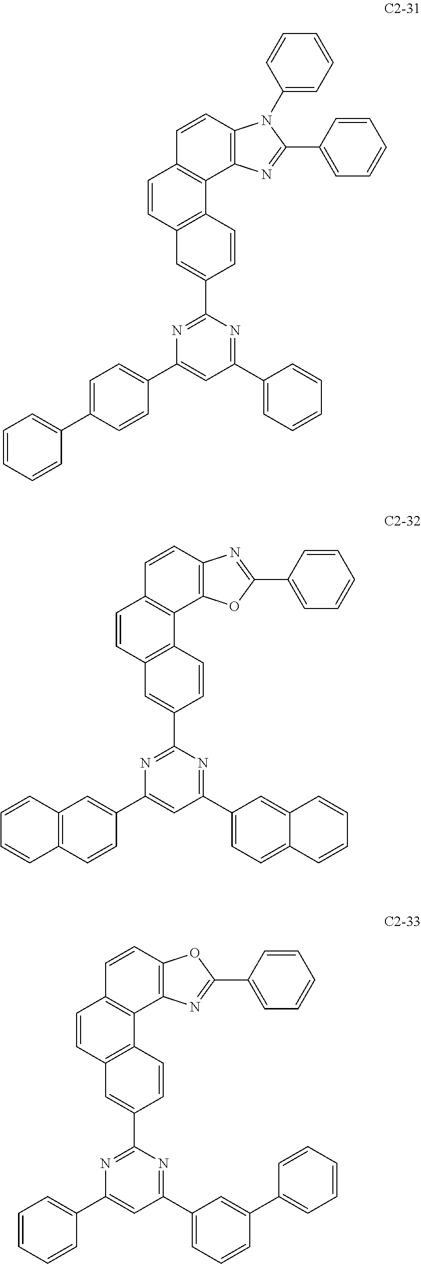 [SMILES: c1ccc(-c2ccc(-c3cc(-c4ccccc4)nc(-c4ccc5c(ccc6ccc7c(nc(-c8ccccc8)n7-c7ccccc7)c65)c4)n3)cc2)cc1 ; c1ccc(-c2nc3ccc4ccc5cc(-c6nc(-c7ccc8ccccc8c7)cc(-c7ccc8ccccc8c7)n6)ccc5c4c3o2)cc1 ; c1ccc(-c2cccc(-c3cc(-c4ccccc4)nc(-c4ccc5c(ccc6ccc7oc(-c8ccccc8)nc7c65)c4)n3)c2)cc1]

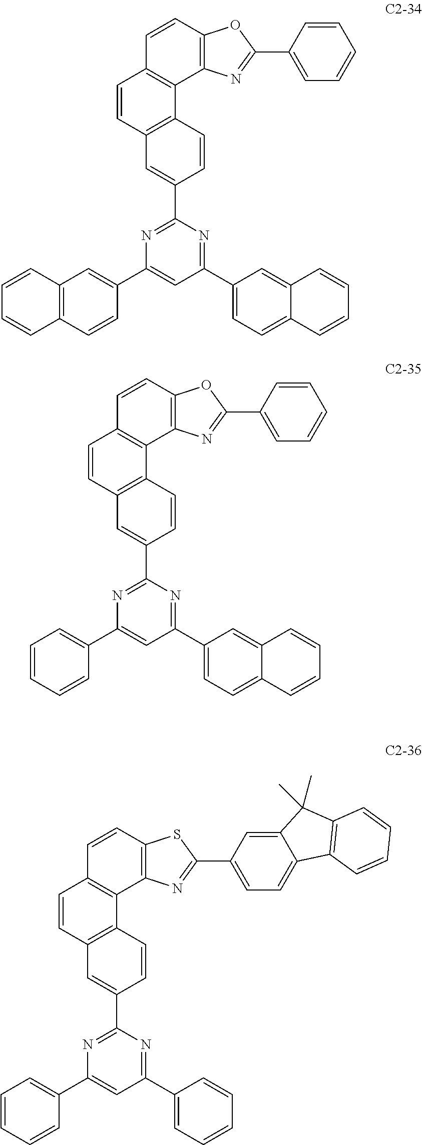 [SMILES: c1ccc(-c2nc3c(ccc4ccc5cc(-c6nc(-c7ccc8ccccc8c7)cc(-c7ccc8ccccc8c7)n6)ccc5c43)o2)cc1 ; c1ccc(-c2cc(-c3ccc4ccccc4c3)nc(-c3ccc4c(ccc5ccc6oc(-c7ccccc7)nc6c54)c3)n2)cc1 ; CC1(C)c2ccccc2-c2ccc(-c3nc4c(ccc5ccc6cc(-c7nc(-c8ccccc8)cc(-c8ccccc8)n7)ccc6c54)s3)cc21]

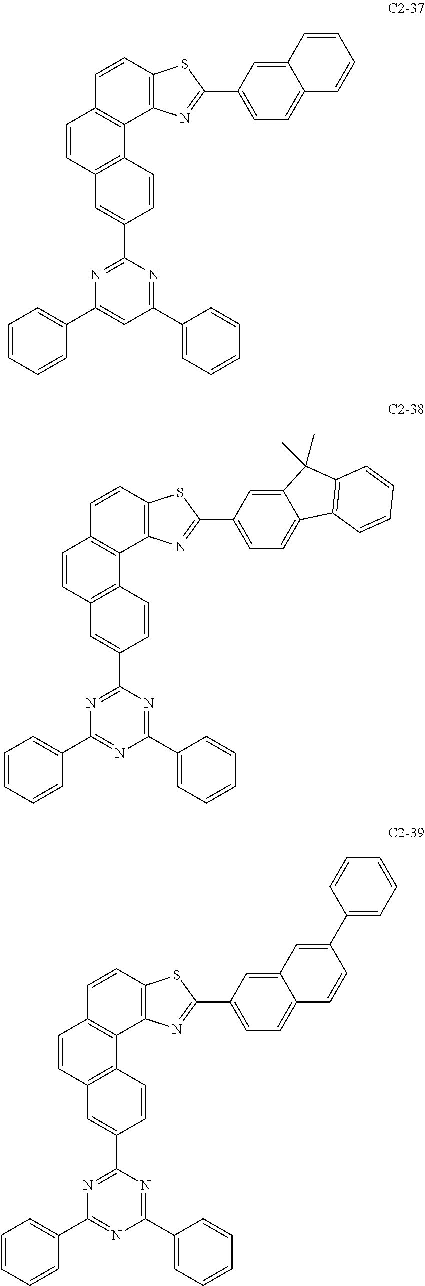 [SMILES: c1ccc(-c2cc(-c3ccccc3)nc(-c3ccc4c(ccc5ccc6sc(-c7ccc8ccccc8c7)nc6c54)c3)n2)cc1 ; CC1(C)c2ccccc2-c2ccc(-c3nc4c(ccc5ccc6cc(-c7nc(-c8ccccc8)nc(-c8ccccc8)n7)ccc6c54)s3)cc21 ; c1ccc(-c2ccc3ccc(-c4nc5c(ccc6ccc7cc(-c8nc(-c9ccccc9)nc(-c9ccccc9)n8)ccc7c65)s4)cc3c2)cc1]

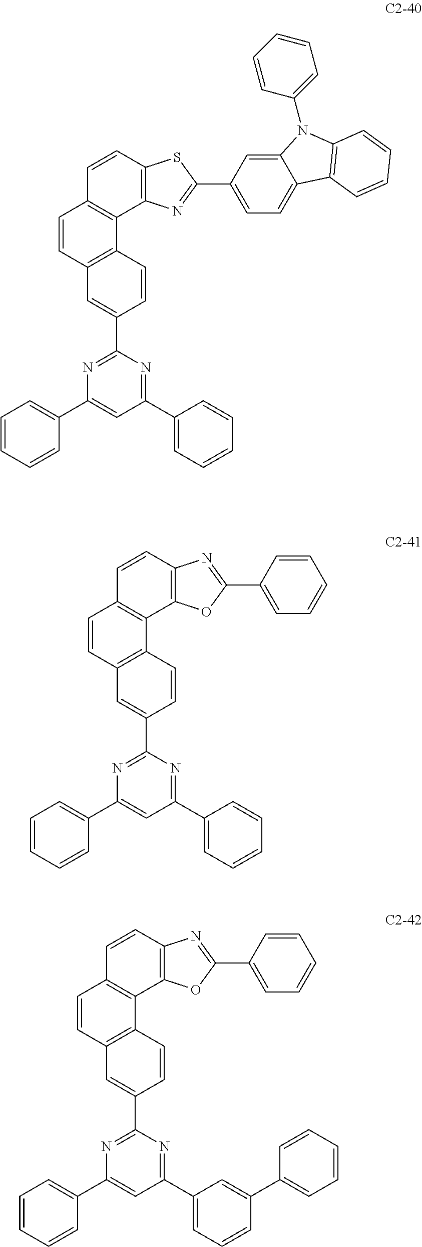 [SMILES: c1ccc(-c2cc(-c3ccccc3)nc(-c3ccc4c(ccc5ccc6sc(-c7ccc8c9ccccc9n(-c9ccccc9)c8c7)nc6c54)c3)n2)cc1 ; c1ccc(-c2cc(-c3ccccc3)nc(-c3ccc4c(ccc5ccc6nc(-c7ccccc7)oc6c54)c3)n2)cc1 ; c1ccc(-c2cccc(-c3cc(-c4ccccc4)nc(-c4ccc5c(ccc6ccc7nc(-c8ccccc8)oc7c65)c4)n3)c2)cc1]

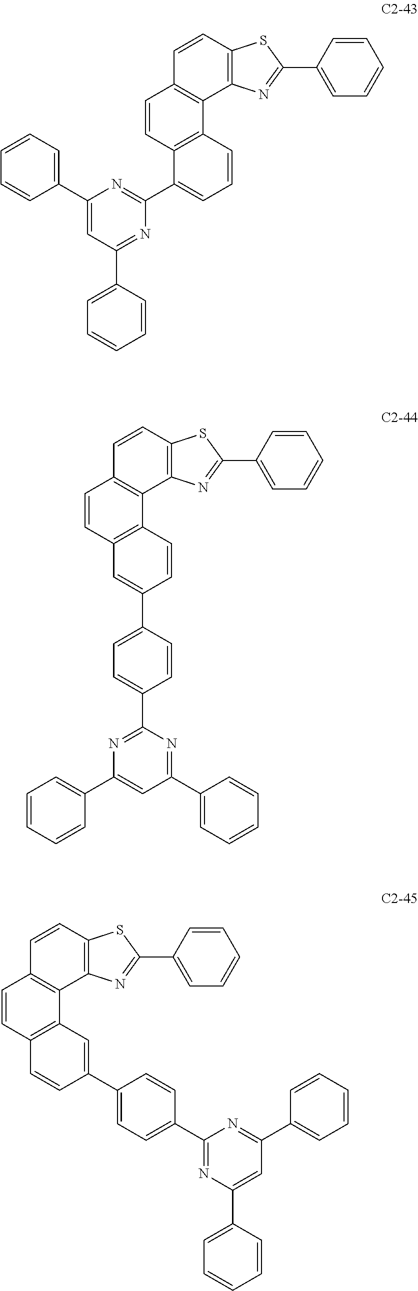 [SMILES: c1ccc(-c2cc(-c3ccccc3)nc(-c3cccc4c3ccc3ccc5sc(-c6ccccc6)nc5c34)n2)cc1 ; c1ccc(-c2cc(-c3ccccc3)nc(-c3ccc(-c4ccc5c(ccc6ccc7sc(-c8ccccc8)nc7c65)c4)cc3)n2)cc1 ; c1ccc(-c2cc(-c3ccccc3)nc(-c3ccc(-c4ccc5ccc6ccc7sc(-c8ccccc8)nc7c6c5c4)cc3)n2)cc1]

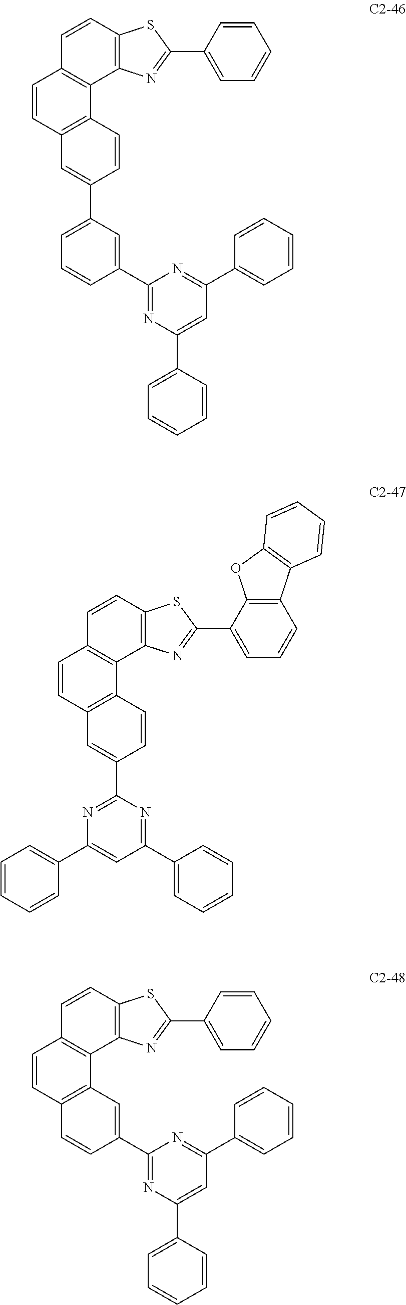 [SMILES: c1ccc(-c2cc(-c3ccccc3)nc(-c3cccc(-c4ccc5c(ccc6ccc7sc(-c8ccccc8)nc7c65)c4)c3)n2)cc1 ; c1ccc(-c2cc(-c3ccccc3)nc(-c3ccc4c(ccc5ccc6sc(-c7cccc8c7oc7ccccc78)nc6c54)c3)n2)cc1 ; c1ccc(-c2cc(-c3ccccc3)nc(-c3ccc4ccc5ccc6sc(-c7ccccc7)nc6c5c4c3)n2)cc1]

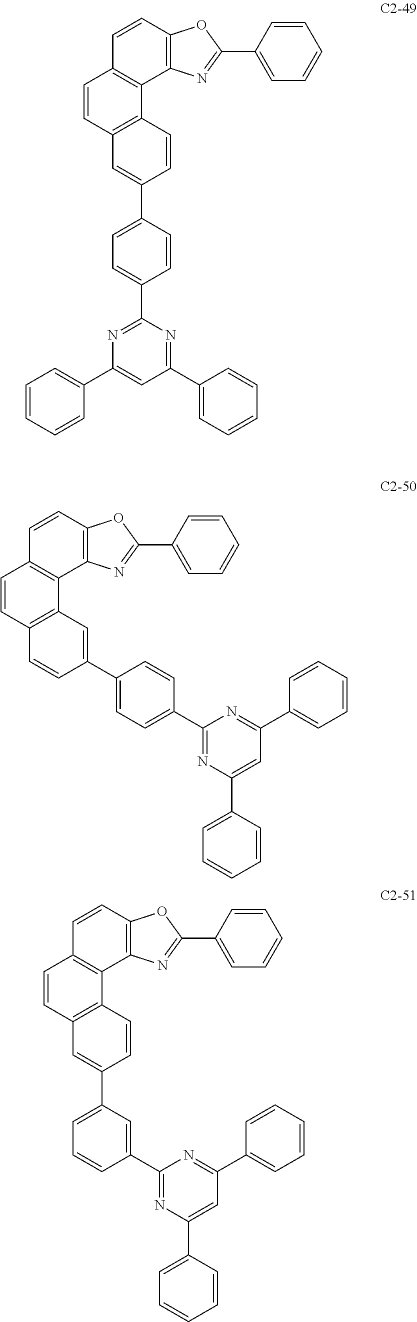

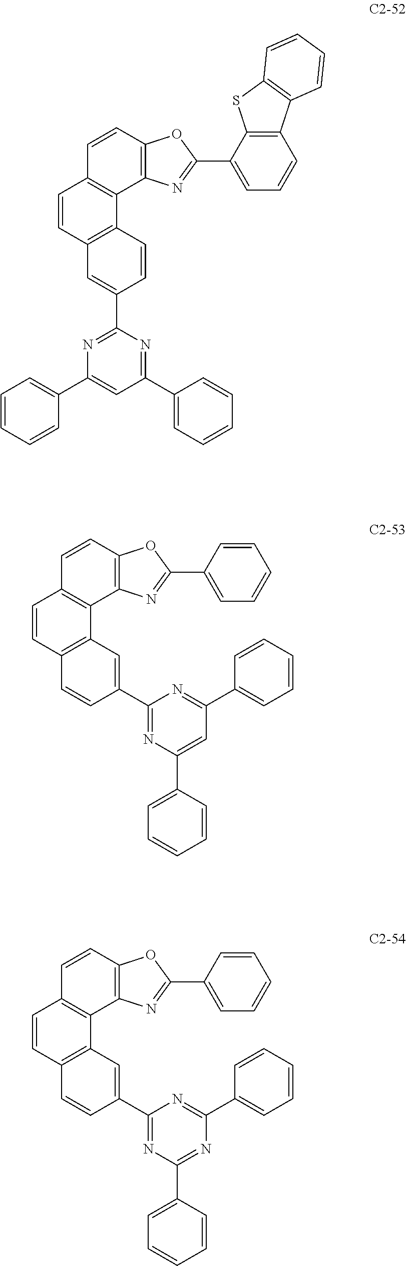 [SMILES: c1ccc(-c2cc(-c3ccccc3)nc(-c3ccc4c(ccc5ccc6oc(-c7cccc8c7sc7ccccc78)nc6c54)c3)n2)cc1 ; c1ccc(-c2cc(-c3ccccc3)nc(-c3ccc4ccc5ccc6oc(-c7ccccc7)nc6c5c4c3)n2)cc1 ; c1ccc(-c2nc(-c3ccccc3)nc(-c3ccc4ccc5ccc6oc(-c7ccccc7)nc6c5c4c3)n2)cc1]

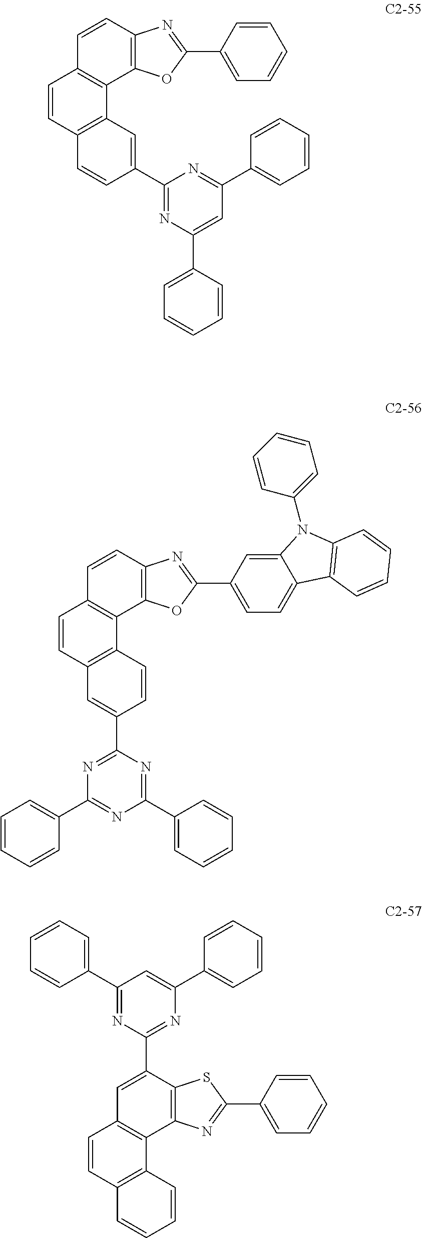 [SMILES: c1ccc(-c2cc(-c3ccccc3)nc(-c3ccc4ccc5ccc6nc(-c7ccccc7)oc6c5c4c3)n2)cc1 ; c1ccc(-c2nc(-c3ccccc3)nc(-c3ccc4c(ccc5ccc6nc(-c7ccc8c9ccccc9n(-c9ccccc9)c8c7)oc6c54)c3)n2)cc1 ; c1ccc(-c2cc(-c3ccccc3)nc(-c3cc4ccc5ccccc5c4c4nc(-c5ccccc5)sc34)n2)cc1]

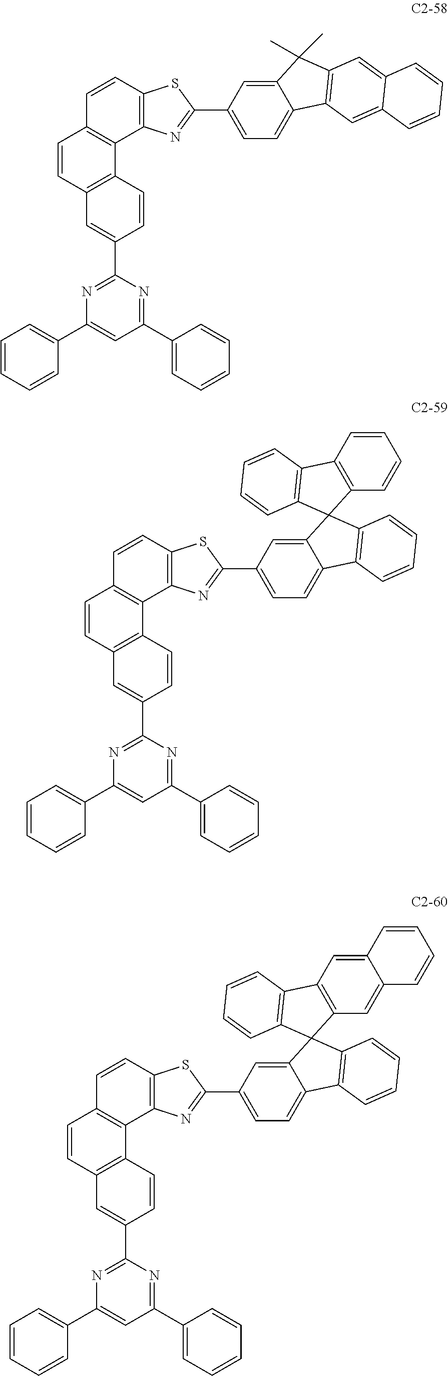 [SMILES: CC1(C)c2cc(-c3nc4c(ccc5ccc6cc(-c7nc(-c8ccccc8)cc(-c8ccccc8)n7)ccc6c54)s3)ccc2-c2cc3ccccc3cc21 ; c1ccc(-c2cc(-c3ccccc3)nc(-c3ccc4c(ccc5ccc6sc(-c7ccc8c(c7)C7(c9ccccc9-c9ccccc97)c7ccccc7-8)nc6c54)c3)n2)cc1 ; c1ccc(-c2cc(-c3ccccc3)nc(-c3ccc4c(ccc5ccc6sc(-c7ccc8c(c7)C7(c9ccccc9-8)c8ccccc8-c8cc9ccccc9cc87)nc6c54)c3)n2)cc1]

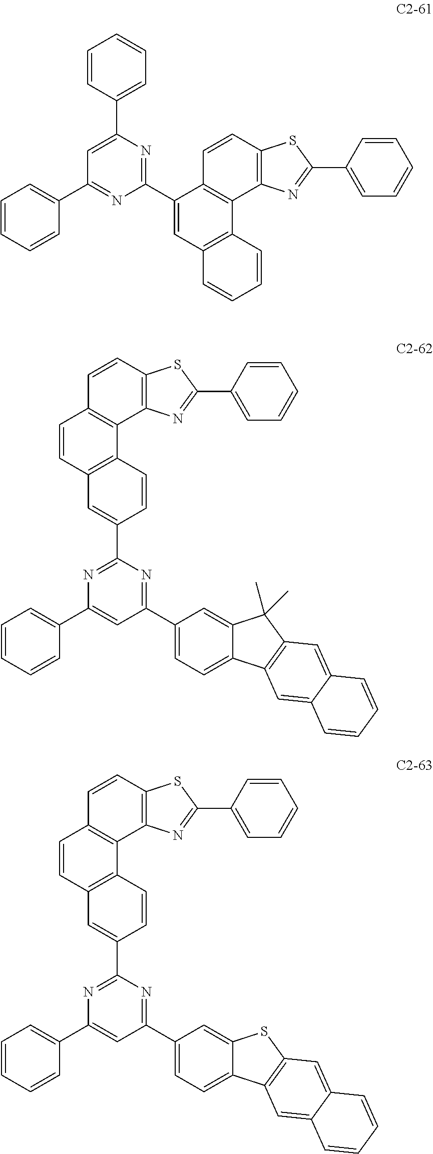 [SMILES: c1ccc(-c2cc(-c3ccccc3)nc(-c3cc4ccccc4c4c3ccc3sc(-c5ccccc5)nc34)n2)cc1 ; CC1(C)c2cc(-c3cc(-c4ccccc4)nc(-c4ccc5c(ccc6ccc7sc(-c8ccccc8)nc7c65)c4)n3)ccc2-c2cc3ccccc3cc21 ; c1ccc(-c2cc(-c3ccc4c(c3)sc3cc5ccccc5cc34)nc(-c3ccc4c(ccc5ccc6sc(-c7ccccc7)nc6c54)c3)n2)cc1]

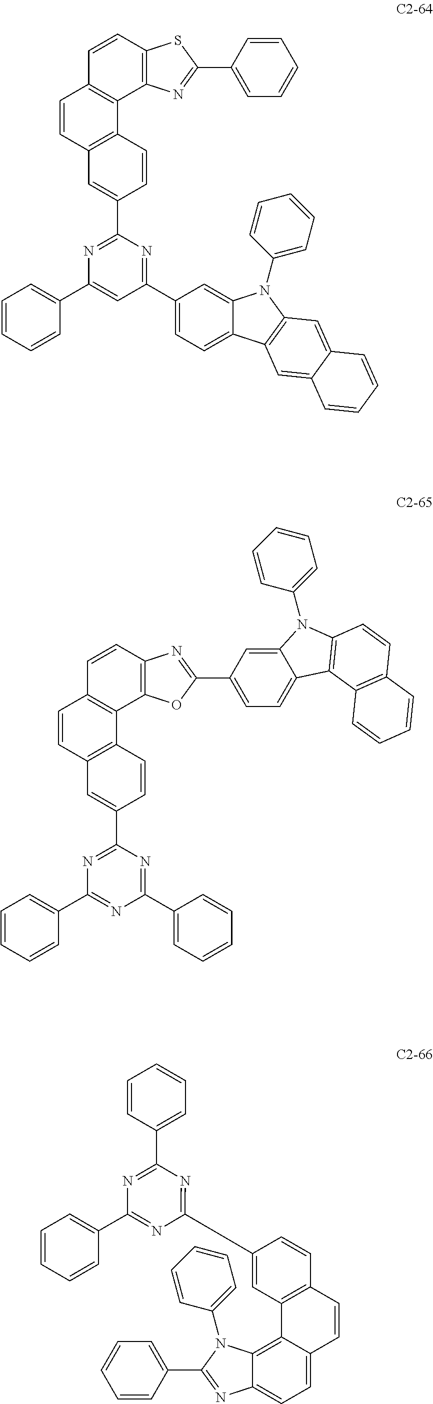 [SMILES: c1ccc(-c2cc(-c3ccc4c5cc6ccccc6cc5n(-c5ccccc5)c4c3)nc(-c3ccc4c(ccc5ccc6sc(-c7ccccc7)nc6c54)c3)n2)cc1 ; c1ccc(-c2nc(-c3ccccc3)nc(-c3ccc4c(ccc5ccc6nc(-c7ccc8c9c%10ccccc%10ccc9n(-c9ccccc9)c8c7)oc6c54)c3)n2)cc1 ; c1ccc(-c2nc(-c3ccccc3)nc(-c3ccc4ccc5ccc6nc(-c7ccccc7)n(-c7ccccc7)c6c5c4c3)n2)cc1]

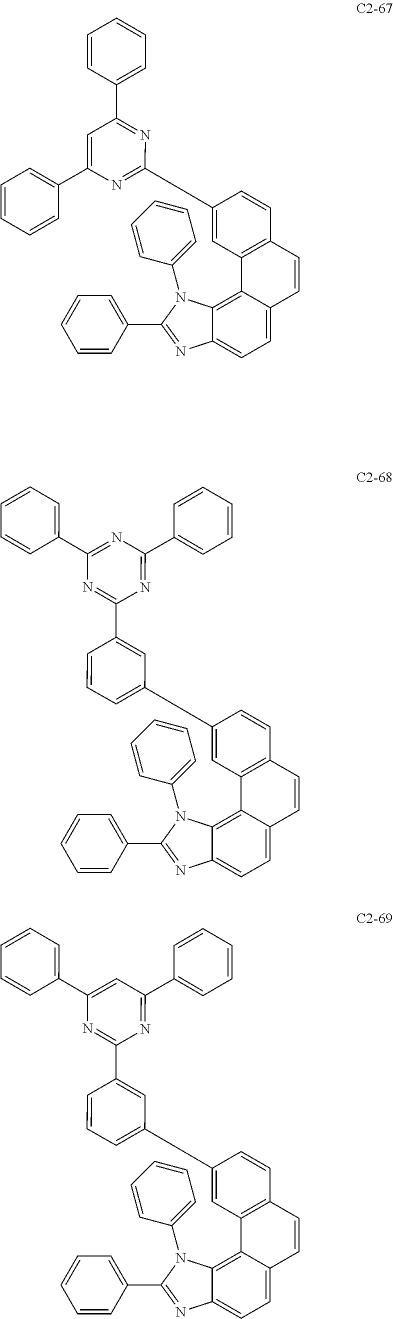 [SMILES: c1ccc(-c2cc(-c3ccccc3)nc(-c3ccc4ccc5ccc6nc(-c7ccccc7)n(-c7ccccc7)c6c5c4c3)n2)cc1 ; c1ccc(-c2nc(-c3ccccc3)nc(-c3cccc(-c4ccc5ccc6ccc7nc(-c8ccccc8)n(-c8ccccc8)c7c6c5c4)c3)n2)cc1 ; c1ccc(-c2cc(-c3ccccc3)nc(-c3cccc(-c4ccc5ccc6ccc7nc(-c8ccccc8)n(-c8ccccc8)c7c6c5c4)c3)n2)cc1]

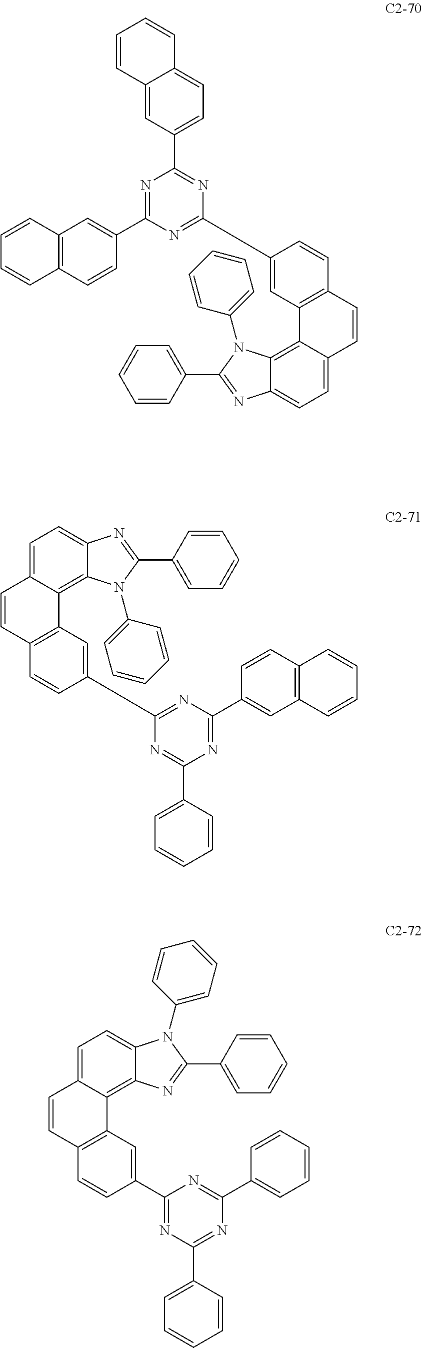 [SMILES: c1ccc(-c2nc3ccc4ccc5ccc(-c6nc(-c7ccc8ccccc8c7)nc(-c7ccc8ccccc8c7)n6)cc5c4c3n2-c2ccccc2)cc1 ; c1ccc(-c2nc(-c3ccc4ccccc4c3)nc(-c3ccc4ccc5ccc6nc(-c7ccccc7)n(-c7ccccc7)c6c5c4c3)n2)cc1 ; c1ccc(-c2nc(-c3ccccc3)nc(-c3ccc4ccc5ccc6c(nc(-c7ccccc7)n6-c6ccccc6)c5c4c3)n2)cc1]

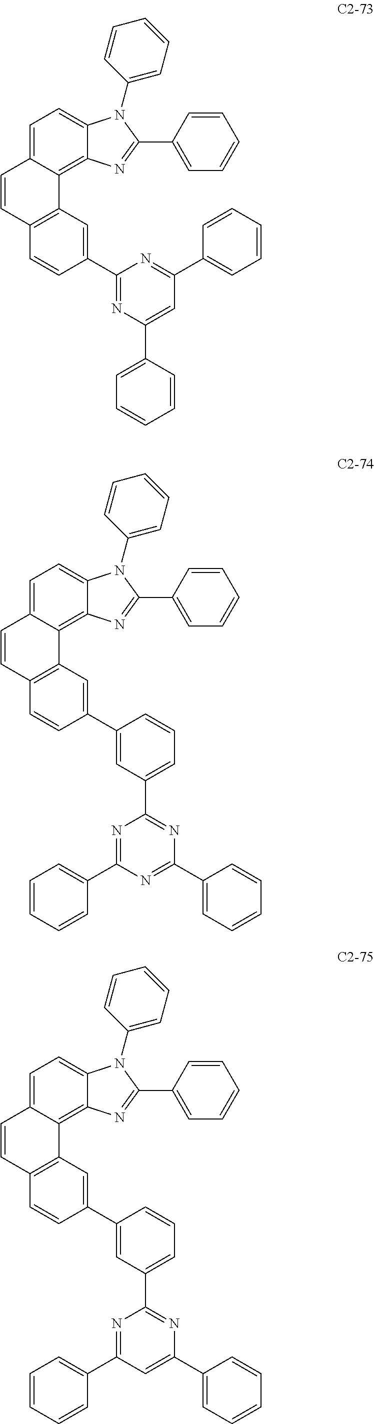 [SMILES: c1ccc(-c2cc(-c3ccccc3)nc(-c3ccc4ccc5ccc6c(nc(-c7ccccc7)n6-c6ccccc6)c5c4c3)n2)cc1 ; c1ccc(-c2nc(-c3ccccc3)nc(-c3cccc(-c4ccc5ccc6ccc7c(nc(-c8ccccc8)n7-c7ccccc7)c6c5c4)c3)n2)cc1 ; c1ccc(-c2cc(-c3ccccc3)nc(-c3cccc(-c4ccc5ccc6ccc7c(nc(-c8ccccc8)n7-c7ccccc7)c6c5c4)c3)n2)cc1]

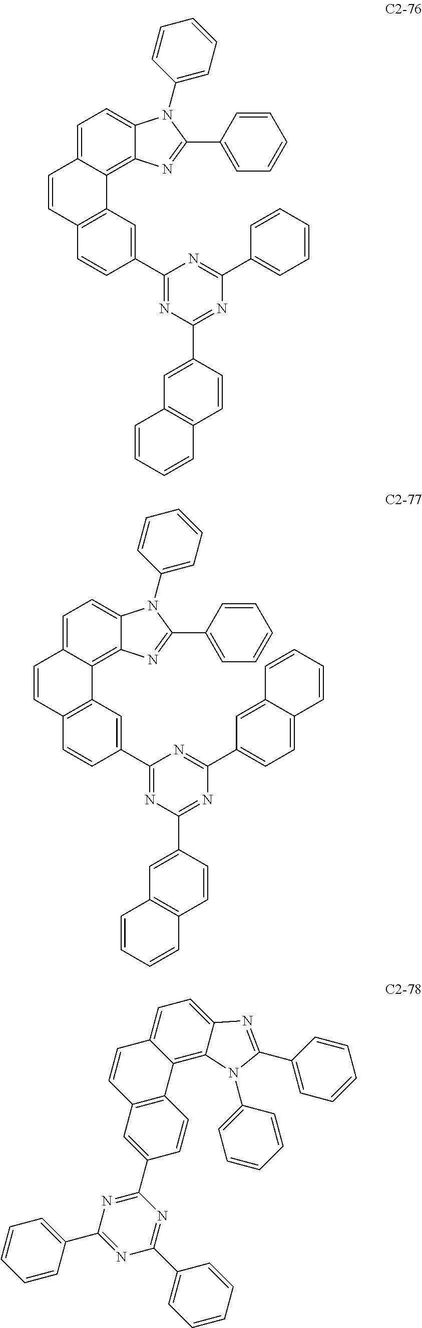

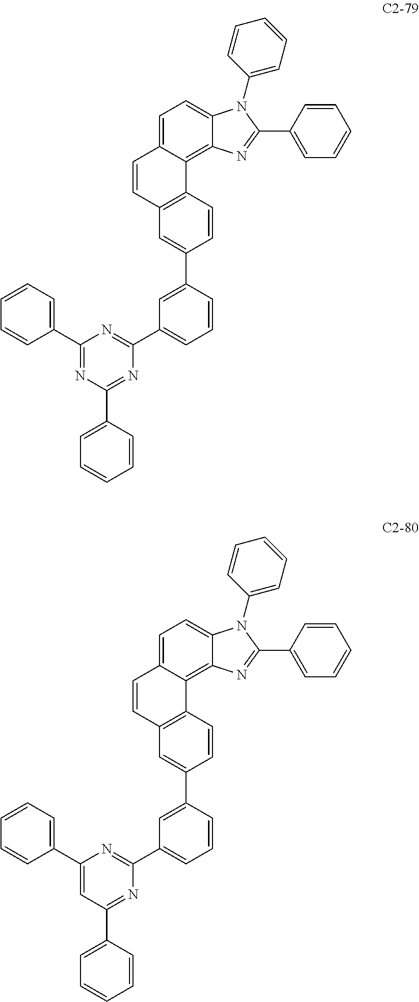 [SMILES: c1ccc(-c2nc(-c3ccccc3)nc(-c3cccc(-c4ccc5c(ccc6ccc7c(nc(-c8ccccc8)n7-c7ccccc7)c65)c4)c3)n2)cc1 ; c1ccc(-c2cc(-c3ccccc3)nc(-c3cccc(-c4ccc5c(ccc6ccc7c(nc(-c8ccccc8)n7-c7ccccc7)c65)c4)c3)n2)cc1]

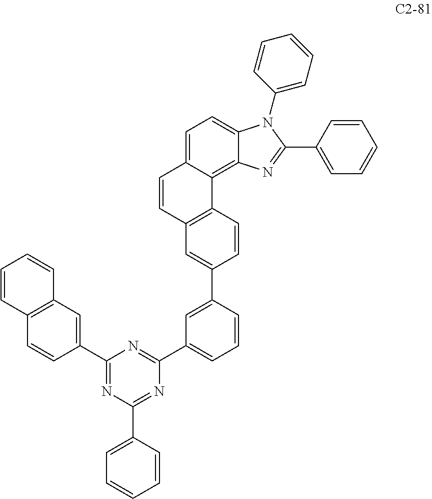 [SMILES: c1ccc(-c2nc(-c3cccc(-c4ccc5c(ccc6ccc7c(nc(-c8ccccc8)n7-c7ccccc7)c65)c4)c3)nc(-c3ccc4ccccc4c3)n2)cc1]

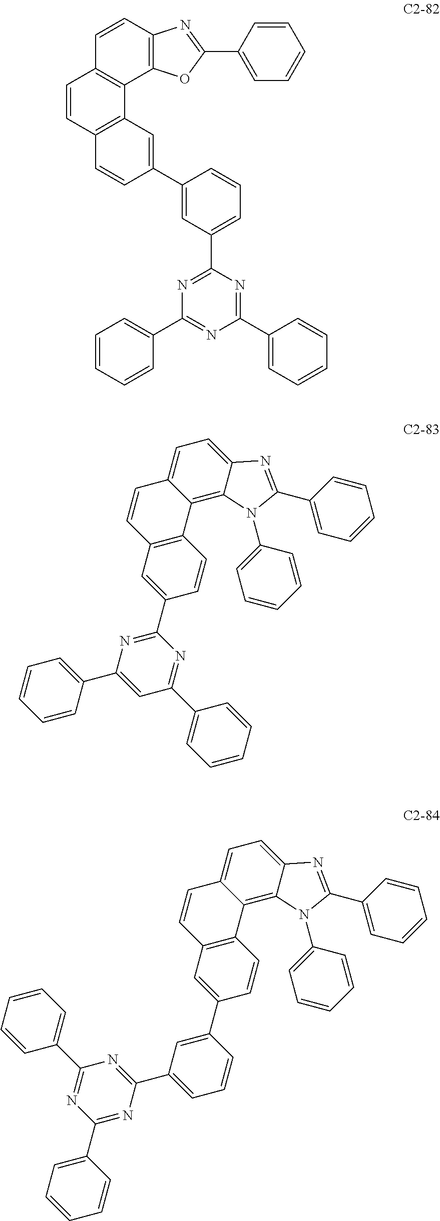 [SMILES: c1ccc(-c2nc(-c3ccccc3)nc(-c3cccc(-c4ccc5ccc6ccc7nc(-c8ccccc8)oc7c6c5c4)c3)n2)cc1 ; c1ccc(-c2cc(-c3ccccc3)nc(-c3ccc4c(ccc5ccc6nc(-c7ccccc7)n(-c7ccccc7)c6c54)c3)n2)cc1 ; c1ccc(-c2nc(-c3ccccc3)nc(-c3cccc(-c4ccc5c(ccc6ccc7nc(-c8ccccc8)n(-c8ccccc8)c7c65)c4)c3)n2)cc1]

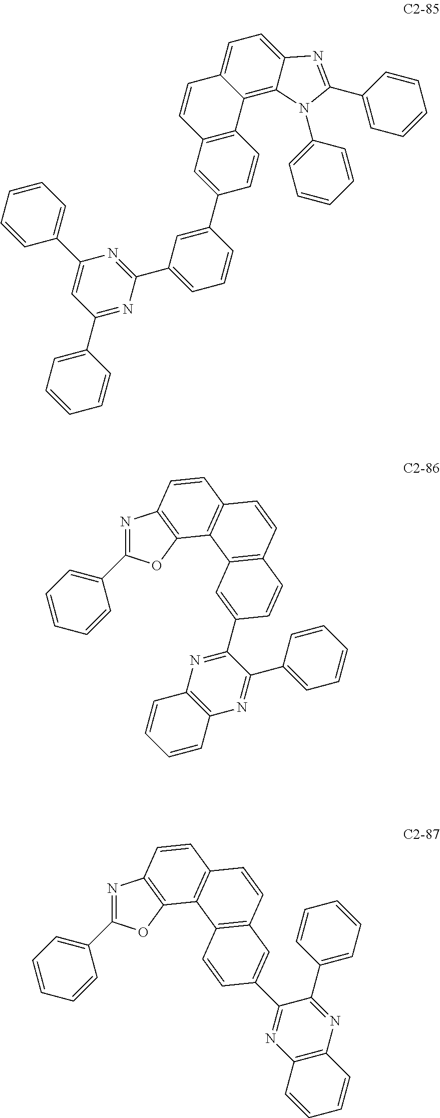 [SMILES: c1ccc(-c2cc(-c3ccccc3)nc(-c3cccc(-c4ccc5c(ccc6ccc7nc(-c8ccccc8)n(-c8ccccc8)c7c65)c4)c3)n2)cc1 ; c1ccc(-c2nc3ccc4ccc5ccc(-c6nc7ccccc7nc6-c6ccccc6)cc5c4c3o2)cc1 ; c1ccc(-c2nc3ccc4ccc5cc(-c6nc7ccccc7nc6-c6ccccc6)ccc5c4c3o2)cc1]

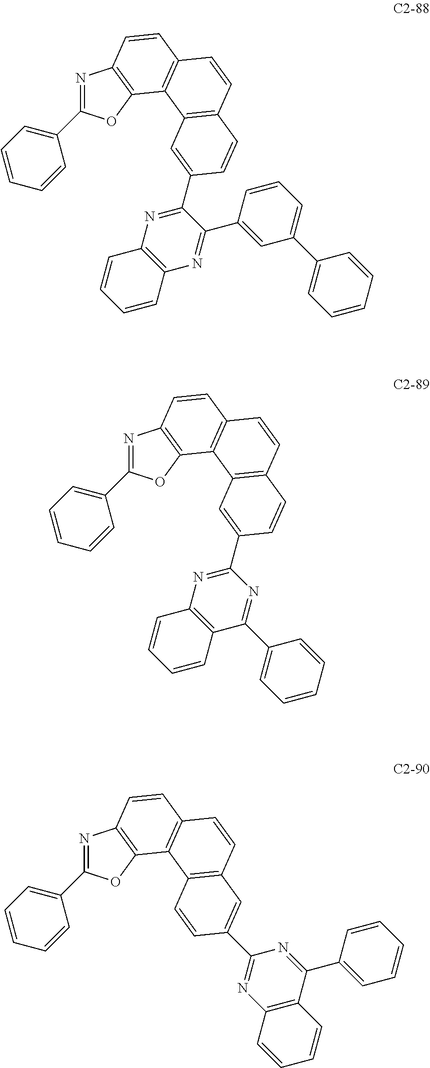 [SMILES: c1ccc(-c2cccc(-c3nc4ccccc4nc3-c3ccc4ccc5ccc6nc(-c7ccccc7)oc6c5c4c3)c2)cc1 ; c1ccc(-c2nc3ccc4ccc5ccc(-c6nc(-c7ccccc7)c7ccccc7n6)cc5c4c3o2)cc1 ; c1ccc(-c2nc3ccc4ccc5cc(-c6nc(-c7ccccc7)c7ccccc7n6)ccc5c4c3o2)cc1]

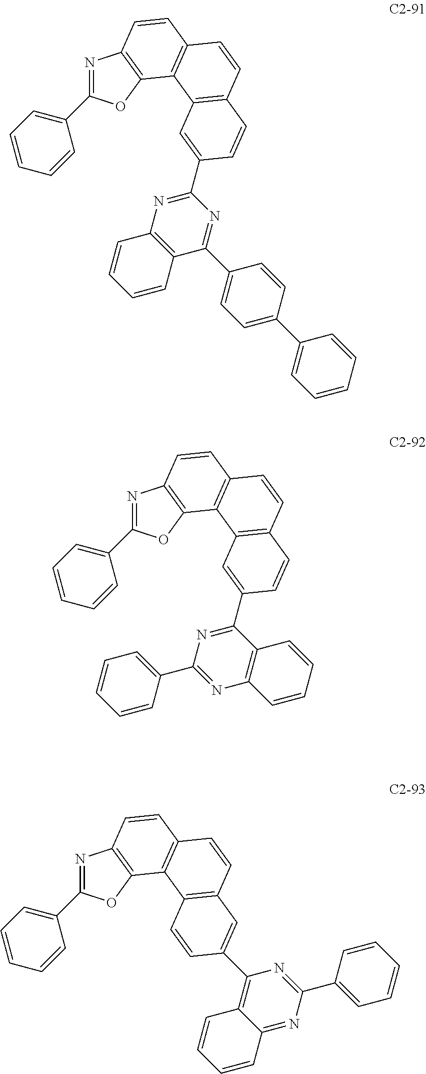 [SMILES: c1ccc(-c2ccc(-c3nc(-c4ccc5ccc6ccc7nc(-c8ccccc8)oc7c6c5c4)nc4ccccc34)cc2)cc1 ; c1ccc(-c2nc(-c3ccc4ccc5ccc6nc(-c7ccccc7)oc6c5c4c3)c3ccccc3n2)cc1 ; c1ccc(-c2nc(-c3ccc4c(ccc5ccc6nc(-c7ccccc7)oc6c54)c3)c3ccccc3n2)cc1]

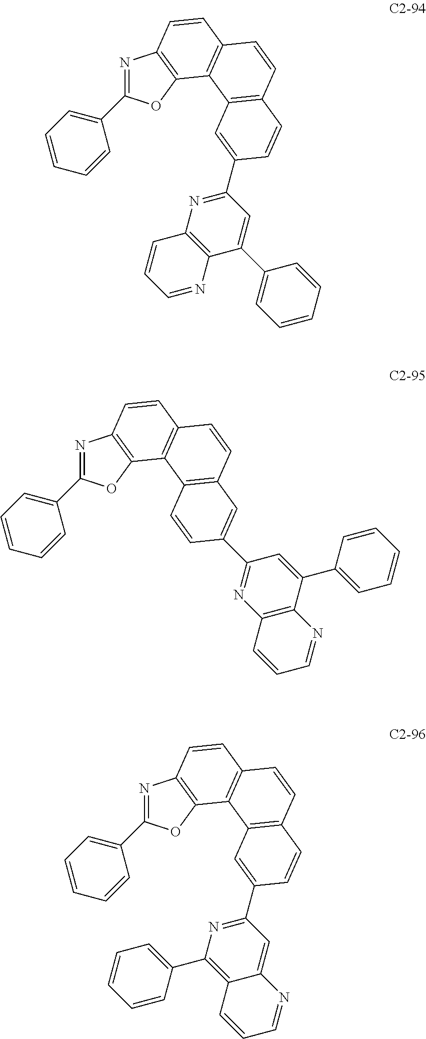 [SMILES: c1ccc(-c2nc3ccc4ccc5ccc(-c6cc(-c7ccccc7)c7ncccc7n6)cc5c4c3o2)cc1 ; c1ccc(-c2nc3ccc4ccc5cc(-c6cc(-c7ccccc7)c7ncccc7n6)ccc5c4c3o2)cc1 ; c1ccc(-c2nc3ccc4ccc5ccc(-c6cc7ncccc7c(-c7ccccc7)n6)cc5c4c3o2)cc1]

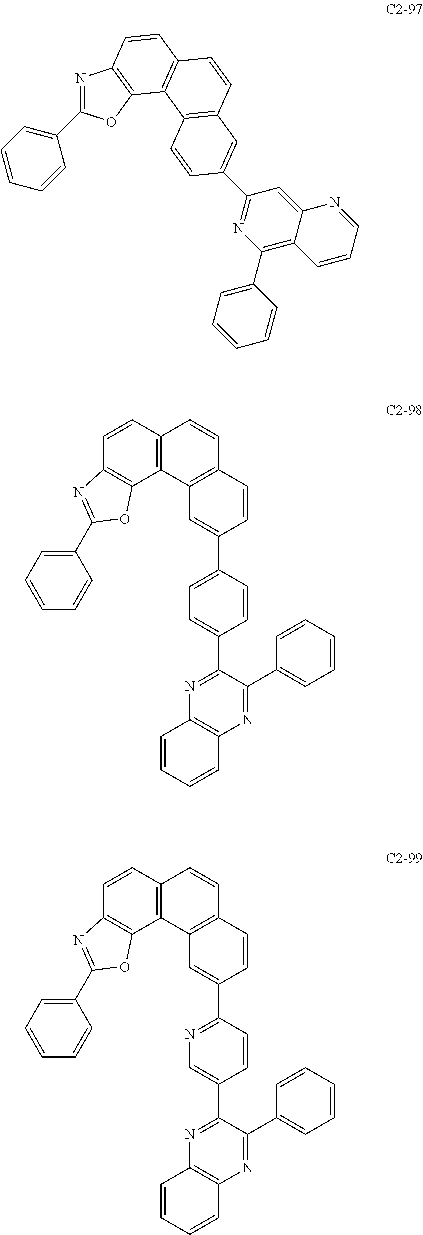 [SMILES: c1ccc(-c2nc3ccc4ccc5cc(-c6cc7ncccc7c(-c7ccccc7)n6)ccc5c4c3o2)cc1 ; c1ccc(-c2nc3ccc4ccc5ccc(-c6ccc(-c7nc8ccccc8nc7-c7ccccc7)cc6)cc5c4c3o2)cc1 ; c1ccc(-c2nc3ccc4ccc5ccc(-c6ccc(-c7nc8ccccc8nc7-c7ccccc7)cn6)cc5c4c3o2)cc1]

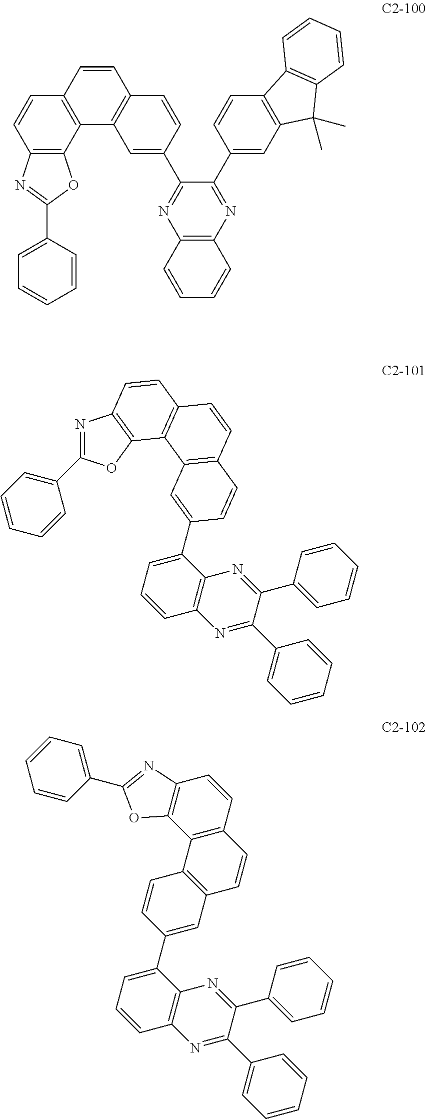 [SMILES: CC1(C)c2ccccc2-c2ccc(-c3nc4ccccc4nc3-c3ccc4ccc5ccc6nc(-c7ccccc7)oc6c5c4c3)cc21 ; c1ccc(-c2nc3ccc4ccc5ccc(-c6cccc7nc(-c8ccccc8)c(-c8ccccc8)nc67)cc5c4c3o2)cc1 ; c1ccc(-c2nc3ccc4ccc5cc(-c6cccc7nc(-c8ccccc8)c(-c8ccccc8)nc67)ccc5c4c3o2)cc1]

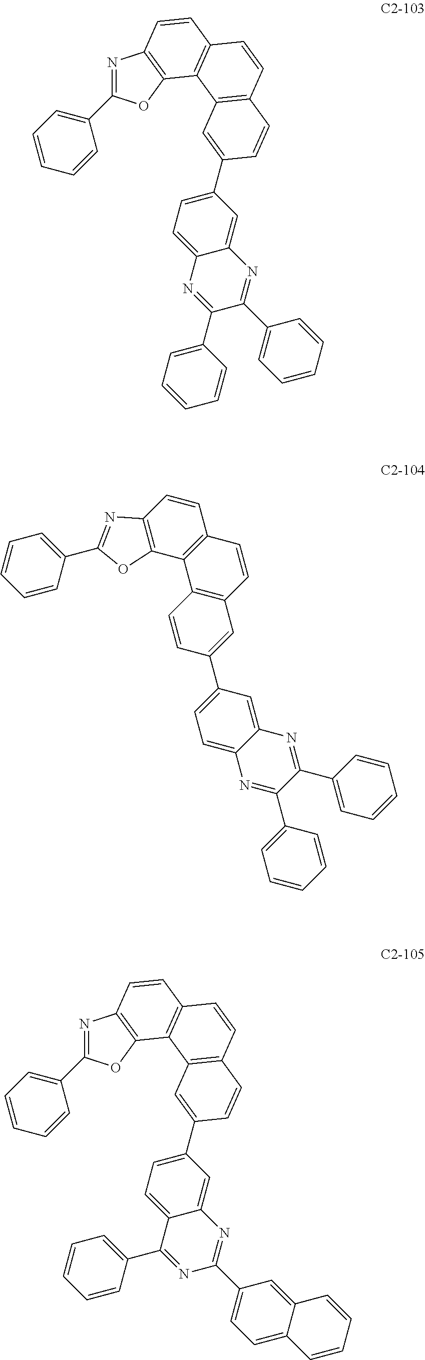 [SMILES: c1ccc(-c2nc3ccc4ccc5ccc(-c6ccc7nc(-c8ccccc8)c(-c8ccccc8)nc7c6)cc5c4c3o2)cc1 ; c1ccc(-c2nc3ccc4ccc5cc(-c6ccc7nc(-c8ccccc8)c(-c8ccccc8)nc7c6)ccc5c4c3o2)cc1 ; c1ccc(-c2nc3ccc4ccc5ccc(-c6ccc7c(-c8ccccc8)nc(-c8ccc9ccccc9c8)nc7c6)cc5c4c3o2)cc1]

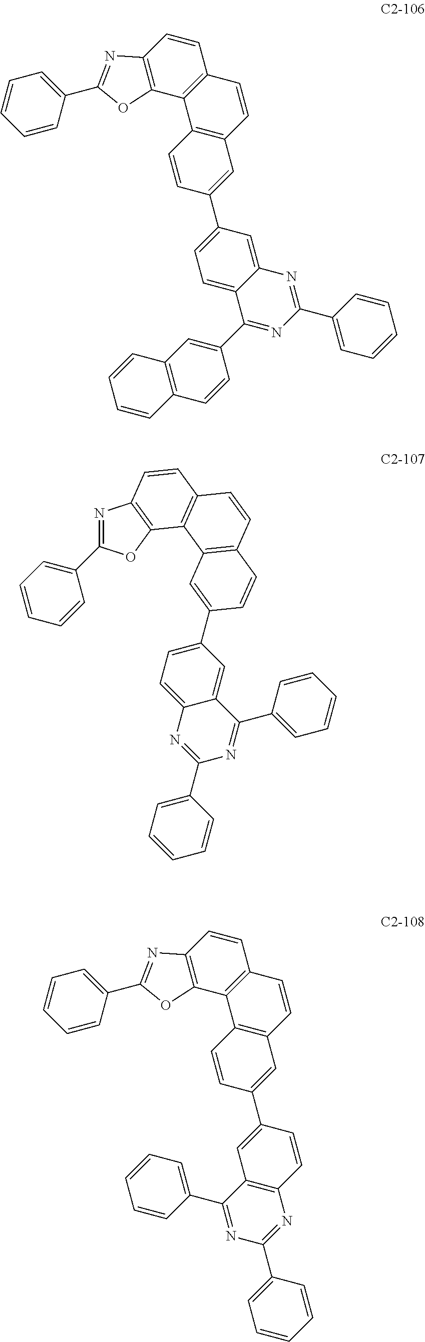 [SMILES: c1ccc(-c2nc(-c3ccc4ccccc4c3)c3ccc(-c4ccc5c(ccc6ccc7nc(-c8ccccc8)oc7c65)c4)cc3n2)cc1 ; c1ccc(-c2nc(-c3ccccc3)c3cc(-c4ccc5ccc6ccc7nc(-c8ccccc8)oc7c6c5c4)ccc3n2)cc1 ; c1ccc(-c2nc(-c3ccccc3)c3cc(-c4ccc5c(ccc6ccc7nc(-c8ccccc8)oc7c65)c4)ccc3n2)cc1]

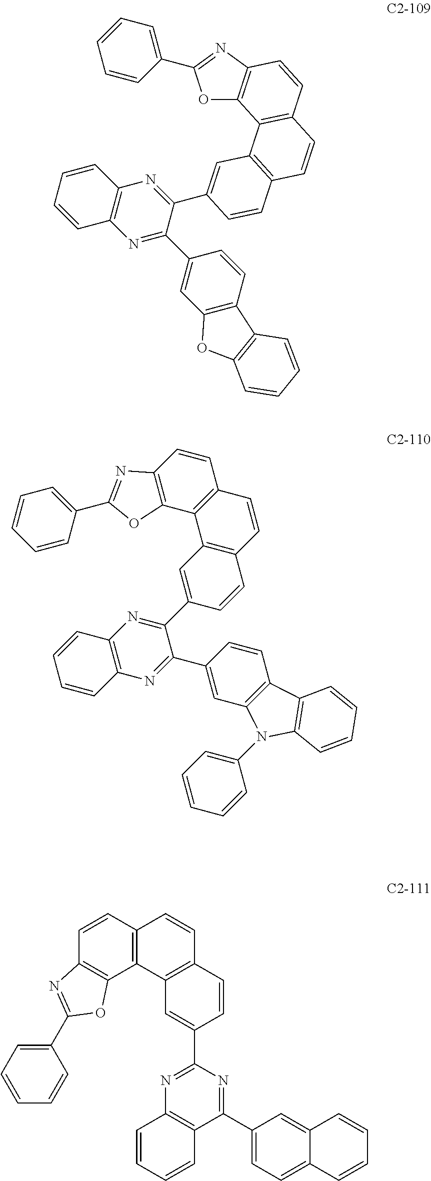 [SMILES: c1ccc(-c2nc3ccc4ccc5ccc(-c6nc7ccccc7nc6-c6ccc7c(c6)oc6ccccc67)cc5c4c3o2)cc1 ; c1ccc(-c2nc3ccc4ccc5ccc(-c6nc7ccccc7nc6-c6ccc7c8ccccc8n(-c8ccccc8)c7c6)cc5c4c3o2)cc1 ; c1ccc(-c2nc3ccc4ccc5ccc(-c6nc(-c7ccc8ccccc8c7)c7ccccc7n6)cc5c4c3o2)cc1]

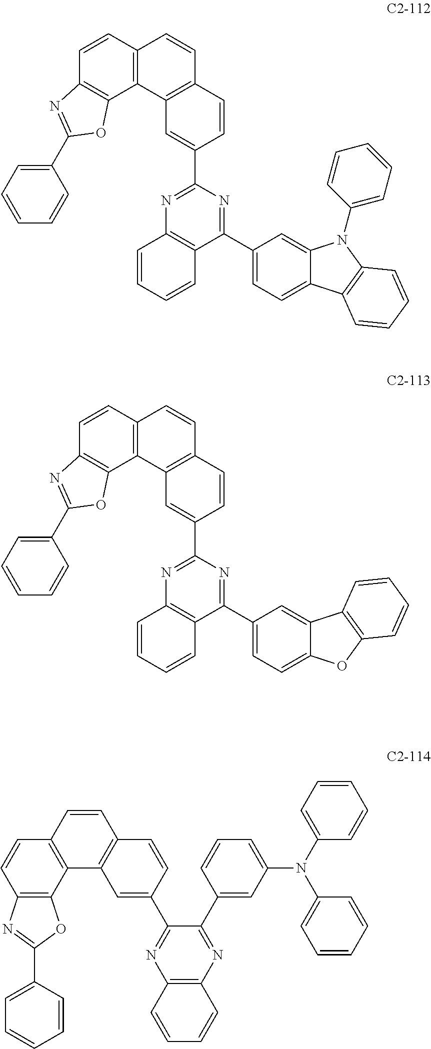 [SMILES: c1ccc(-c2nc3ccc4ccc5ccc(-c6nc(-c7ccc8c9ccccc9n(-c9ccccc9)c8c7)c7ccccc7n6)cc5c4c3o2)cc1 ; c1ccc(-c2nc3ccc4ccc5ccc(-c6nc(-c7ccc8oc9ccccc9c8c7)c7ccccc7n6)cc5c4c3o2)cc1 ; c1ccc(-c2nc3ccc4ccc5ccc(-c6nc7ccccc7nc6-c6cccc(N(c7ccccc7)c7ccccc7)c6)cc5c4c3o2)cc1]

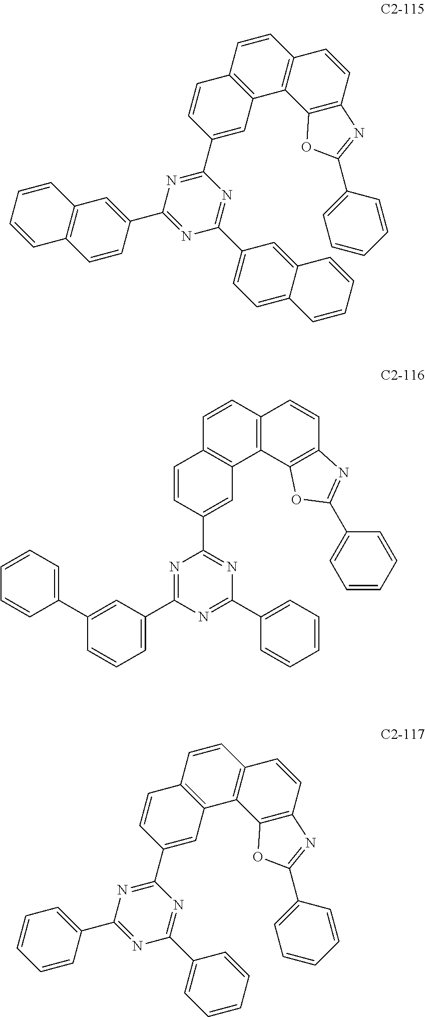 [SMILES: c1ccc(-c2nc3ccc4ccc5ccc(-c6nc(-c7ccc8ccccc8c7)nc(-c7ccc8ccccc8c7)n6)cc5c4c3o2)cc1 ; c1ccc(-c2cccc(-c3nc(-c4ccccc4)nc(-c4ccc5ccc6ccc7nc(-c8ccccc8)oc7c6c5c4)n3)c2)cc1 ; c1ccc(-c2nc(-c3ccccc3)nc(-c3ccc4ccc5ccc6nc(-c7ccccc7)oc6c5c4c3)n2)cc1]

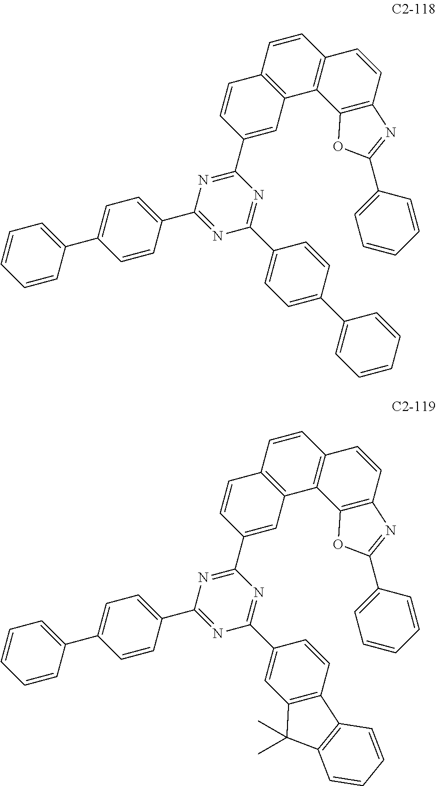 [SMILES: c1ccc(-c2ccc(-c3nc(-c4ccc(-c5ccccc5)cc4)nc(-c4ccc5ccc6ccc7nc(-c8ccccc8)oc7c6c5c4)n3)cc2)cc1 ; CC1(C)c2ccccc2-c2ccc(-c3nc(-c4ccc(-c5ccccc5)cc4)nc(-c4ccc5ccc6ccc7nc(-c8ccccc8)oc7c6c5c4)n3)cc21]

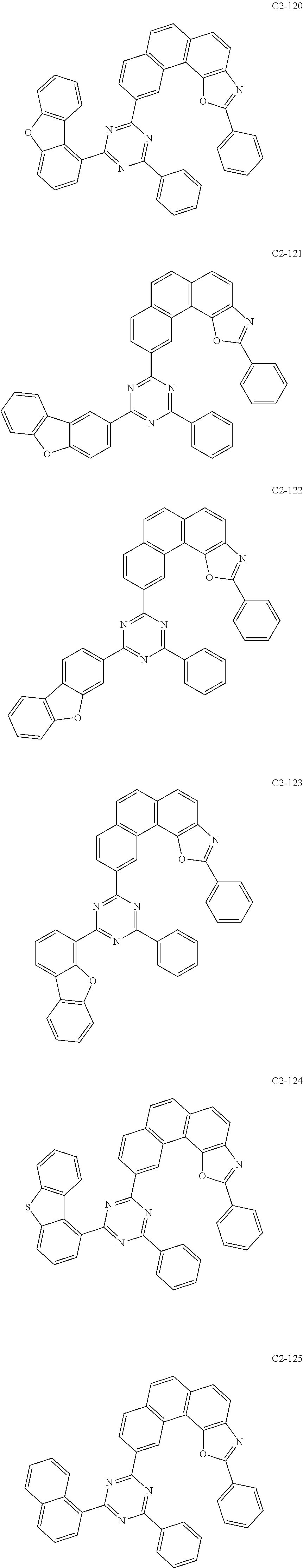 [SMILES: c1ccc(-c2nc(-c3ccc4ccc5ccc6nc(-c7ccccc7)oc6c5c4c3)nc(-c3cccc4oc5ccccc5c34)n2)cc1 ; c1ccc(-c2nc(-c3ccc4oc5ccccc5c4c3)nc(-c3ccc4ccc5ccc6nc(-c7ccccc7)oc6c5c4c3)n2)cc1 ; c1ccc(-c2nc(-c3ccc4c(c3)oc3ccccc34)nc(-c3ccc4ccc5ccc6nc(-c7ccccc7)oc6c5c4c3)n2)cc1 ; c1ccc(-c2nc(-c3ccc4ccc5ccc6nc(-c7ccccc7)oc6c5c4c3)nc(-c3cccc4c3oc3ccccc34)n2)cc1 ; c1ccc(-c2nc(-c3ccc4ccc5ccc6nc(-c7ccccc7)oc6c5c4c3)nc(-c3cccc4sc5ccccc5c34)n2)cc1 ; c1ccc(-c2nc(-c3ccc4ccc5ccc6nc(-c7ccccc7)oc6c5c4c3)nc(-c3cccc4ccccc34)n2)cc1]

The combination of at least one of compounds C1-1 to C1-94 and at least one of compounds C2-1 to C2-125 may be used in an organic electroluminescent device.

The compound represented by formula 1 according to the present disclosure may be prepared by a synthetic method known to one skilled in the art. For example, the compound represented by formula 1 can be prepared by referring to the following reaction scheme 1, and Korean Patent Appl. Laid-Open Nos. 2015-0135109 A (published on Dec. 2, 2015), 2015-0032447 A (published on Mar. 26, 2015), 2016-0099471 A (published on Aug. 22, 2016), 2018-0012709 A (published on Feb. 6, 2018), 2012-0132815 A (published on Dec. 10, 2012), 2015-0077513 A (published on Jul. 8, 2015), and 2017-0129599 A (published on Nov. 27, 2017), and Korean Patent No. 1478990 B (published on Dec. 29, 2014), but is not limited thereto.

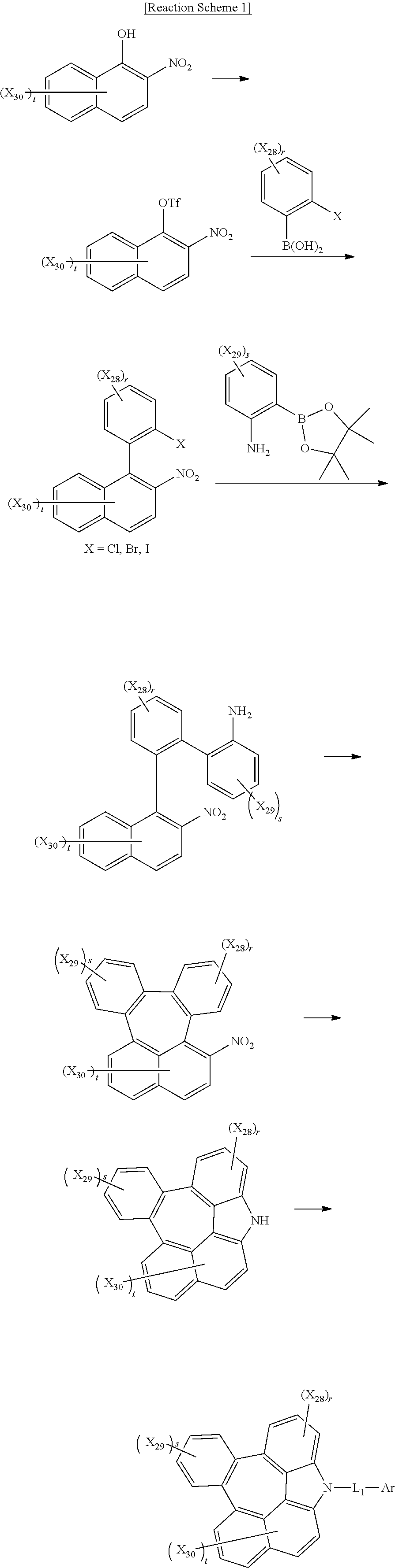

In reaction scheme 1, Ar, L₁, X₂₈ to X₃₀, r, s, and t are as defined in formula 1-9 above.

The compound represented by formula 2 according to the present disclosure may be prepared by a synthetic method known to one skilled in the art. For example, the compound represented by formula 2 can be prepared by referring to Korean Patent Appl. Laid-Open No. 2017-0022865 A (published on Mar. 2, 2017), but is not limited thereto.

The organic electroluminescent device according to the present disclosure comprises an anode, a cathode, and at least one organic layer between the anode and the cathode. The organic layer may comprise a plurality of organic electroluminescent materials in which the compound represented by formula 1 is included as a first organic electroluminescent material, and the compound represented by formula 2 is included as a second organic electroluminescent material. According to one embodiment of the present disclosure, the organic electroluminescent device comprises an anode, a cathode, and at least one light-emitting layer between the anode and the cathode, and at least one layer of the at least one light-emitting layer may comprise the compound represented by formula 1 and the compound represented by formula 2.

The light-emitting layer comprises a host and a dopant. The host comprises a plurality of host materials. The compound represented by formula 1 may be comprised as a first host compound in a plurality of host materials, and the compound represented by formula 2 may be comprised as a second host compound in a plurality of host materials. The weight ratio of the first host compound to the second host compound is in the range of about 1:99 to about 99:1, preferably about 10:90 to about 90:10, more preferably about 30:70 to about 70:30, even more preferably about 40:60 to 60:40, and still more preferably about 50:50.

The light-emitting layer is a layer from which light is emitted, and can be a single layer or a multi-layer in which two or more layers are stacked. In the plurality of host materials according to the present disclosure, the first and second host materials may both be comprised in one layer, or may be respectively comprised in different light-emitting layers. According to one embodiment of the present disclosure, the doping concentration of the dopant compound with respect to the host compound in the light-emitting layer is less than 20 wt %.

The organic electroluminescent device of the present disclosure may further comprise at least one layer selected from a hole injection layer, a hole transport layer, a hole auxiliary layer, a light-emitting auxiliary layer, an electron transport layer, an electron injection layer, an interlayer, an electron buffer layer, a hole blocking layer, and an electron blocking layer. According to one embodiment of the present disclosure, the organic electroluminescent device may further comprise amine-based compounds in addition to a plurality of host materials of the present disclosure as at least one of a hole injection material, a hole transport material, a hole auxiliary material, a light-emitting material, a light-emitting auxiliary material and an electron blocking material. Also, according to one embodiment of the present disclosure, the organic electroluminescent device may further comprise azine-based compounds in addition to a plurality of host materials of the present disclosure as at least one of an electron transport material, an electron injection material, an electron buffer material and a hole blocking material.

The dopant comprised in the organic electroluminescent device of the present disclosure may be at least one phosphorescent or fluorescent dopant, preferably a phosphorescent dopant. The phosphorescent dopant material is not particulary limited, but may be preferably selected from the metallated complex compounds of iridium (Ir), osmium (Os), copper (Cu), and platinum (Pt), more preferably selected from ortho-metallated complex compounds of iridium (Ir), osmium (Os), copper (Cu), and platinum (Pt), and even more preferably ortho-metallated iridium complex compounds.

The dopant compound comprised in the organic electroluminescent device of the present disclosure, may comprise the compound represented by the following formula 101, but is not limited thereto.

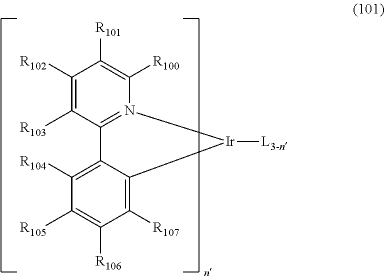

In formula 101, L is selected from the following structures 1 and 2:

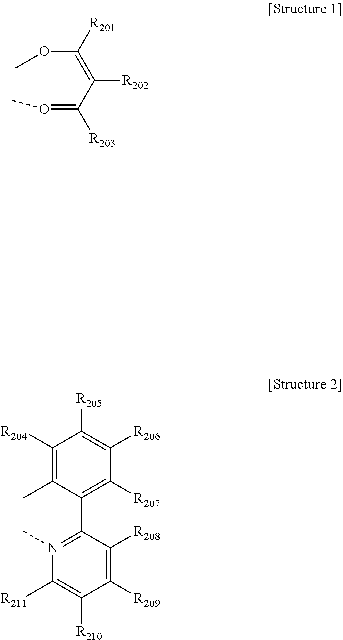

R₁₀₀ to R₁₀₃, each independently, represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30)aryl, a cyano, a substituted or unsubstituted (3- to 30-membered)heteroaryl, or a substituted or unsubstituted (C1-C30)alkoxy; or may be linked to adjacent R₁₀₀ to R₁₀₃, to form a ring(s) together with pyridine, e.g., a substituted or unsubstituted quinoline, a substituted or unsubstituted benzofuropyridine, a substituted or unsubstituted benzothienopyridine, a substituted or unsubstituted indenopyridine, a substituted or unsubstituted benzofuroquinoline, a substituted or unsubstituted benzothienoquinoline, or a substituted or unsubstituted indenoquinoline ring;

R₁₀₄ to R₁₀₇, each independently, represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30)aryl, a substituted or unsubstituted (3- to 30-membered)heteroaryl, a cyano, or a substituted or unsubstituted (C1-C30)alkoxy; or may be linked to adjacent R₁₀₄ to R₁₀₇ to form a ring(s) together with benzene, e.g., a substituted or unsubstituted naphthalene, a substituted or unsubstituted fluorene, a substituted or unsubstituted dibenzothiophene, a substituted or unsubstituted dibenzofuran, a substituted or unsubstituted indenopyridine, a substituted or unsubstituted benzofuropyridine, or a substituted or unsubstituted benzothienopyridine ring;

R₂₀₁ to R₂₁₁, each independently, represent hydrogen, deuterium, a halogen, a (C1-C30)alkyl unsubstituted or substituted with deuterium and/or a halogen(s), a substituted or unsubstituted (C3-C30)cycloalkyl, or a substituted or unsubstituted (C6-C30)aryl; or may be linked to adjacent R₂₀₁ to R₂₁₁ to form a ring; and

n′ represents an integer of 1 to 3.

The specific examples of the dopant compound are as follows, but are not limited thereto.

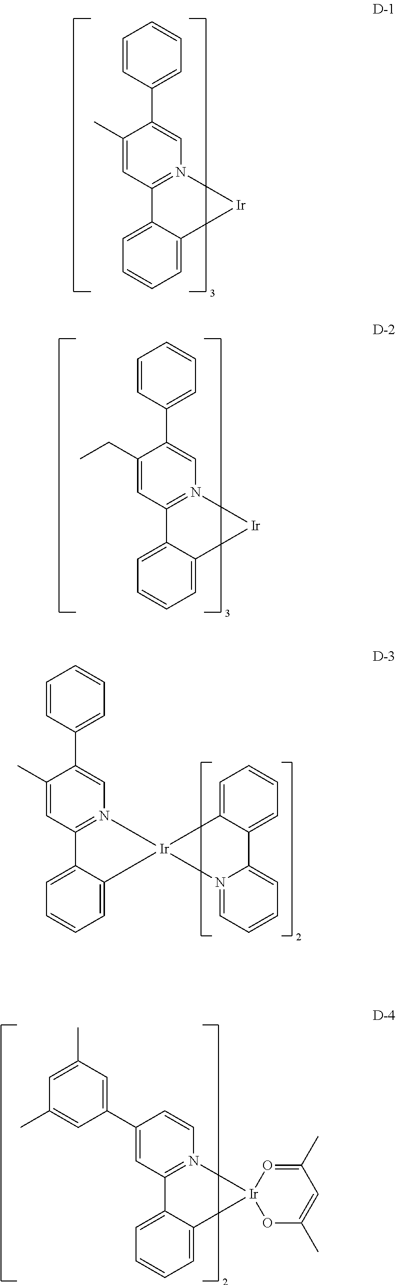

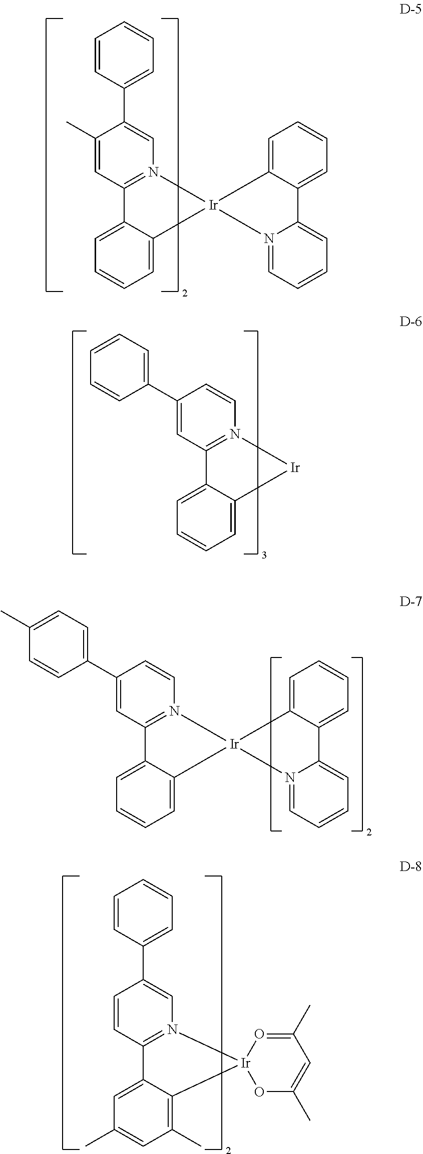

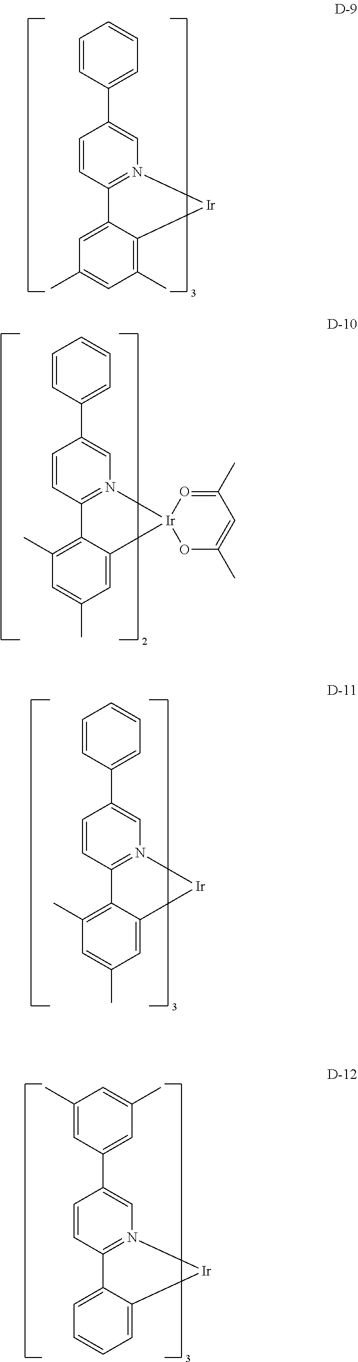

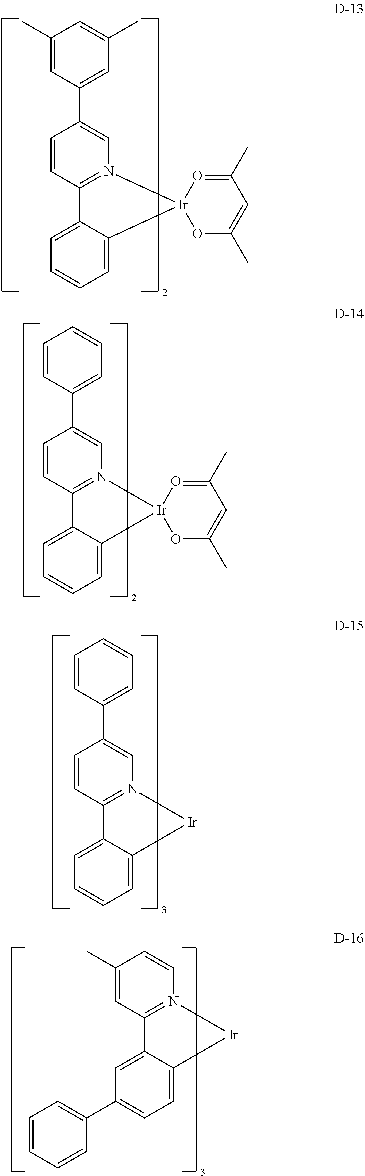

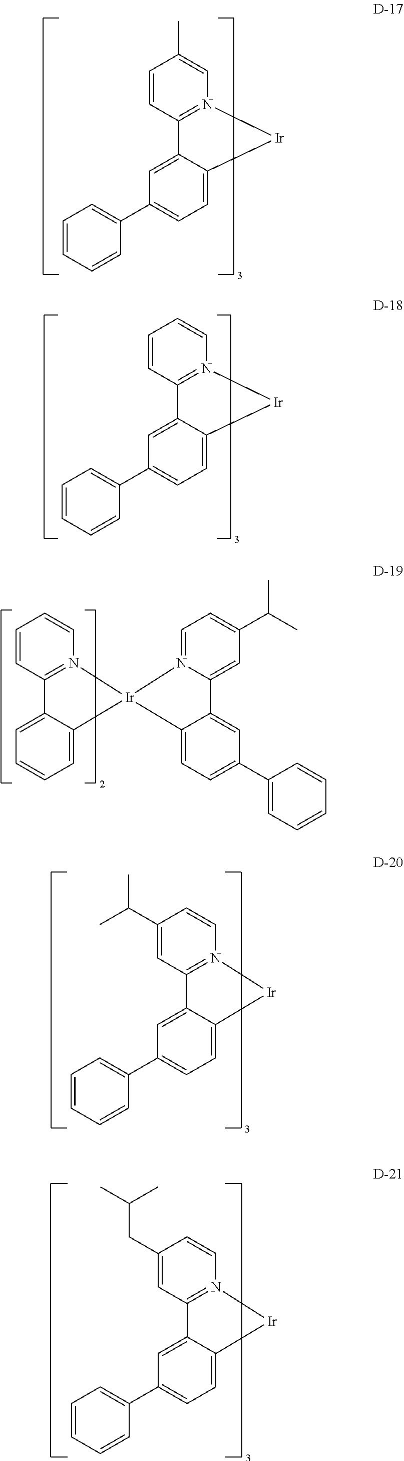

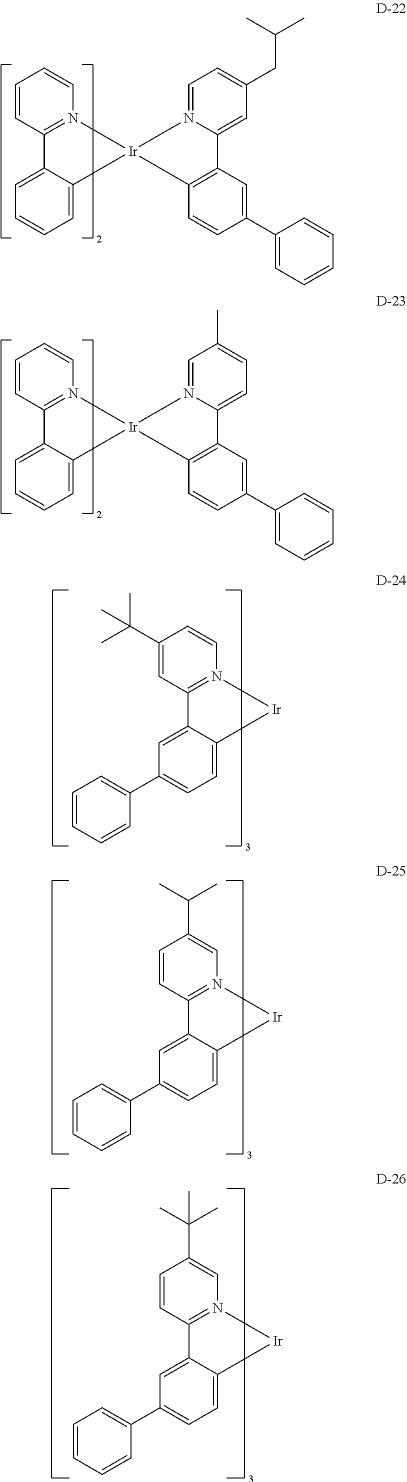

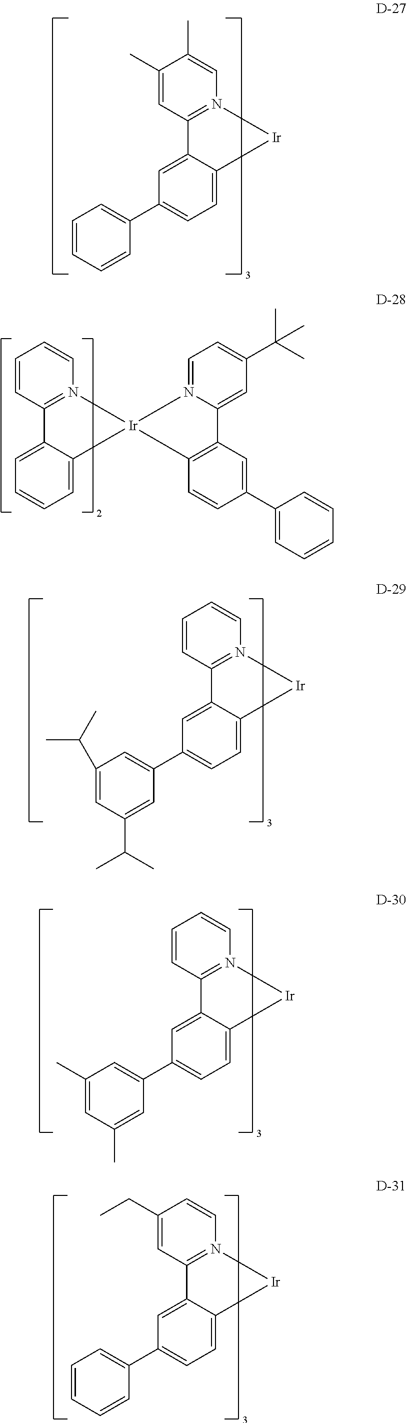

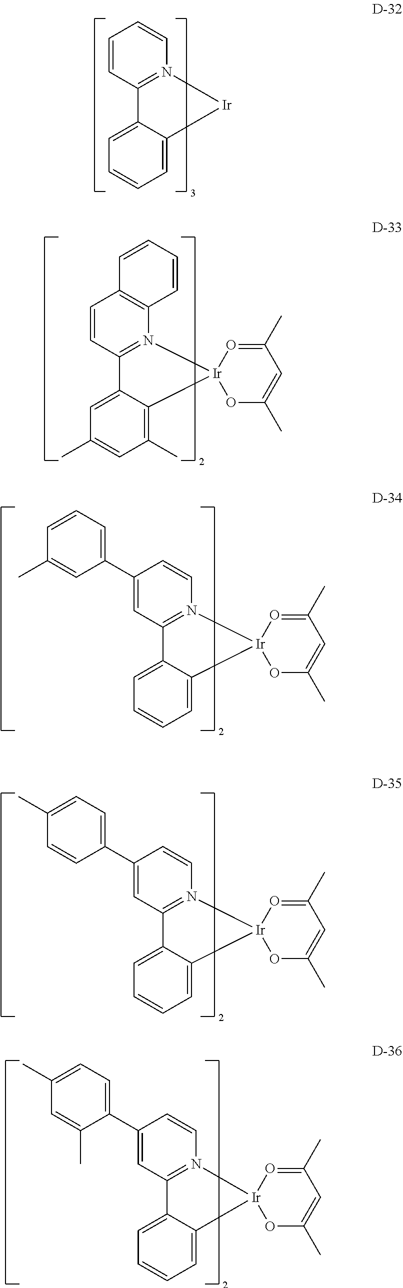

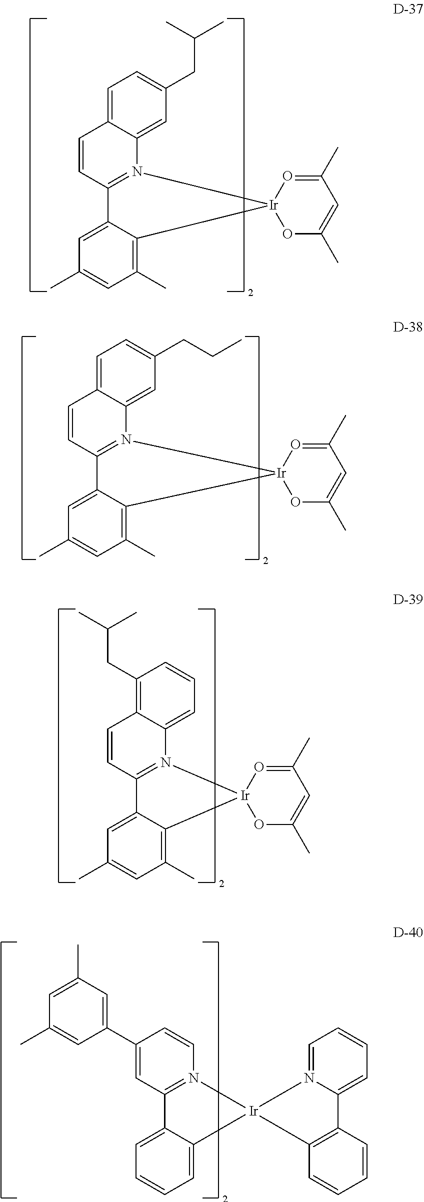

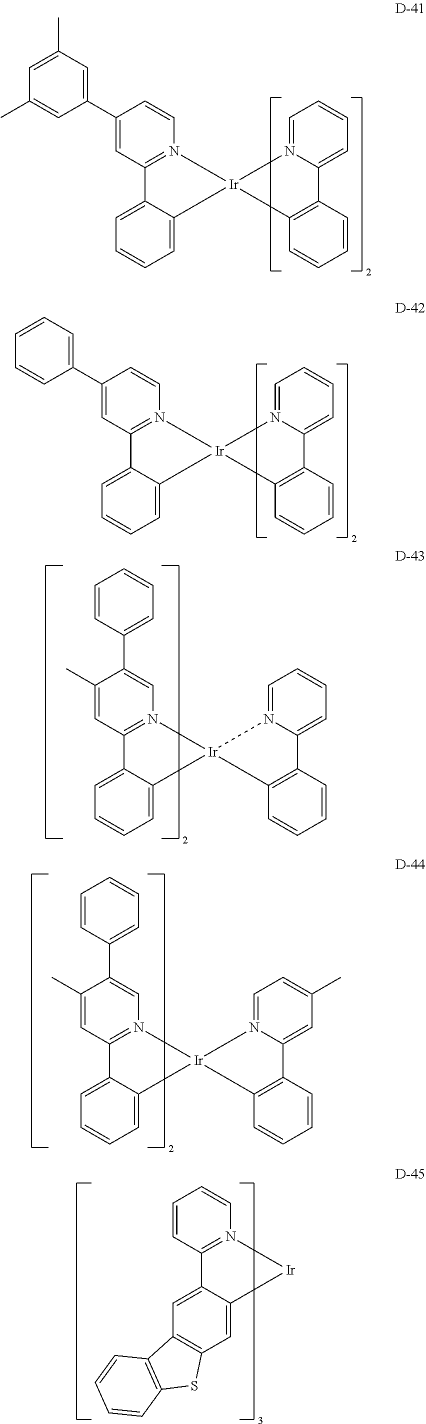

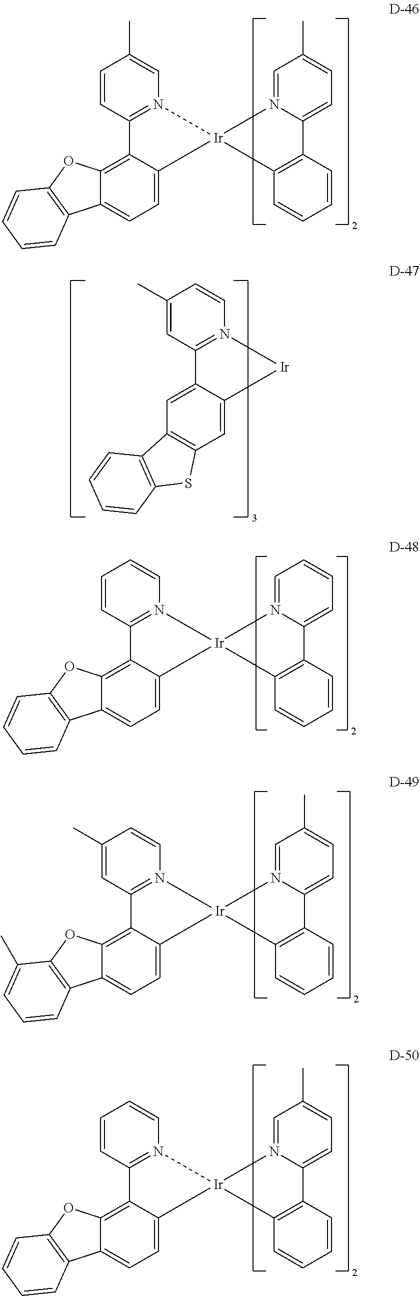

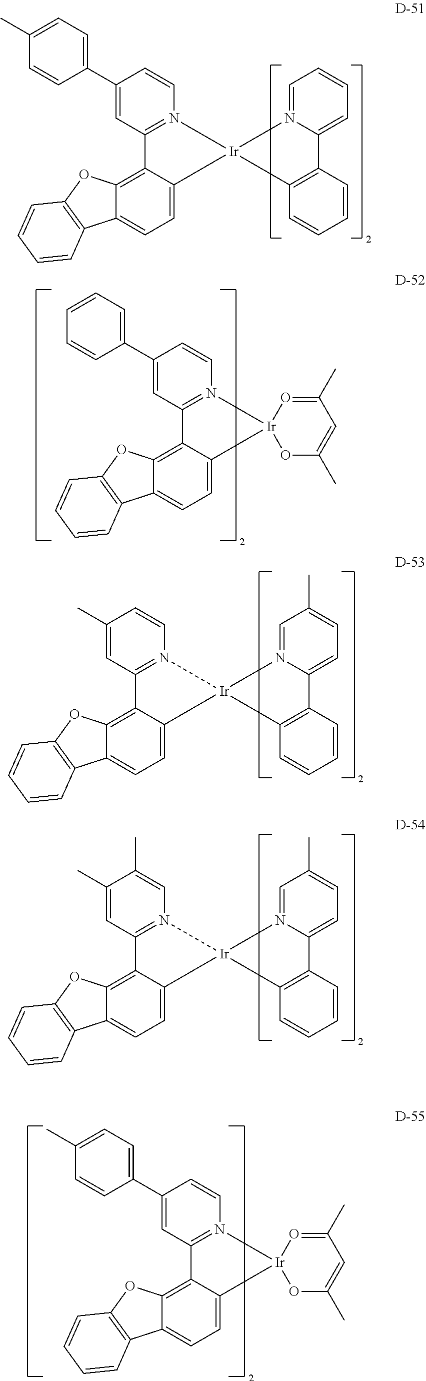

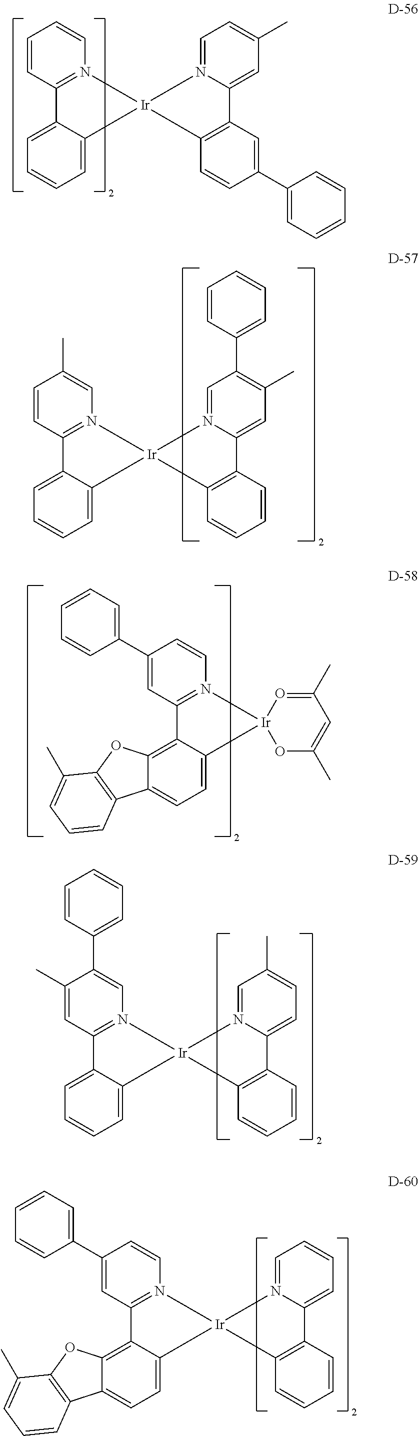

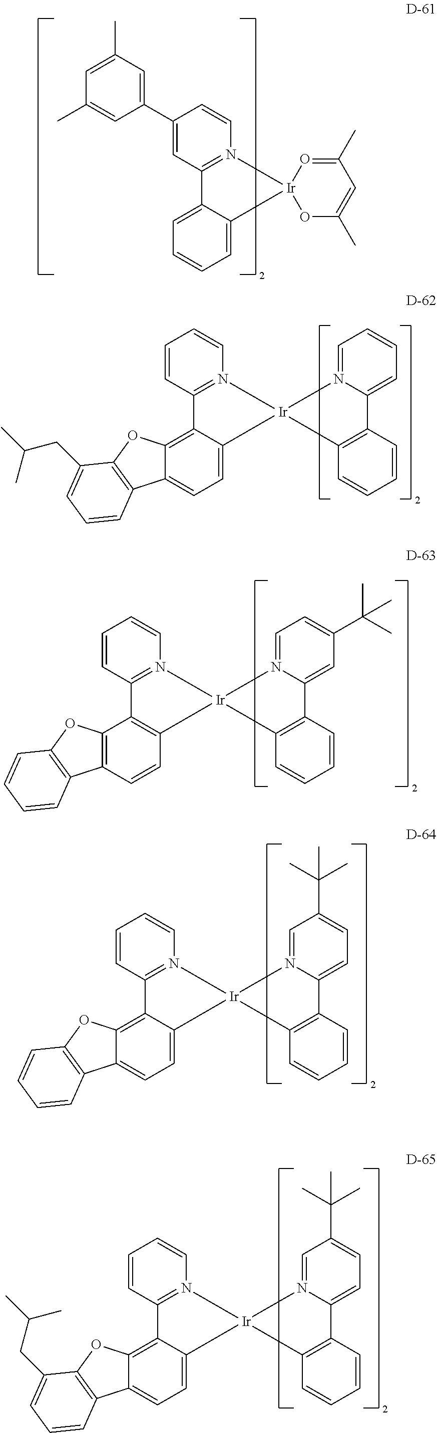

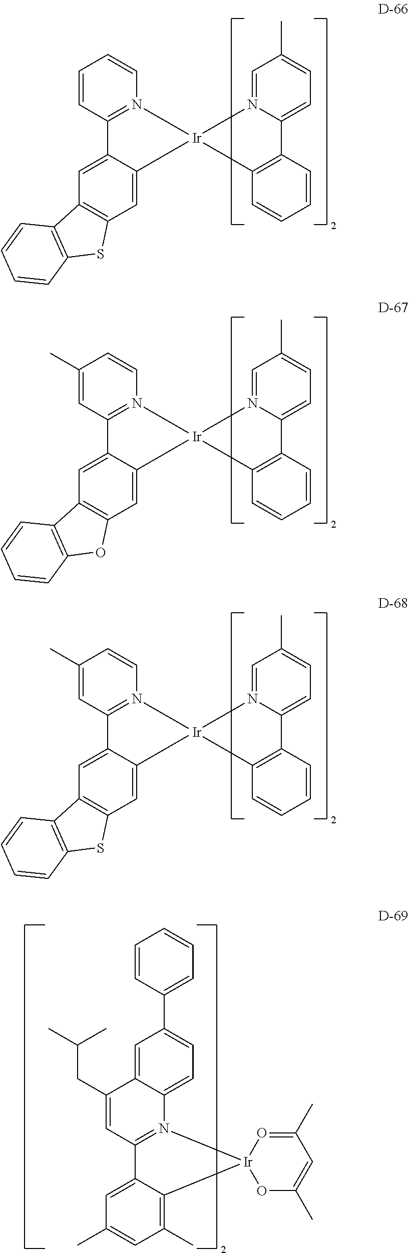

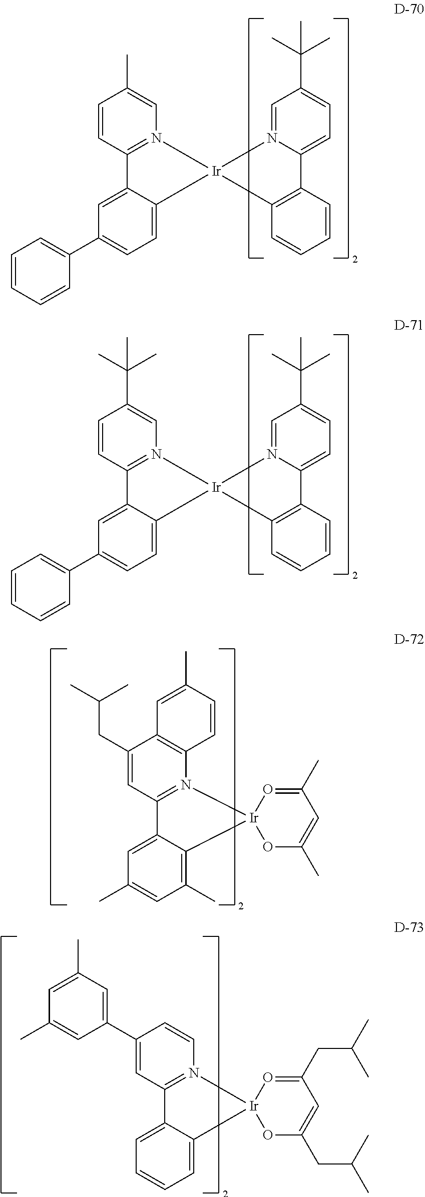

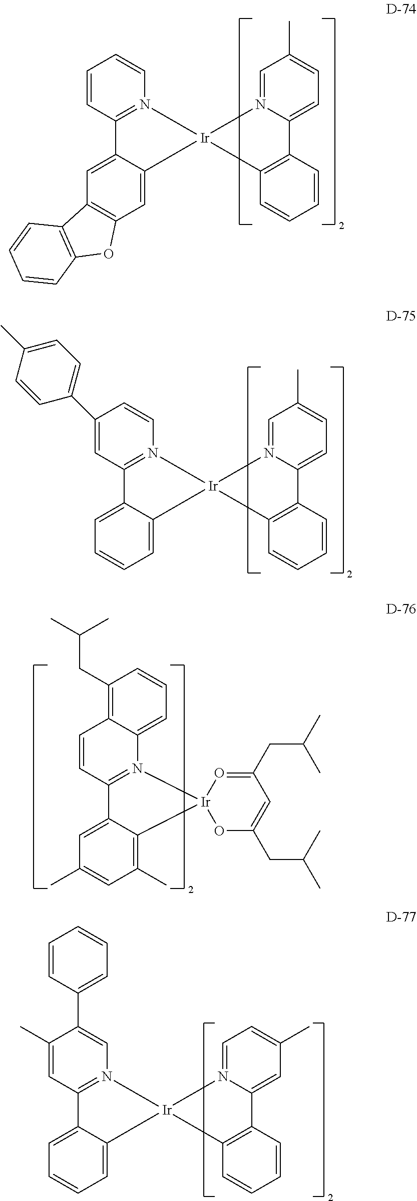

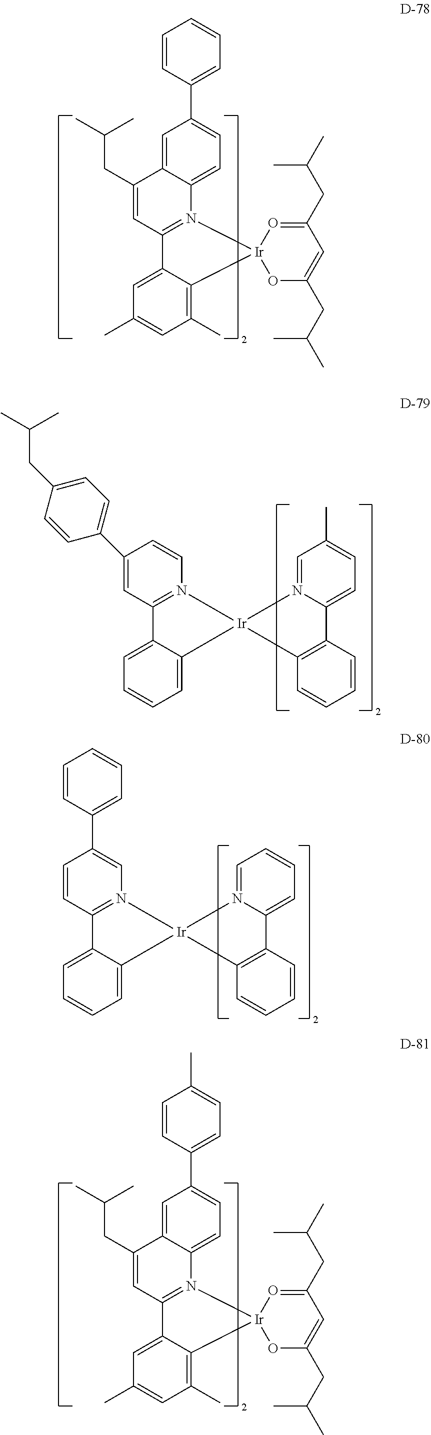

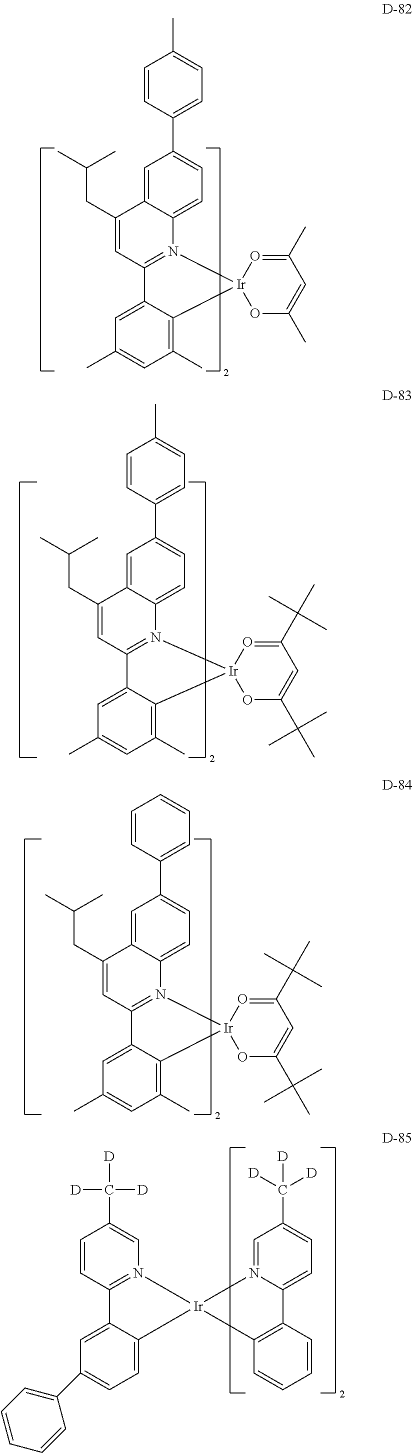

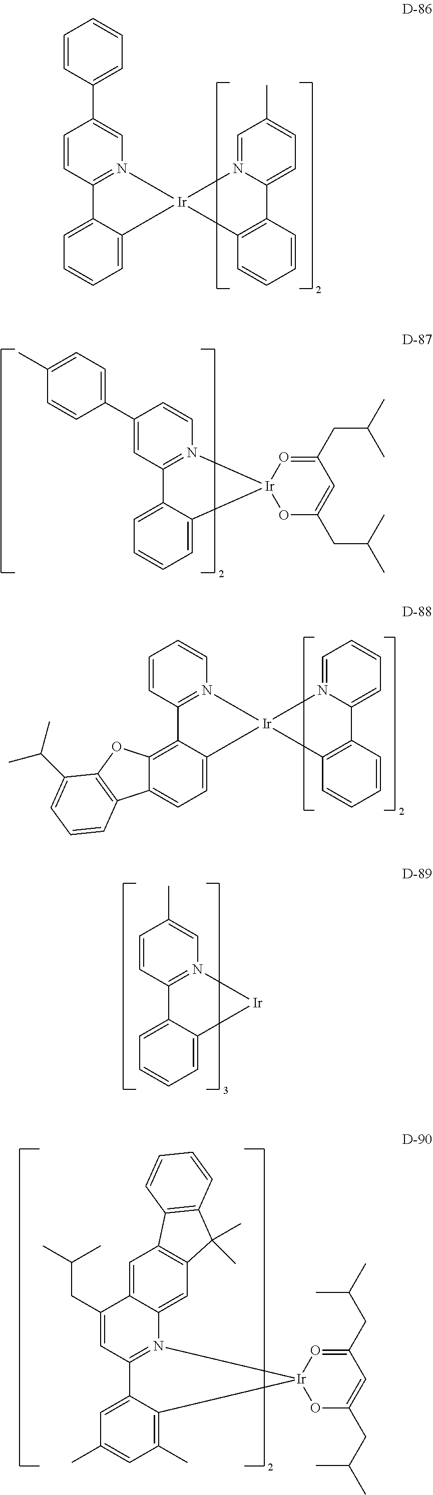

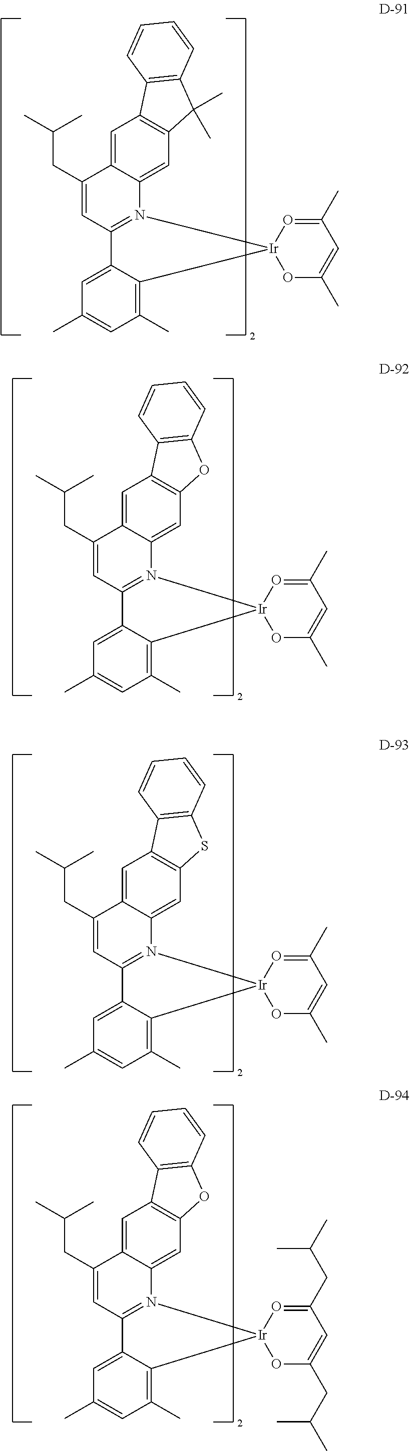

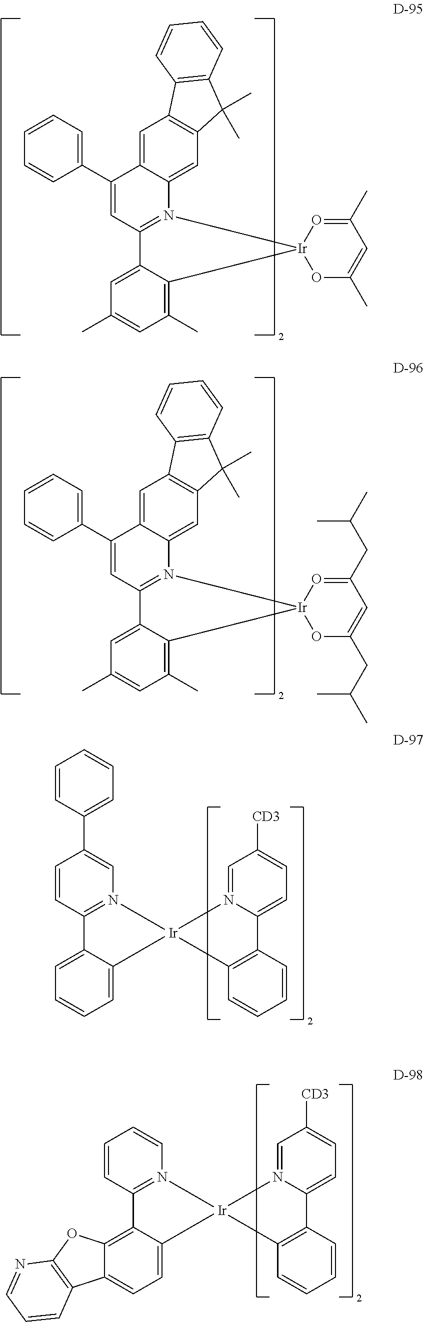

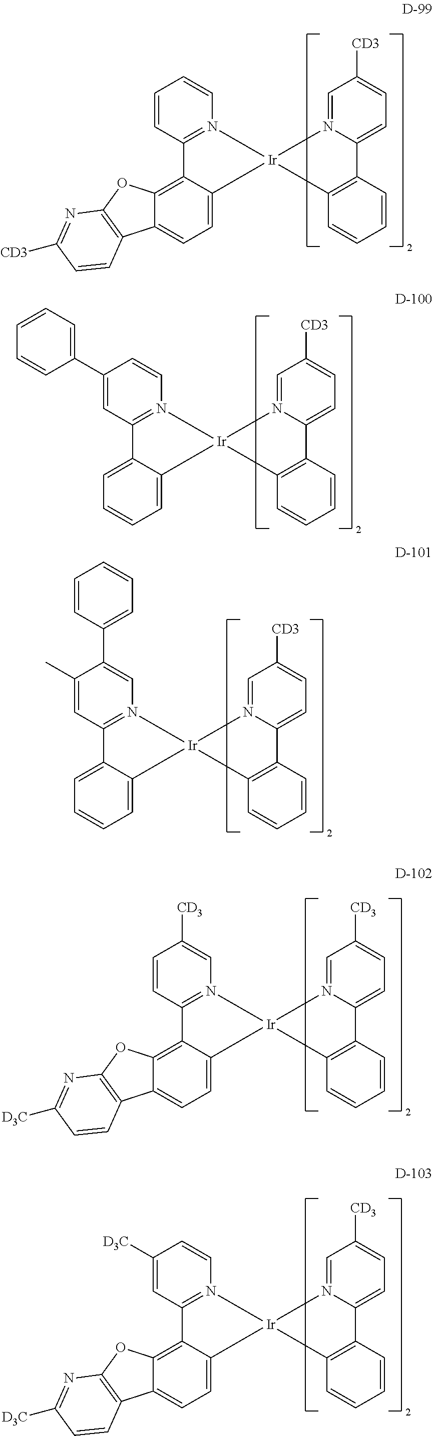

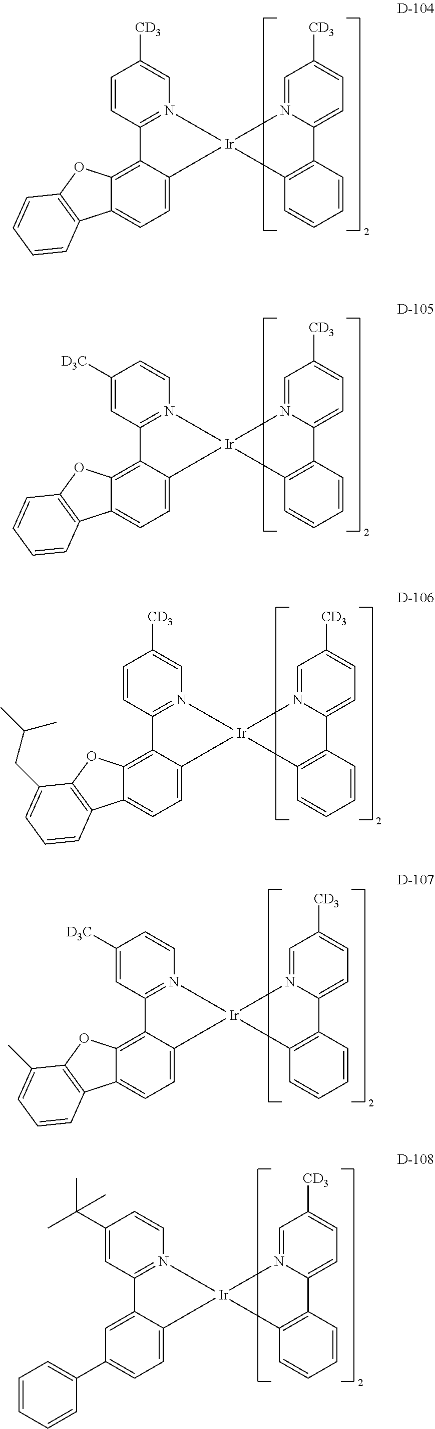

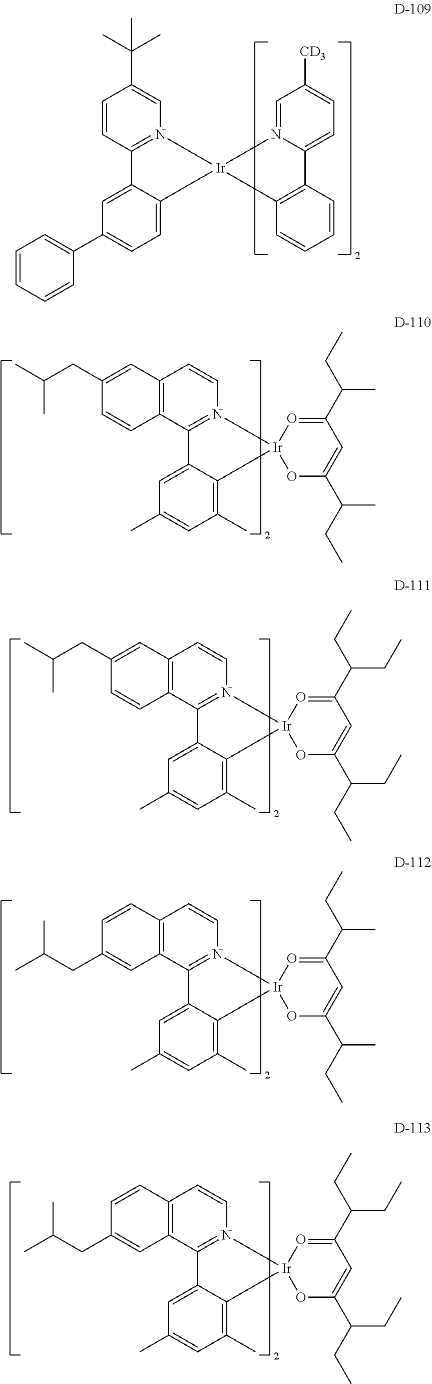

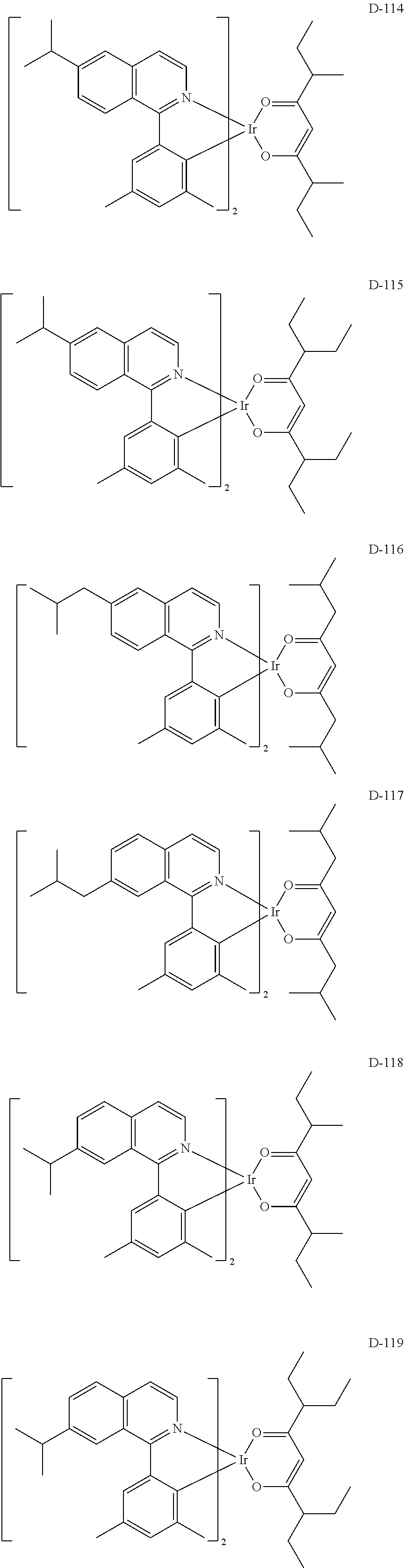

In order to form each layer of the organic electroluminescent device of the present disclosure, dry film-forming methods such as vacuum evaporation, sputtering, plasma, and ion plating methods, or wet film-forming methods such as ink jet printing, nozzle printing, slot coating, spin coating, dip coating, and flow coating methods can be used.

When using a solvent in a wet film-forming method, a thin film can be formed by dissolving or diffusing materials forming each layer into any suitable solvent such as ethanol, chloroform, tetrahydrofuran, dioxane, etc. The solvent can be any solvent where the materials forming each layer can be dissolved or diffused, and where there are no problems in film-formation capability.

In addition, the compound represented by formula 1, and the compound represented by formula 2 may be film-formed in the above-listed methods, commonly by a co-evaporation process or a mixture-evaporation process. The co-evaporation is a mixed deposition method in which two or more materials are placed in a respective individual crucible source and an electric current is applied to both cells at the same time to evaporate the materials. The mixture-evaporation is a mixed deposition method in which two or more materials are mixed in one crucible source before evaporating them, and an electric current is applied to the cell to evaporate the materials.

The present disclosure may provide a display system by comprising a plurality of host materials. In addition, it is possible to produce a display system or a lighting system by using the organic electroluminent device of the present disclosure. Specifically, it is possible to produce a display system, e.g., a display system for smartphones, tablets, notebooks, PCs, TVs, or cars, or a lighting system, e.g., an outdoor or indoor lighting system, by using the plurality of host materials of the present disclosure.

Hereinafter, the luminous efficiency and the lifespan properties of an OLED according to the present disclosure will be explained. However, the following examples merely illustrate the properties of an OLED according to the present disclosure in detail, but the present disclosure is not limited to the following examples.

### Device Examples 1, 2, and 5 to 12: Producing an OLED by Co-Evaporating the First and Second Host Compounds According to the Present Disclosure

An OLED according to the present disclosure was produced as follows: A transparent electrode indium tin oxide (ITO) thin film (10 Ω/sq) on a glass substrate for an OLED (GEOMATEC CO., LTD., Japan) was subjected to an ultrasonic washing with trichloroethylene, acetone, ethanol and distilled water, sequentially, and then was stored in isopropanol. The ITO substrate was mounted on a substrate holder of a vacuum vapor deposition apparatus. Compound HI-1 was introduced into a cell of the vacuum vapor deposition apparatus, and then the pressure in the chamber of the apparatus was then controlled to 10⁻⁶ torr. Thereafter, an electric current was applied to the cell to evaporate the above-introduced material, thereby forming a first hole injection layer having a thickness of 80 nm on the ITO substrate. Next, compound HI-2 was introduced into another cell of the vacuum vapor deposition apparatus and was evaporated by applying an electric current to the cell, thereby forming a second hole injection layer having a thickness of 5 nm on the first hole injection layer. Compound HT-1 was then introduced into another cell of the vacuum vapor deposition apparatus and was evaporated by applying an electric current to the cell, thereby forming a first hole transport layer having a thickness of 10 nm on the second hole injection layer. Compound HT-2 was then introduced into another cell of the vacuum vapor deposition apparatus and was evaporated by applying an electric current to the cell, thereby forming a second hole transport layer having a thickness of 60 nm on the first hole transport layer. After forming the hole injection layers and the hole transport layers, a light-emitting layer was formed thereon as follows: The first host and the second host compounds shown in Table 1 were introduced into two cells of the vacuum vapor depositing apparatus, respectively, as hosts and compound D-39 was introduced into another cell as a dopant. The two host materials were evaporated at a rate of 1:1, and at the same time the dopent material was evaporated at different rates to be deposited in a doping amount of 3 wt % based on the total amount of the hosts and dopant to form a light-emitting layer having a thickness of 40 nm on the second hole transport layer. Next, compound ET-1 and compound EI-1 were evaporated at a rate of 1:1 in two other cells to deposit an electron transport layer having a thickness of 35 nm on the light-emitting layer. After depositing compound EI-1 as an electron injection layer having a thickness of 2 nm on the electron transport layer, an Al cathode having a thickness of 80 nm was deposited on the electron injection layer by another vacuum vapor deposition apparatus. Thus, an OLED was produced.

### Device Example 3: Producing an OLED by Mixture-Evaporating the First and Second Host Compounds According to the Present Disclosure

An OLED was produced in the same manner as in Device Example 2, except that the first host compound and the second host compound described in Table 1 were deposited in one cell rather than in two cells in a vacuum deposition apparatus.

### Device Example 4: Producing an OLED by Co-Evaporating the First and Second Host Compounds According to the Present Disclosure

An OLED was produced in the same manner as in Device Example 2, except that compound D-78 was used as a dopant instead of compound D-39.

Comparative Examples 1 and 2: Producing an OLED not According to the Present Disclosure

An OLED was produced in the same manner as in Device Example 1, except that only the second host compound described in Table 1 below was used instead of the two hosts.

The results of the the luminous efficiency at a luminance of 5,000 nits, and the time taken to reduce the initial luminance of 100% to a luminance of 97% at a constant current in a luminance of 5,000 nits (T97) of the OLEDs produced in the Device Examples and the Comparative Examples, are shown in the following Table 1.

TABLE 1

| | | | Luminous | |
|---|---|---|---|---|
| | First | Second | Efficiency | Lifespan |
| | Host | Host | [cd/A] | T97 [hr] |
| Device Example 1 | C1-82 | C2-1 | 24.2 | 128 |
| Device Example 2 | C1-43 | C2-1 | 26.5 | 122 |
| Device Example 3 | C1-43 | C2-1 | 27.0 | 150 |
| Device Example 4 | C1-43 | C2-1 | 28.6 | 180 |
| Device Example 5 | C1-17 | C2-2 | 25.4 | 154 |
| Device Example 6 | C1-43 | C2-120 | 31.1 | 304 |
| Device Example 7 | C1-92 | C2-120 | 29.5 | 285 |
| Device Example 8 | C1-43 | C2-116 | 28.0 | 332 |
| Device Example 9 | C1-43 | C2-115 | 24.3 | 406 |
| Device Example 10 | C1-43 | C2-123 | 27.2 | 76 |
| Device Example 11 | C1-43 | C2-125 | 28.2 | 180 |
| Device Example 12 | C1-44 | C2-125 | 27.5 | 88 |
| Comparative Example 1 | — | C2-1 | 20.6 | 8.7 |
| Comparative Example 2 | — | C2-2 | 18.0 | 9 |

From Table 1, it can be confirmed that an organic electroluminescent device comprising a specific combination of compounds according to the present disclosure as host materials has higher luminous efficiency and/or longer lifespan properties as compared with the conventional organic electroluminescent device.

The compounds used in the Device Examples and the Comparative Examples are provided in Table 2 below.

TABLE 2

| | |
|---|---|
| Hole Injection Layer/ Hole Transport Layer | 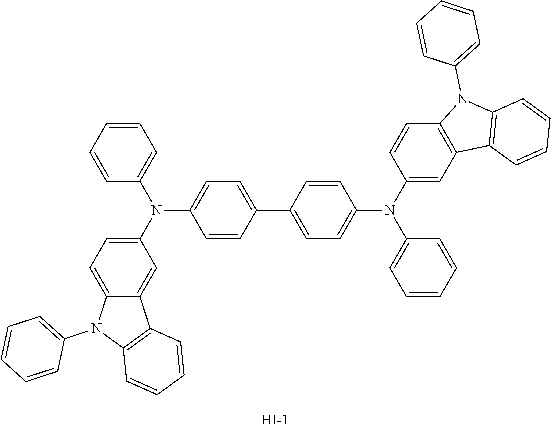 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; [I-]] |
| | 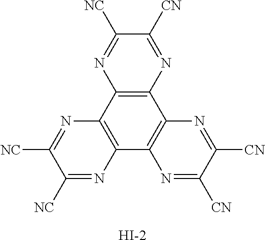 |
| | 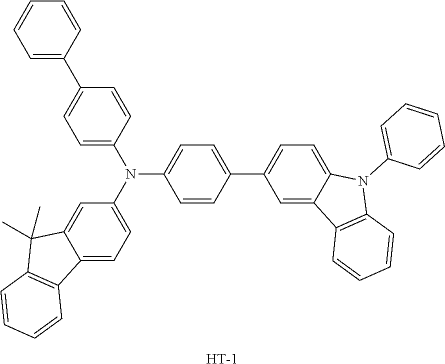 |
| | 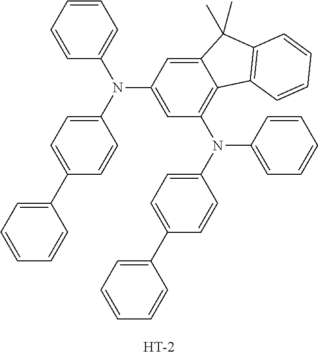 |
| Light-Emitting Layer | 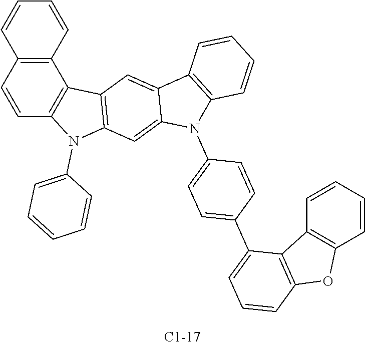 [SMILES: c1ccc(-n2c3cc4c(cc3c3c5ccccc5ccc32)c2ccccc2n4-c2ccc(-c3cccc4oc5ccccc5c34)cc2)cc1] |
| | 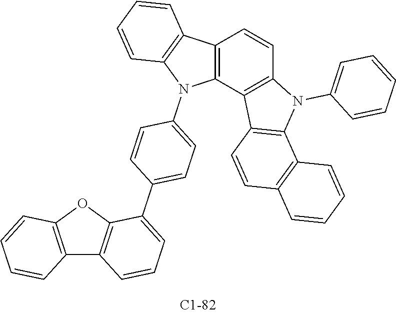 [SMILES: c1ccc(-n2c3ccc4c5ccccc5n(-c5ccc(-c6cccc7c6oc6ccccc67)cc5)c4c3c3ccc4ccccc4c32)cc1] |
| | 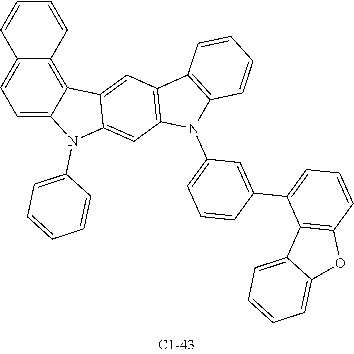 [SMILES: c1ccc(-n2c3cc4c(cc3c3c5ccccc5ccc32)c2ccccc2n4-c2cccc(-c3cccc4oc5ccccc5c34)c2)cc1] |
| | 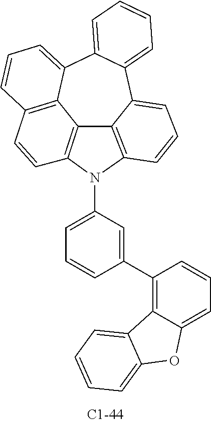 [SMILES: c1cc(-c2cccc3oc4ccccc4c23)cc(-n2c3cccc4c3c3c5c(cccc5ccc32)-c2ccccc2-4)c1] |
| | 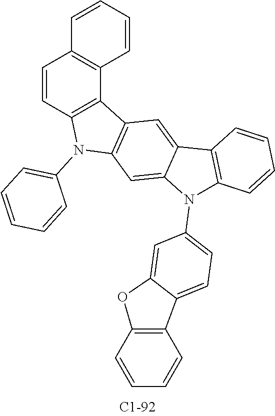 [SMILES: c1ccc(-n2c3cc4c(cc3c3c5ccccc5ccc32)c2ccccc2n4-c2ccc3c(c2)oc2ccccc23)cc1] |
| | 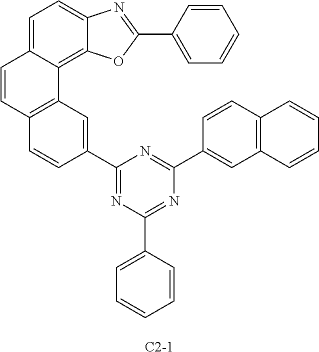 |
| | 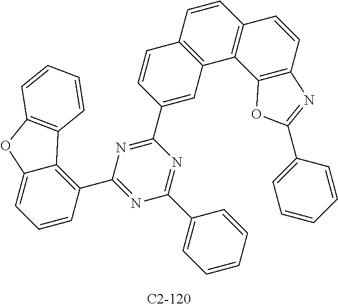 [SMILES: c1ccc(-c2nc(-c3ccc4ccc5ccc6nc(-c7ccccc7)oc6c5c4c3)nc(-c3cccc4oc5ccccc5c34)n2)cc1] |
| | 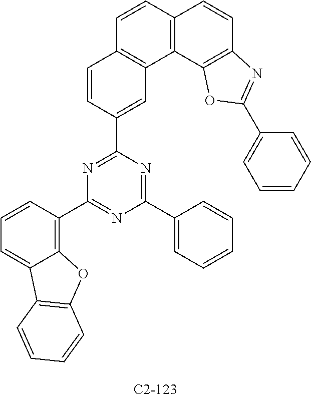 [SMILES: c1ccc(-c2nc(-c3ccc4ccc5ccc6nc(-c7ccccc7)oc6c5c4c3)nc(-c3cccc4c3oc3ccccc34)n2)cc1] |
| | 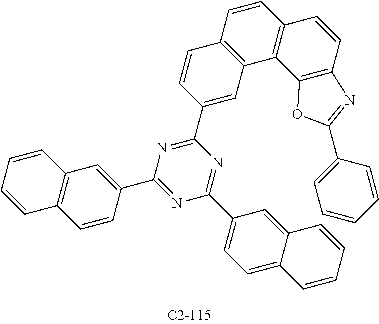 [SMILES: c1ccc(-c2nc3ccc4ccc5ccc(-c6nc(-c7ccc8ccccc8c7)nc(-c7ccc8ccccc8c7)n6)cc5c4c3o2)cc1] |
| | 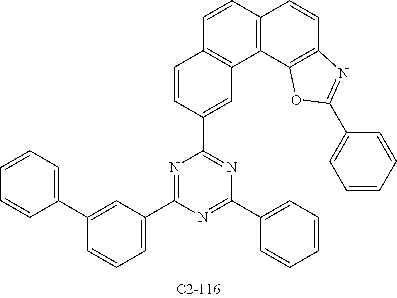 [SMILES: c1ccc(-c2cccc(-c3nc(-c4ccccc4)nc(-c4ccc5ccc6ccc7nc(-c8ccccc8)oc7c6c5c4)n3)c2)cc1] |
| | 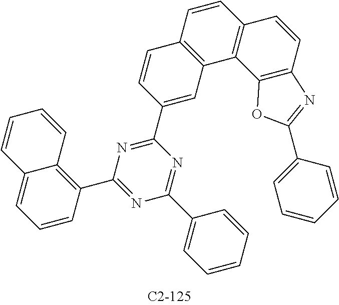 [SMILES: c1ccc(-c2nc(-c3ccc4ccc5ccc6nc(-c7ccccc7)oc6c5c4c3)nc(-c3cccc4ccccc34)n2)cc1] |
| | 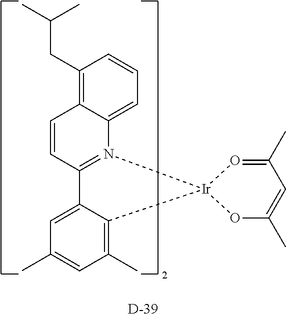 |
| | 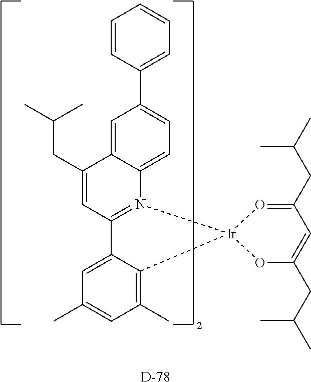 |
| Electron Transport Layer/ Electron Injection Layer | 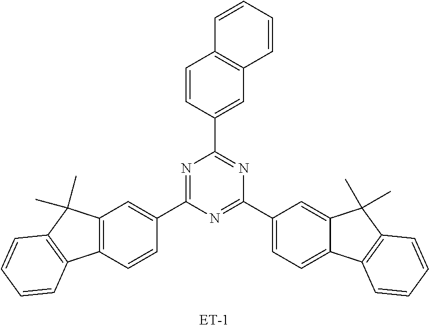 [SMILES: CC1(C)c2ccccc2-c2ccc(-c3nc(-c4ccc5c(c4)C(C)(C)c4ccccc4-5)nc(-c4ccc5ccccc5c4)n3)cc21] |
| | 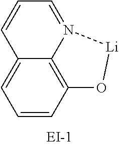 |

1. A plurality of host materials comprising a first host material and a second host material, wherein the first host material comprises a compound represented by the following formula 1: 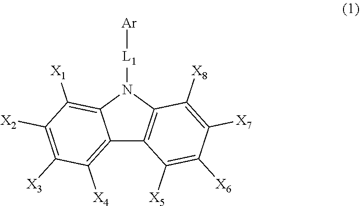
wherein,
Ar represents a substituted or unsubstituted (C6-C30)aryl, or a substituted or unsubstituted (3- to 30-membered)heteroaryl containing at least one of nitrogen, oxygen, and sulfur;
L₁ represents a single bond, a substituted or unsubstituted (C6-C30)arylene, or a substituted or unsubstituted (3- to 30-membered)heteroarylene;
X₁ to X₈, each independently, represent hydrogen, deuterium, a halogen, a cyano, a carboxyl, a nitro, a hydroxyl, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, a substituted or unsubstituted (3- to 30-membered)heteroaryl, —NR₅R₆, or —SiR₇R₈R₉; or adjacent ones of X₁ to X₈ may be linked to each other to form a ring(s); with the proviso that at least one pair of X₁ and X₂, X₂ and X₃, X₃ and X₄, X₄ and X₅, X₅ and X₆, X₆ and X₇, and X₇ and X₈ may be linked to each other to form a ring(s), in which the ring has 1 to 5 monocyclic rings;
R₅ to R₉, each independently, represent hydrogen, deuterium, a halogen, a cyano, a carboxyl, a nitro, a hydroxyl, a substituted or unsubstituted (C1-C30)alkyl, a substituted or unsubstituted (C3-C30)cycloalkyl, a substituted or unsubstituted (C3-C30)cycloalkenyl, a substituted or unsubstituted (3- to 7-membered)heterocycloalkyl, a substituted or unsubstituted (C6-C30)aryl, or a substituted or unsubstituted (3- to 30-membered)heteroaryl; or adjacent ones of R₅ to R₉ may be linked to each other to form a ring(s); and
the second host material comprises a compound represented by the following formula 2: 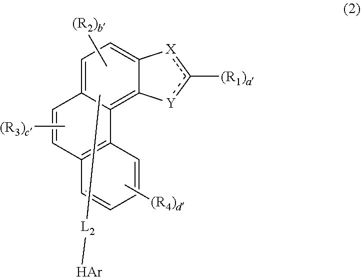
wherein,
X represents —N═, —NR₁₀—, —O—, or —S—;
Y represents —N═, —NR₁₁—, —O—, or —S—; with the proviso that when X represents —N═, Y represents —NR₁₁—, —O—, or —S—, and when X represents —NR₁₀—, Y represents —N═, —O—, or —S—;
HAr represents a substituted or unsubstituted (3- to 30-membered)heteroaryl containing a nitrogen atom(s);
L₂ represents a single bond, a substituted or unsubstituted (C6-C30)arylene, or a substituted or unsubstituted (3- to 30-membered)heteroarylene;
R₁ represents a substituted or unsubstituted (C6-C30)aryl, or a substituted or unsubstituted (3- to 30-membered)heteroaryl;
R₂ to R₄, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or adjacent ones of R₂ to R₄ may be linked to each other to form a ring(s);
R₁₀ and R₁₁, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino;
a′ represents an integer of 1; b′ and c′, each independently, represent an integer of 1 or 2; d′ represents an integer of 1 to 4; where if b′, c′, and d′, each independently, are an integer of 2 or more, each of R₂ to R₄ may be the same or different.

2. The plurality of host materials according to claim 1, wherein the substituents of the substituted aryl, the substituted arylene, the substituted heteroaryl, the substituted heteroarylene, the substituted alkyl, the substituted cycloalkyl, the substituted cycloalkenyl, the substituted heterocycloalkyl, the substituted alkoxy, the substituted trialkylsilyl, the substituted dialkylarylsilyl, the substituted alkyldiarylsilyl, the substituted triarylsilyl, the substituted mono- or di-alkylamino, the substituted mono- or di-arylamino, or the substituted alkylarylamino in Ar, L₁, X₁ to X₈, R₁ to R₁₁, HAr, and L₂, each independently, are at least one selected from the group consisting of deuterium; a halogen; a cyano; a carboxyl; a nitro; a hydroxyl; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 50-membered)heteroaryl unsubstituted or substituted with at least one of a (C1-C30)alkyl(s), a (C6-C30)aryl(s), and a di(C6-C30)arylamino(s); a (C6-C30)aryl unsubstituted or substituted with at least one of a cyano(s), a (C1-C30)alkyl(s), a (3- to 50-membered)heteroaryl(s), a di(C6-C30)arylamino(s), and a tri(C6-C30)arylsilyl(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; an amino; a mono- or di-(C1-C30)alkylamino; a mono- or di-(C6-C30)arylamino; a (C1-C30)alkyl(C6-C30)arylamino; a (C1-C30)alkylcarbonyl; a (C1-C30)alkoxycarbonyl; a (C6-C30)arylcarbonyl; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl.

3. The plurality of host materials according to claim 1, wherein the formula 1 is represented by any one of the following formulas 1-1 to 1-10: 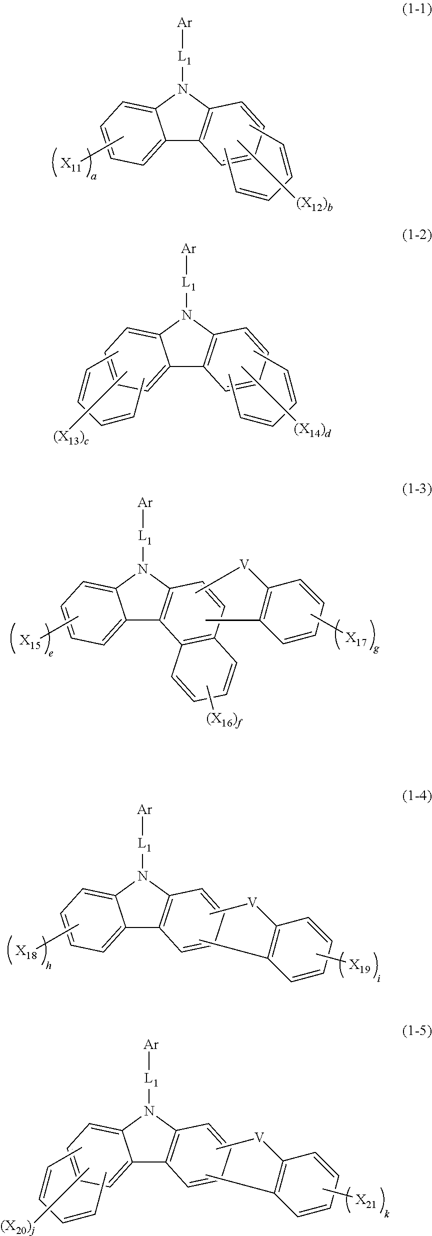 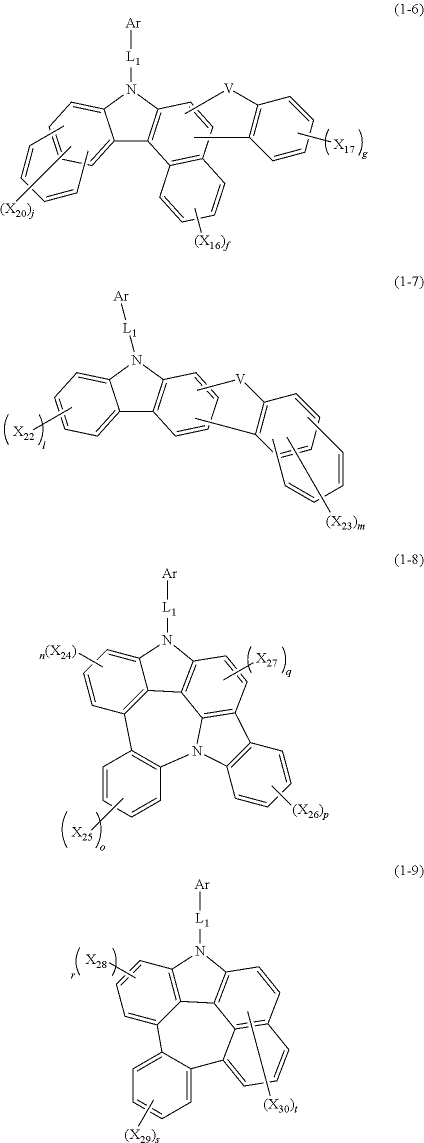 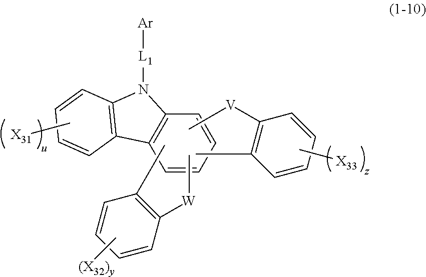
wherein,
Ar and L₁ are as defined in claim 1;
V and W, each independently, represent CR₁₂R₁₃, NR₁₄, O, or S;
R₁₂ to R₁₄, X₁₁ to X₂₃, and X₃₁ to X₃₃, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino;
X₂₄ to X₃₀, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or adjacent ones of X₂₄ to X₃₀ may be linked to each other to form a ring(s); and
a, e to i, k, l, o, p, s, u, y, and z, each independently, represent an integer of 1 to 4; b to d, j, and m, each independently, represent an integer of 1 to 6; n and r, each independently, represent an integer of 1 to 3; q represents an integer of 1 or 2; t represents an integer of 1 to 5; where a to u, y, and z, each independently, are an integer of 2 or more, each of X₁₁ to X₃₃ may be the same or different.

4. The plurality of host materials according to claim 1, wherein Ar represents a substituted or unsubstituted phenyl, a substituted or unsubstituted naphthyl, a substituted or unsubstituted biphenyl, a substituted or unsubstituted terphenyl, a substituted or unsubstituted carbazolyl, a substituted or unsubstituted dibenzothiophenyl, a substituted or unsubstituted benzothiophenyl, a substituted or unsubstituted dibenzofuranyl, a substituted or unsubstituted benzofuranyl, a substituted or unsubstituted naphthyridinyl, a substituted or unsubstituted fluorenyl, a substituted or unsubstituted benzofluorenyl, a substituted or unsubstituted triphenylenyl, a substituted or unsubstituted benzonaphthofuranyl, or a substituted or unsubstituted benzonaphthothiophenyl.

5. The plurality of host materials according to claim 1, wherein the formula 2 is represented by any one of the following formulas 2-1 and 2-2: 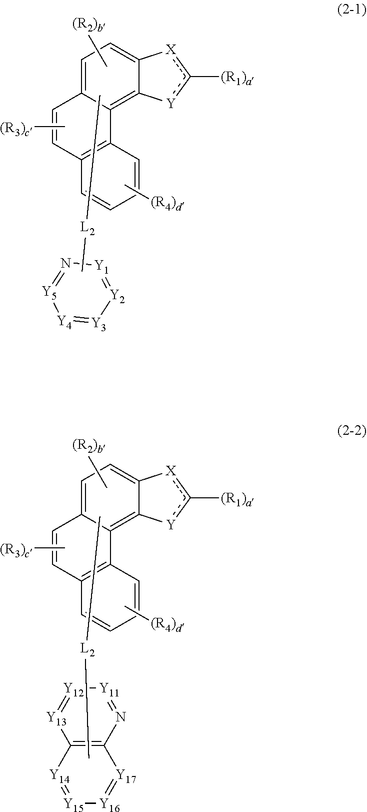
wherein,
X, Y, R₁ to R₄, L₂, and a′ to d′ are as defined in claim 1;
Y₁ to Y₅, and Y₁₁ to Y₁₇, each independently, represent N or CR₁₅; and
R₁₅, each independently, represents hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino; or adjacent ones of R₁₅'s may be linked to each other to form a ring(s).

6. The plurality of host materials according to claim 1, wherein HAr represents a substituted or unsubstituted triazinyl, a substituted or unsubstituted pyridyl, a substituted or unsubstituted pyrimidinyl, a substituted or unsubstituted quinazolinyl, a substituted or unsubstituted benzoquinazolinyl, a substituted or unsubstituted quinoxalinyl, a substituted or unsubstituted benzoquinoxalinyl, a substituted or unsubstituted quinolyl, a substituted or unsubstituted benzoquinolyl, a substituted or unsubstituted isoquinolyl, a substituted or unsubstituted benzoisoquinolyl, a substituted or unsubstituted triazolyl, a substituted or unsubstituted pyrazolyl, a substituted or unsubstituted naphthyridinyl, or a substituted or unsubstituted benzothienopyrimidinyl.

7. The plurality of host materials according to claim 1, wherein the compound represented by formula 1 is at least one selected from the group consisting of the following compounds:

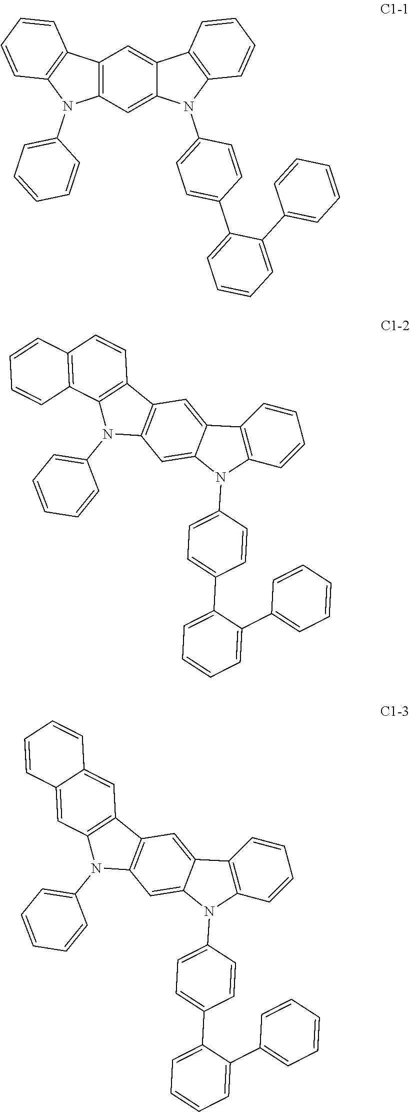 [SMILES: c1ccc(-c2ccccc2-c2ccc(-n3c4ccccc4c4cc5c6ccccc6n(-c6ccccc6)c5cc43)cc2)cc1 ; c1ccc(-c2ccccc2-c2ccc(-n3c4ccccc4c4cc5c6ccc7ccccc7c6n(-c6ccccc6)c5cc43)cc2)cc1 ; c1ccc(-c2ccccc2-c2ccc(-n3c4ccccc4c4cc5c6cc7ccccc7cc6n(-c6ccccc6)c5cc43)cc2)cc1]

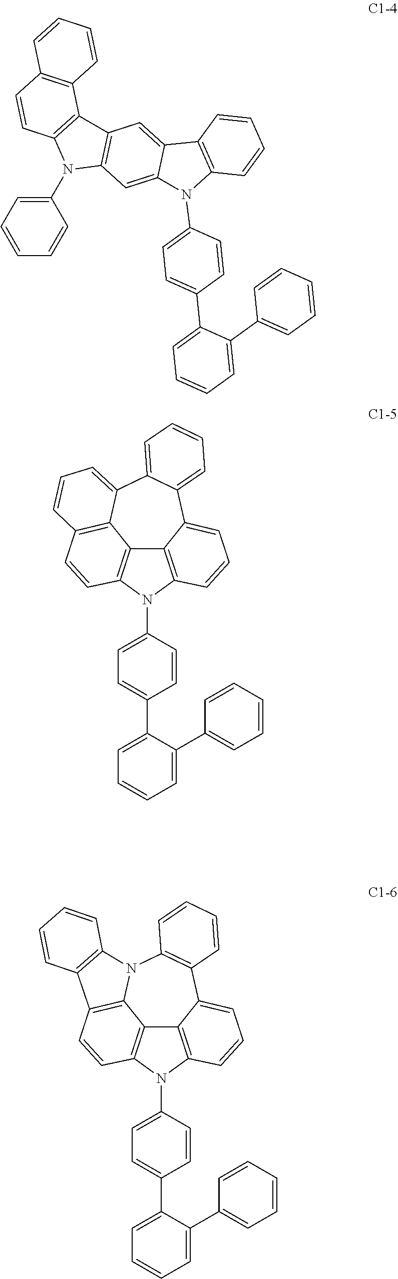

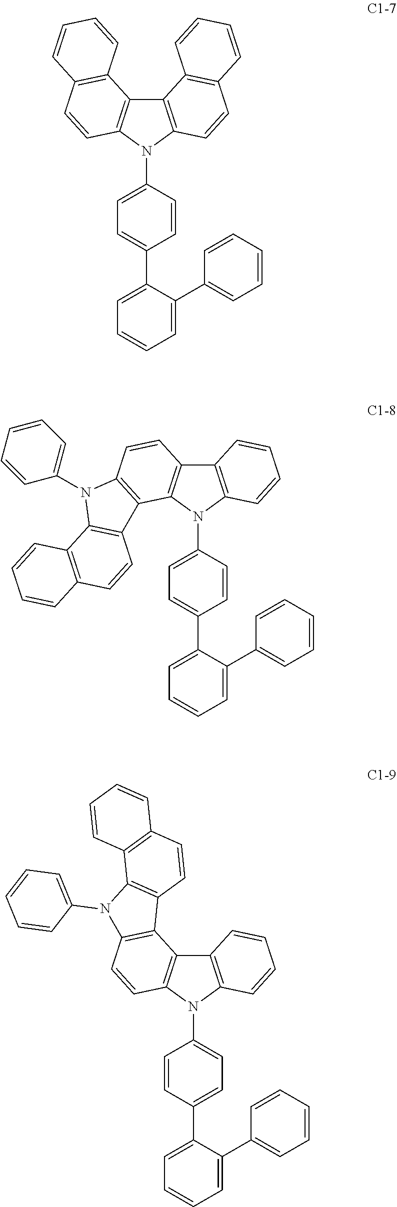 [SMILES: c1ccc(-c2ccccc2-c2ccc(-n3c4ccc5ccccc5c4c4c5ccccc5ccc43)cc2)cc1 ; c1ccc(-c2ccccc2-c2ccc(-n3c4ccccc4c4ccc5c(c6ccc7ccccc7c6n5-c5ccccc5)c43)cc2)cc1 ; c1ccc(-c2ccccc2-c2ccc(-n3c4ccccc4c4c5c6ccc7ccccc7c6n(-c6ccccc6)c5ccc43)cc2)cc1]

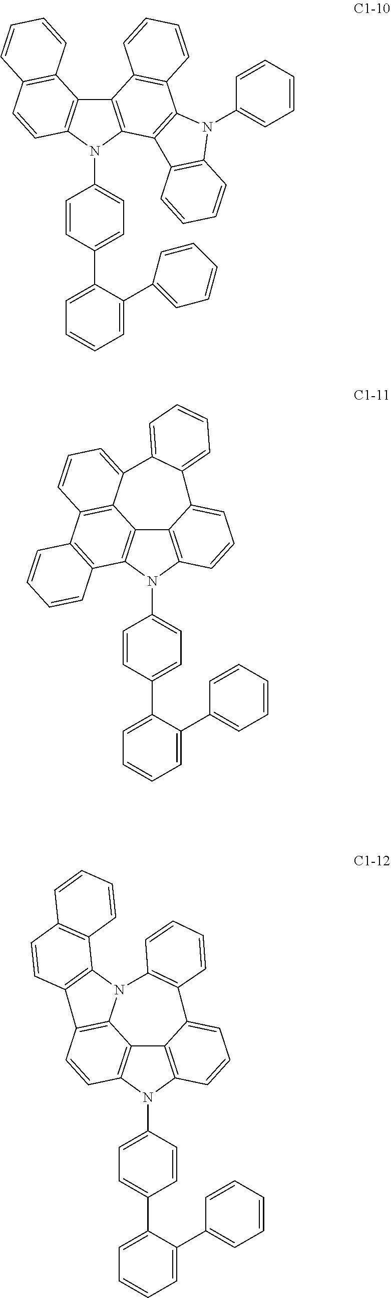 [SMILES: c1ccc(-c2ccccc2-c2ccc(-n3c4ccc5ccccc5c4c4c5ccccc5c5c(c6ccccc6n5-c5ccccc5)c43)cc2)cc1 ; c1ccc(-c2ccccc2-c2ccc(-n3c4cccc5c4c4c6c(cccc6c6ccccc6c43)-c3ccccc3-5)cc2)cc1 ; c1ccc(-c2ccccc2-c2ccc(-n3c4cccc5c6ccccc6n6c7c8ccccc8ccc7c7ccc3c(c54)c76)cc2)cc1]

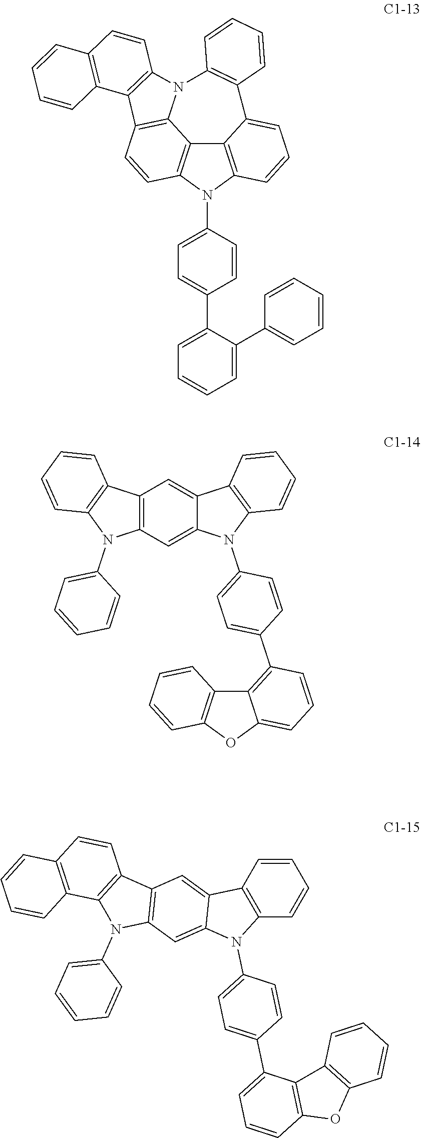 [SMILES: c1ccc(-c2ccccc2-c2ccc(-n3c4cccc5c6ccccc6n6c7ccc8ccccc8c7c7ccc3c(c54)c76)cc2)cc1 ; c1ccc(-n2c3ccccc3c3cc4c5ccccc5n(-c5ccc(-c6cccc7oc8ccccc8c67)cc5)c4cc32)cc1 ; c1ccc(-n2c3cc4c(cc3c3ccc5ccccc5c32)c2ccccc2n4-c2ccc(-c3cccc4oc5ccccc5c34)cc2)cc1]

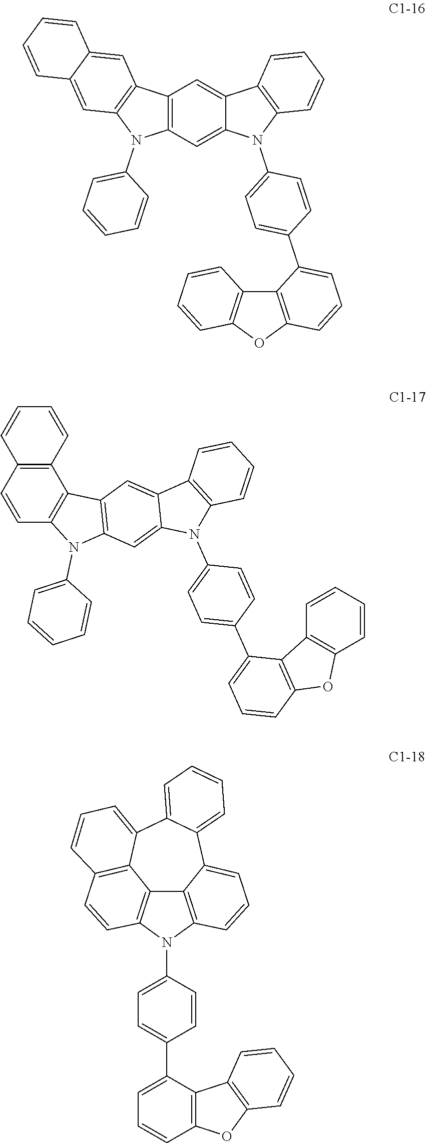 [SMILES: c1ccc(-n2c3cc4ccccc4cc3c3cc4c5ccccc5n(-c5ccc(-c6cccc7oc8ccccc8c67)cc5)c4cc32)cc1 ; c1ccc(-n2c3cc4c(cc3c3c5ccccc5ccc32)c2ccccc2n4-c2ccc(-c3cccc4oc5ccccc5c34)cc2)cc1 ; c1ccc2c(c1)-c1cccc3ccc4c(c13)c1c-2cccc1n4-c1ccc(-c2cccc3oc4ccccc4c23)cc1]

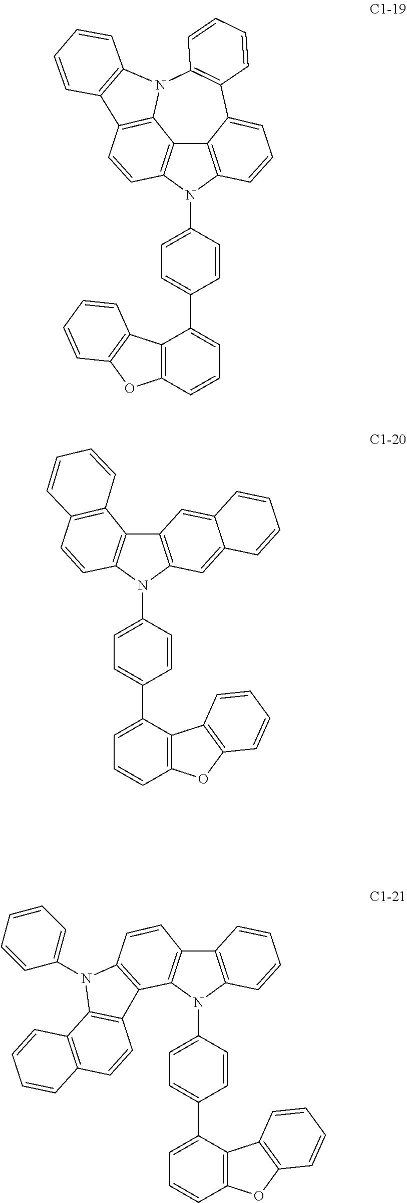 [SMILES: c1ccc2c(c1)oc1cccc(-c3ccc(-n4c5cccc6c7ccccc7n7c8ccccc8c8ccc4c(c65)c87)cc3)c12 ; c1ccc2cc3c(cc2c1)c1c2ccccc2ccc1n3-c1ccc(-c2cccc3oc4ccccc4c23)cc1 ; c1ccc(-n2c3ccc4c5ccccc5n(-c5ccc(-c6cccc7oc8ccccc8c67)cc5)c4c3c3ccc4ccccc4c32)cc1]

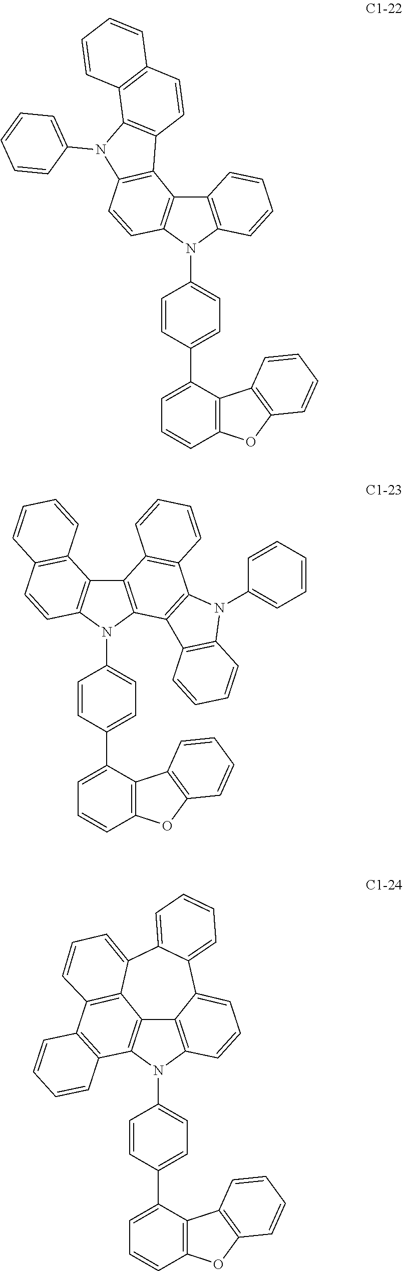 [SMILES: c1ccc(-n2c3ccc4c(c5ccccc5n4-c4ccc(-c5cccc6oc7ccccc7c56)cc4)c3c3ccc4ccccc4c32)cc1 ; c1ccc(-n2c3ccccc3c3c2c2ccccc2c2c4c5ccccc5ccc4n(-c4ccc(-c5cccc6oc7ccccc7c56)cc4)c23)cc1 ; c1ccc2c(c1)-c1cccc3c4ccccc4c4c(c13)c1c-2cccc1n4-c1ccc(-c2cccc3oc4ccccc4c23)cc1]

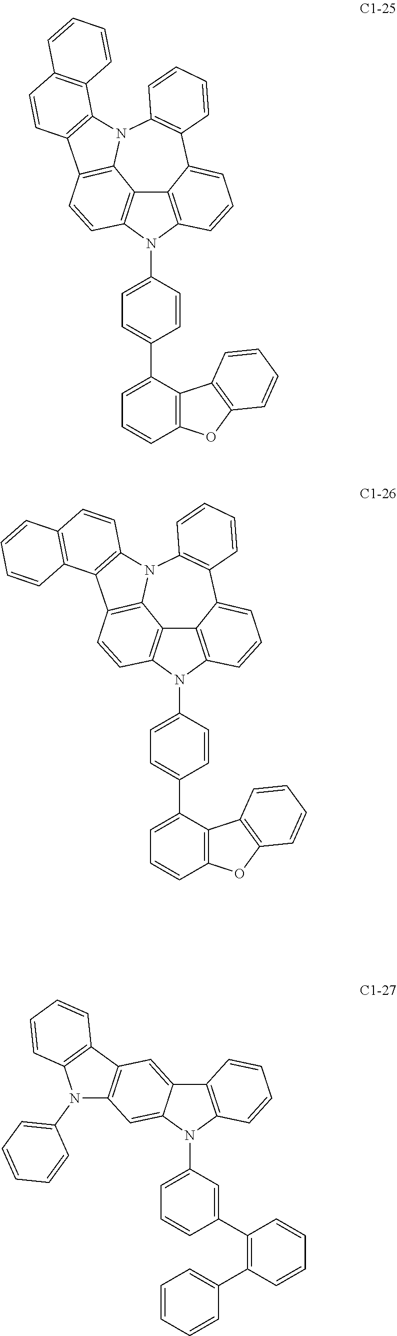 [SMILES: c1ccc2c(c1)ccc1c3ccc4c5c6c(cccc6n4-c4ccc(-c6cccc7oc8ccccc8c67)cc4)c4ccccc4n(c21)c35 ; c1ccc2c(c1)ccc1c2c2ccc3c4c5c(cccc5n3-c3ccc(-c5cccc6oc7ccccc7c56)cc3)c3ccccc3n1c24 ; c1ccc(-c2ccccc2-c2cccc(-n3c4ccccc4c4cc5c6ccccc6n(-c6ccccc6)c5cc43)c2)cc1]

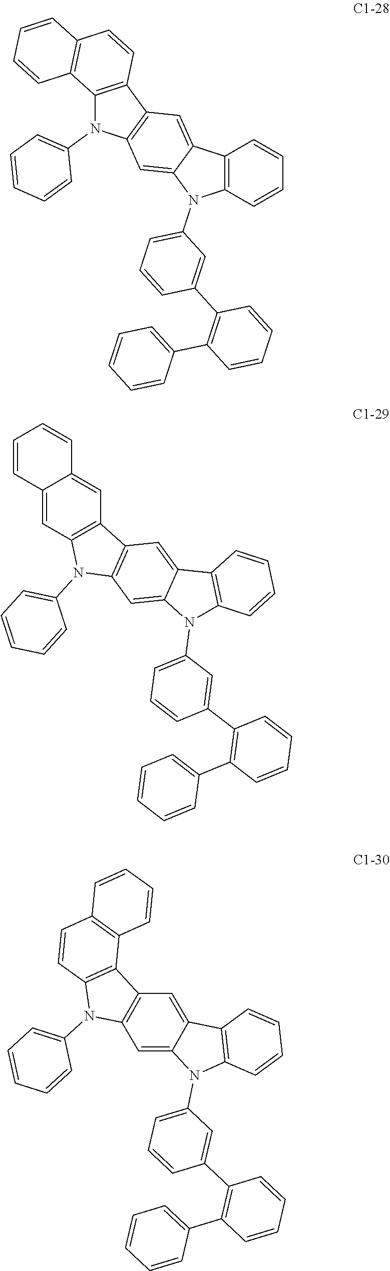 [SMILES: c1ccc(-c2ccccc2-c2cccc(-n3c4ccccc4c4cc5c6ccc7ccccc7c6n(-c6ccccc6)c5cc43)c2)cc1 ; c1ccc(-c2ccccc2-c2cccc(-n3c4ccccc4c4cc5c6cc7ccccc7cc6n(-c6ccccc6)c5cc43)c2)cc1 ; c1ccc(-c2ccccc2-c2cccc(-n3c4ccccc4c4cc5c6c7ccccc7ccc6n(-c6ccccc6)c5cc43)c2)cc1]

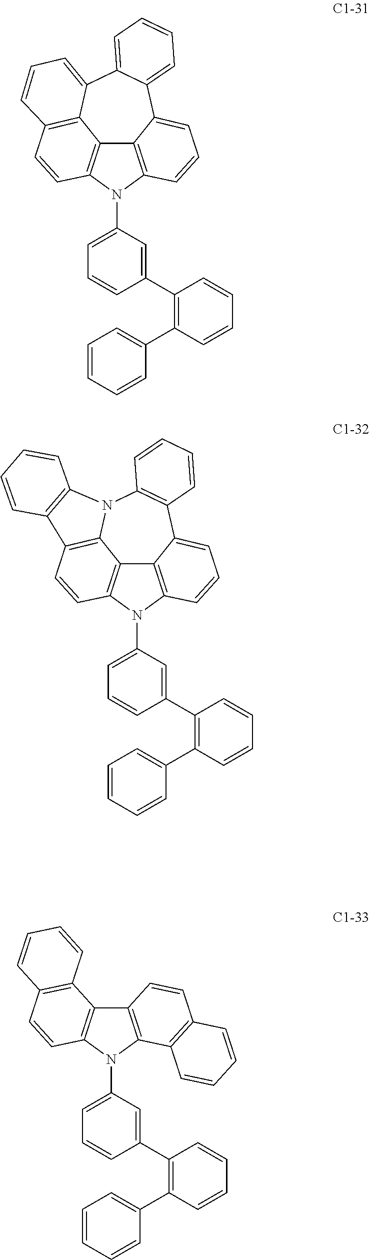 [SMILES: c1ccc(-c2ccccc2-c2cccc(-n3c4cccc5c4c4c6c(cccc6ccc43)-c3ccccc3-5)c2)cc1 ; c1ccc(-c2ccccc2-c2cccc(-n3c4cccc5c6ccccc6n6c7ccccc7c7ccc3c(c54)c76)c2)cc1 ; c1ccc(-c2ccccc2-c2cccc(-n3c4ccc5ccccc5c4c4ccc5ccccc5c43)c2)cc1]

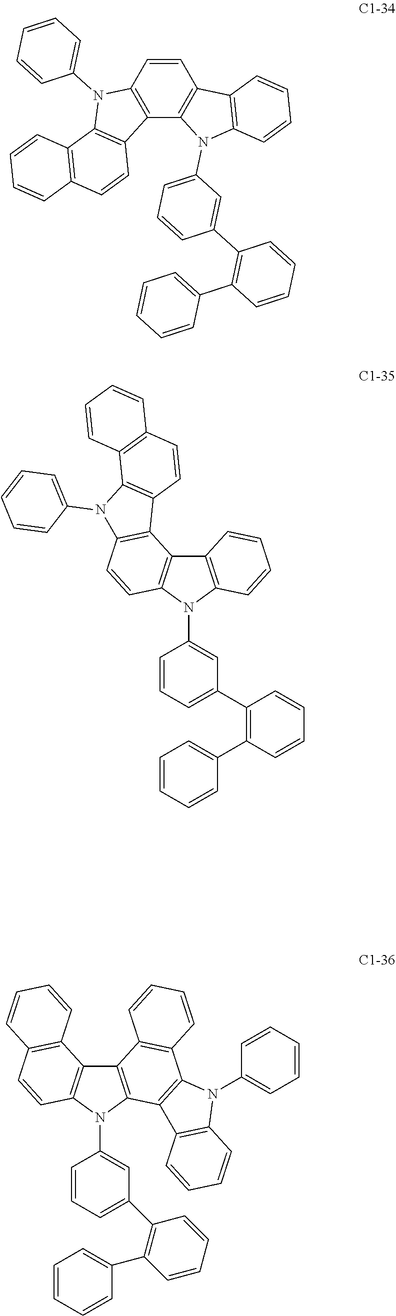 [SMILES: c1ccc(-c2ccccc2-c2cccc(-n3c4ccccc4c4ccc5c(c6ccc7ccccc7c6n5-c5ccccc5)c43)c2)cc1 ; c1ccc(-c2ccccc2-c2cccc(-n3c4ccccc4c4c5c6ccc7ccccc7c6n(-c6ccccc6)c5ccc43)c2)cc1 ; c1ccc(-c2ccccc2-c2cccc(-n3c4ccc5ccccc5c4c4c5ccccc5c5c(c6ccccc6n5-c5ccccc5)c43)c2)cc1]

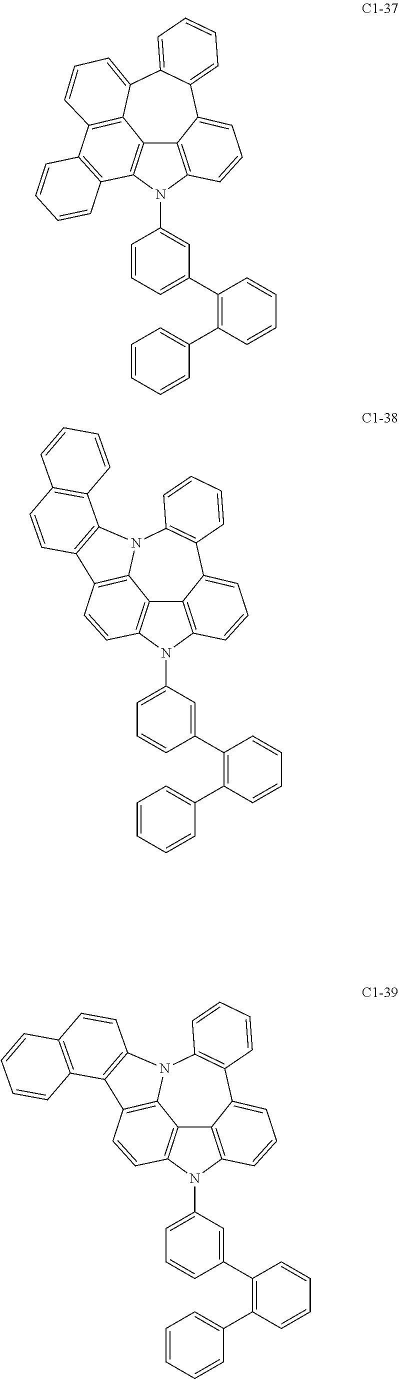 [SMILES: c1ccc(-c2ccccc2-c2cccc(-n3c4cccc5c4c4c6c(cccc6c6ccccc6c43)-c3ccccc3-5)c2)cc1 ; c1ccc(-c2ccccc2-c2cccc(-n3c4cccc5c6ccccc6n6c7c8ccccc8ccc7c7ccc3c(c54)c76)c2)cc1 ; c1ccc(-c2ccccc2-c2cccc(-n3c4cccc5c6ccccc6n6c7ccc8ccccc8c7c7ccc3c(c54)c76)c2)cc1]

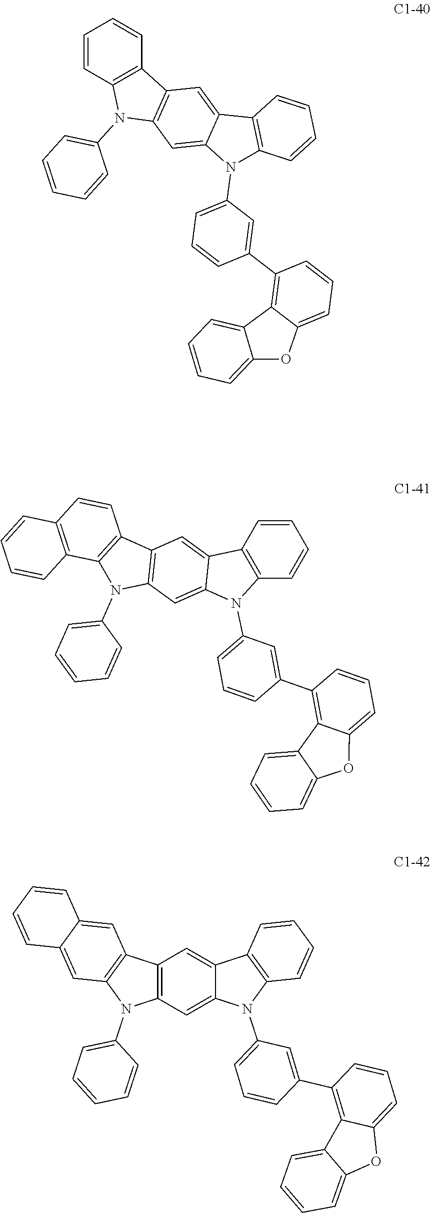 [SMILES: c1ccc(-n2c3ccccc3c3cc4c5ccccc5n(-c5cccc(-c6cccc7oc8ccccc8c67)c5)c4cc32)cc1 ; c1ccc(-n2c3cc4c(cc3c3ccc5ccccc5c32)c2ccccc2n4-c2cccc(-c3cccc4oc5ccccc5c34)c2)cc1 ; c1ccc(-n2c3cc4ccccc4cc3c3cc4c5ccccc5n(-c5cccc(-c6cccc7oc8ccccc8c67)c5)c4cc32)cc1]

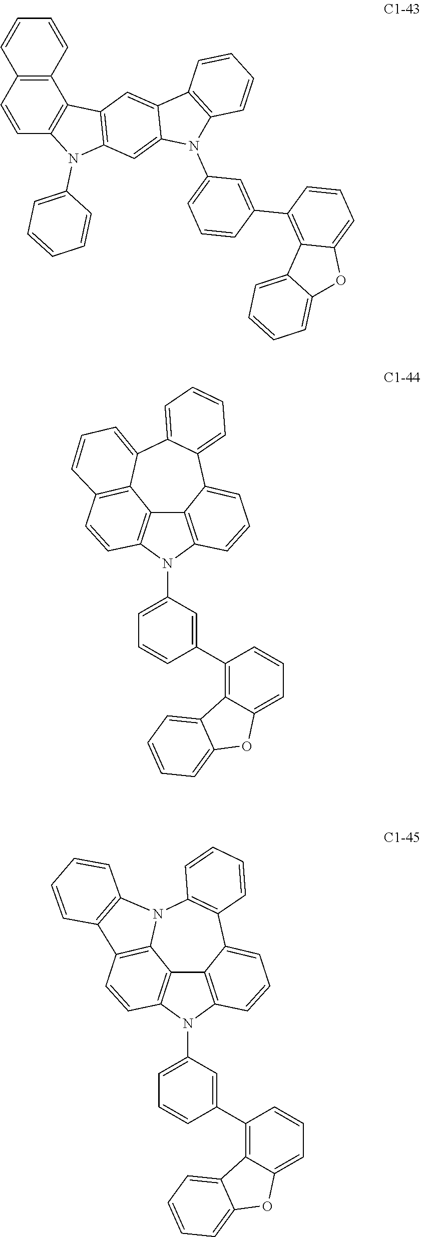 [SMILES: c1ccc(-n2c3cc4c(cc3c3c5ccccc5ccc32)c2ccccc2n4-c2cccc(-c3cccc4oc5ccccc5c34)c2)cc1 ; c1cc(-c2cccc3oc4ccccc4c23)cc(-n2c3cccc4c3c3c5c(cccc5ccc32)-c2ccccc2-4)c1 ; c1cc(-c2cccc3oc4ccccc4c23)cc(-n2c3cccc4c5ccccc5n5c6ccccc6c6ccc2c(c43)c65)c1]

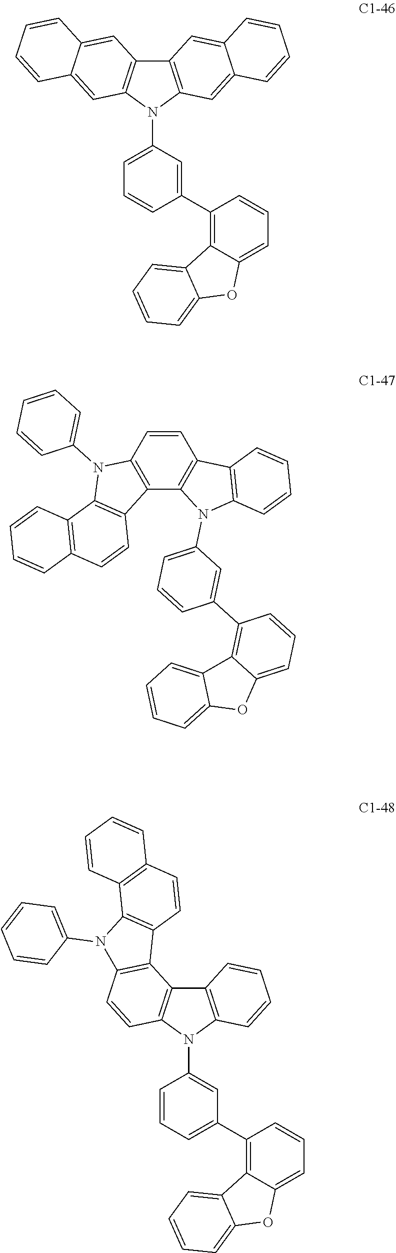 [SMILES: c1cc(-c2cccc3oc4ccccc4c23)cc(-n2c3cc4ccccc4cc3c3cc4ccccc4cc32)c1 ; c1ccc(-n2c3ccc4c5ccccc5n(-c5cccc(-c6cccc7oc8ccccc8c67)c5)c4c3c3ccc4ccccc4c32)cc1 ; c1ccc(-n2c3ccc4c(c5ccccc5n4-c4cccc(-c5cccc6oc7ccccc7c56)c4)c3c3ccc4ccccc4c32)cc1]

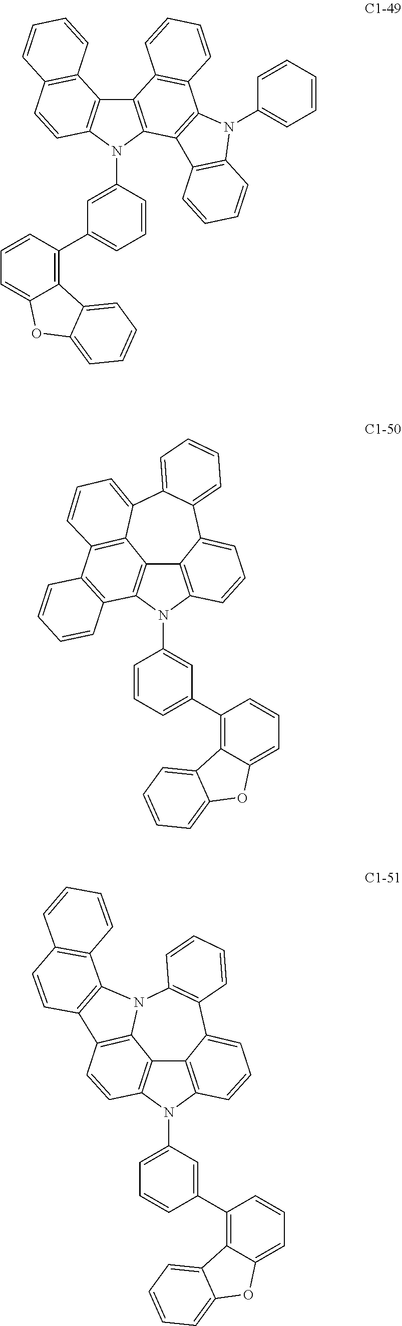 [SMILES: c1ccc(-n2c3ccccc3c3c2c2ccccc2c2c4c5ccccc5ccc4n(-c4cccc(-c5cccc6oc7ccccc7c56)c4)c23)cc1 ; c1cc(-c2cccc3oc4ccccc4c23)cc(-n2c3cccc4c3c3c5c(cccc5c5ccccc5c32)-c2ccccc2-4)c1 ; c1cc(-c2cccc3oc4ccccc4c23)cc(-n2c3cccc4c5ccccc5n5c6c7ccccc7ccc6c6ccc2c(c43)c65)c1]

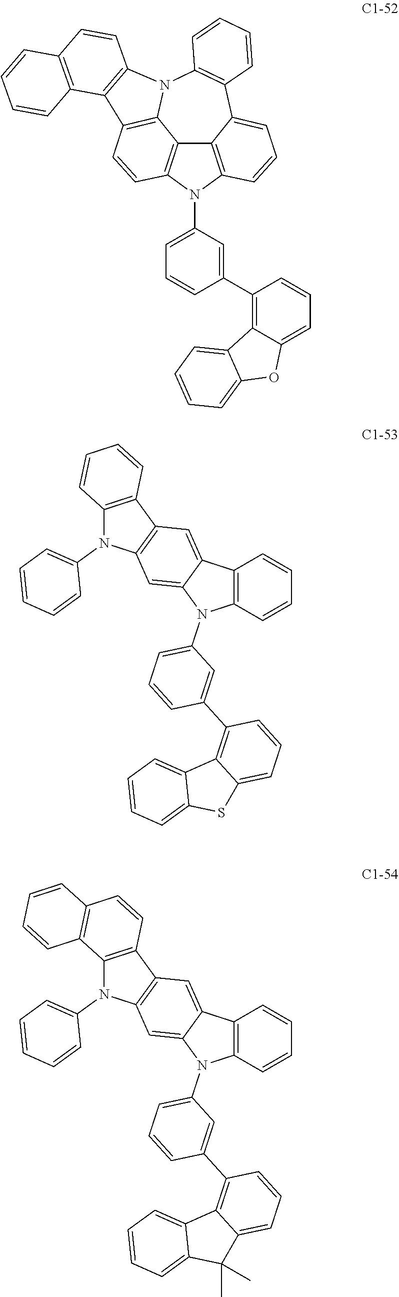 [SMILES: c1cc(-c2cccc3oc4ccccc4c23)cc(-n2c3cccc4c5ccccc5n5c6ccc7ccccc7c6c6ccc2c(c43)c65)c1 ; c1ccc(-n2c3ccccc3c3cc4c5ccccc5n(-c5cccc(-c6cccc7sc8ccccc8c67)c5)c4cc32)cc1 ; CC1(C)c2ccccc2-c2c(-c3cccc(-n4c5ccccc5c5cc6c7ccc8ccccc8c7n(-c7ccccc7)c6cc54)c3)cccc21]

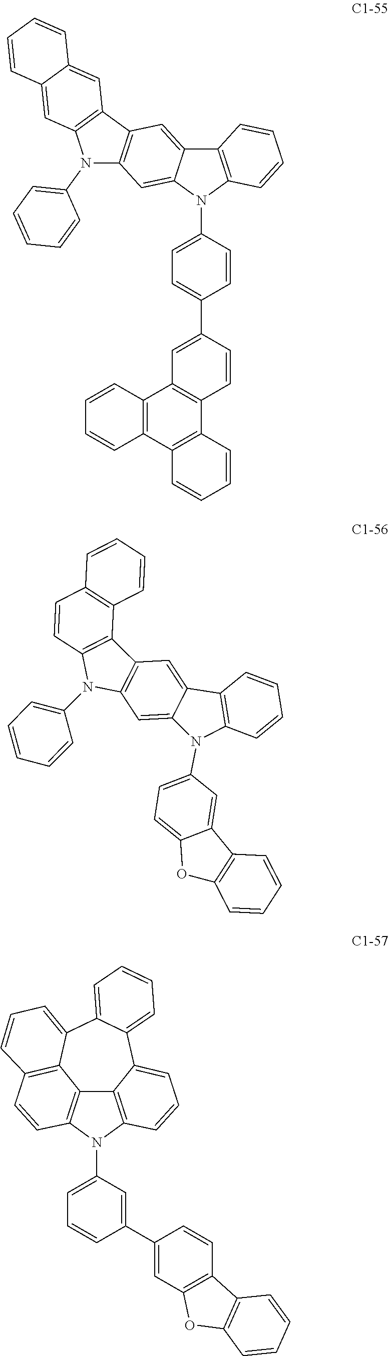 [SMILES: c1ccc(-n2c3cc4ccccc4cc3c3cc4c5ccccc5n(-c5ccc(-c6ccc7c8ccccc8c8ccccc8c7c6)cc5)c4cc32)cc1 ; c1ccc(-n2c3cc4c(cc3c3c5ccccc5ccc32)c2ccccc2n4-c2ccc3oc4ccccc4c3c2)cc1 ; c1cc(-c2ccc3c(c2)oc2ccccc23)cc(-n2c3cccc4c3c3c5c(cccc5ccc32)-c2ccccc2-4)c1]

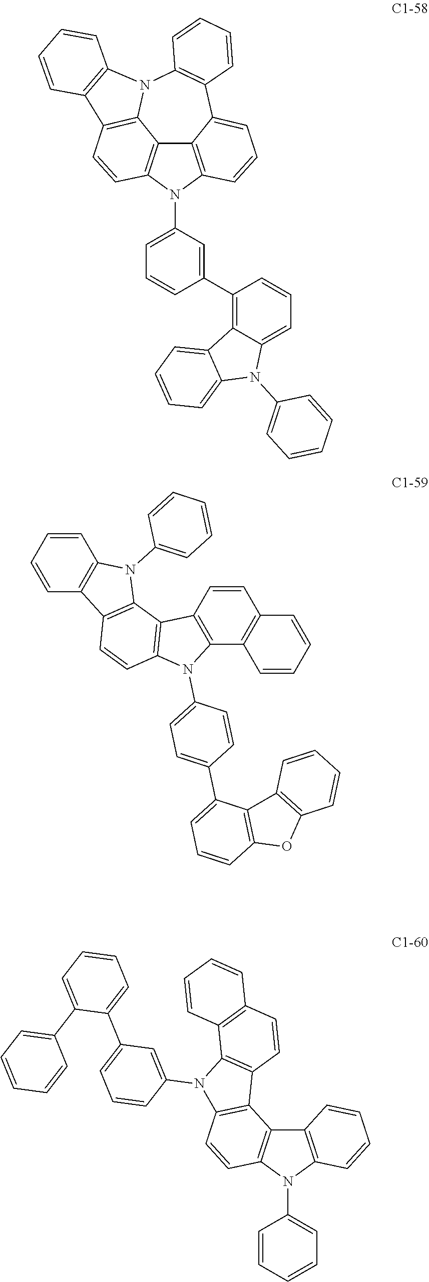 [SMILES: c1ccc(-n2c3ccccc3c3c(-c4cccc(-n5c6cccc7c8ccccc8n8c9ccccc9c9ccc5c(c76)c98)c4)cccc32)cc1 ; c1ccc(-n2c3ccccc3c3ccc4c(c5ccc6ccccc6c5n4-c4ccc(-c5cccc6oc7ccccc7c56)cc4)c32)cc1 ; c1ccc(-c2ccccc2-c2cccc(-n3c4ccc5c(c6ccccc6n5-c5ccccc5)c4c4ccc5ccccc5c43)c2)cc1]

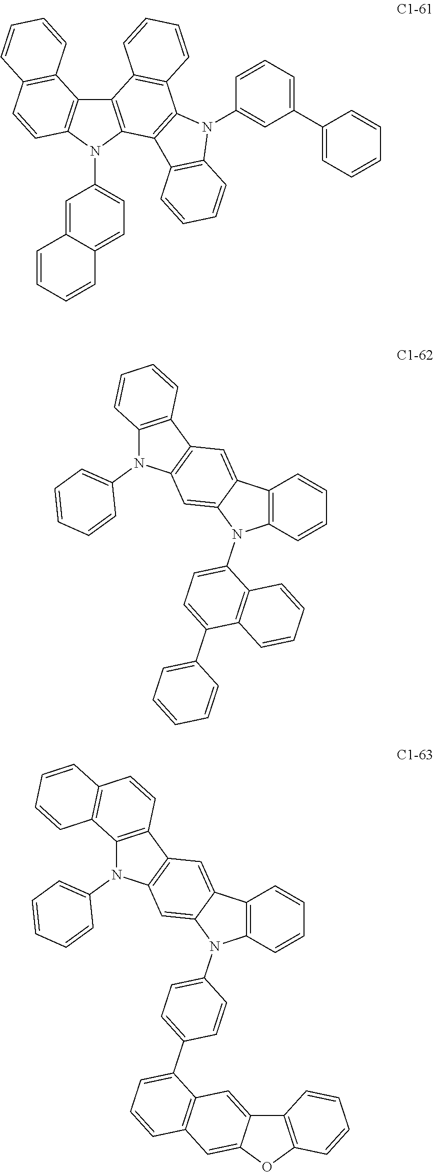 [SMILES: c1ccc(-c2cccc(-n3c4ccccc4c4c3c3ccccc3c3c5c6ccccc6ccc5n(-c5ccc6ccccc6c5)c34)c2)cc1 ; c1ccc(-c2ccc(-n3c4ccccc4c4cc5c6ccccc6n(-c6ccccc6)c5cc43)c3ccccc23)cc1 ; c1ccc(-n2c3cc4c(cc3c3ccc5ccccc5c32)c2ccccc2n4-c2ccc(-c3cccc4cc5oc6ccccc6c5cc34)cc2)cc1]

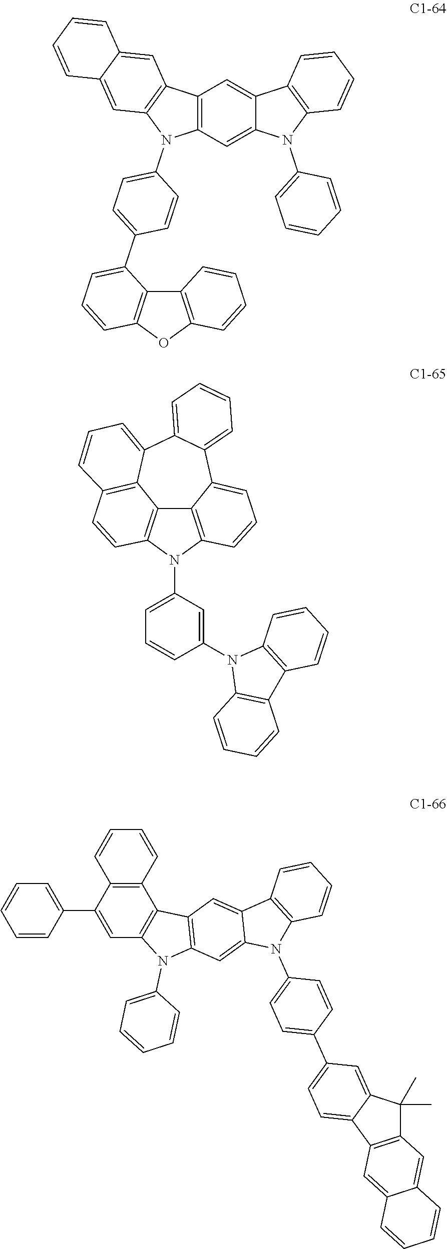 [SMILES: c1ccc(-n2c3ccccc3c3cc4c5cc6ccccc6cc5n(-c5ccc(-c6cccc7oc8ccccc8c67)cc5)c4cc32)cc1 ; c1cc(-n2c3ccccc3c3ccccc32)cc(-n2c3cccc4c3c3c5c(cccc5ccc32)-c2ccccc2-4)c1 ; CC1(C)c2cc(-c3ccc(-n4c5ccccc5c5cc6c7c8ccccc8c(-c8ccccc8)cc7n(-c7ccccc7)c6cc54)cc3)ccc2-c2cc3ccccc3cc21]

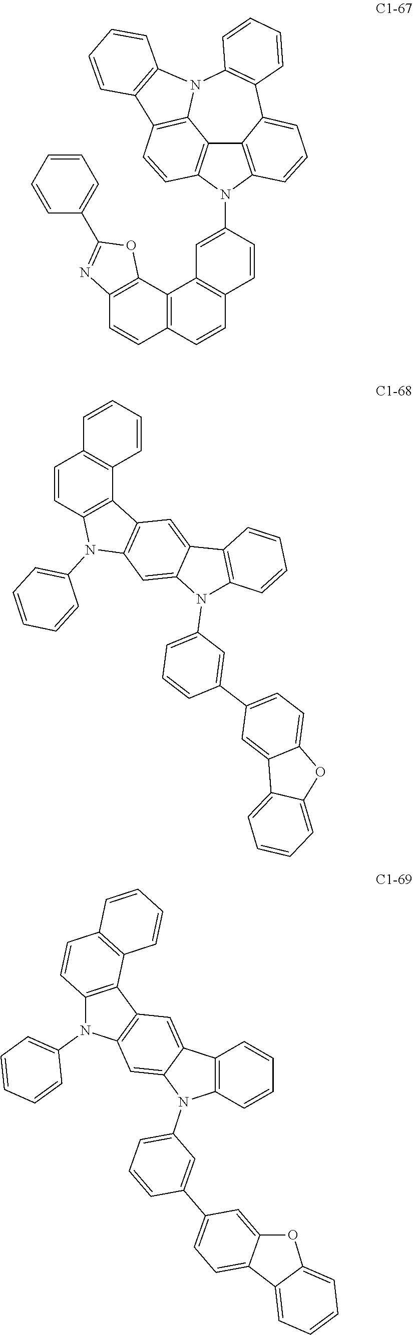 [SMILES: c1ccc(-c2nc3ccc4ccc5ccc(-n6c7cccc8c9ccccc9n9c%10ccccc%10c%10ccc6c(c87)c%109)cc5c4c3o2)cc1 ; c1ccc(-n2c3cc4c(cc3c3c5ccccc5ccc32)c2ccccc2n4-c2cccc(-c3ccc4oc5ccccc5c4c3)c2)cc1 ; c1ccc(-n2c3cc4c(cc3c3c5ccccc5ccc32)c2ccccc2n4-c2cccc(-c3ccc4c(c3)oc3ccccc34)c2)cc1]

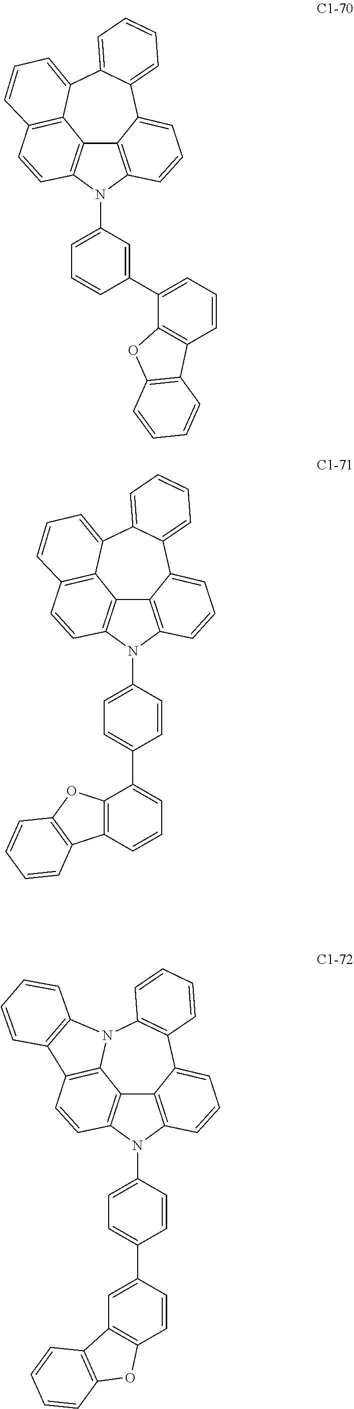

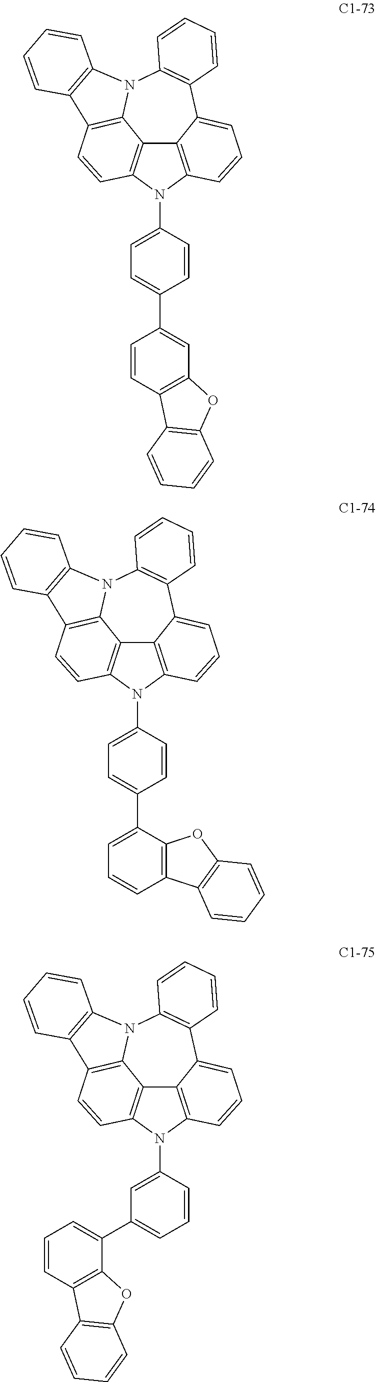

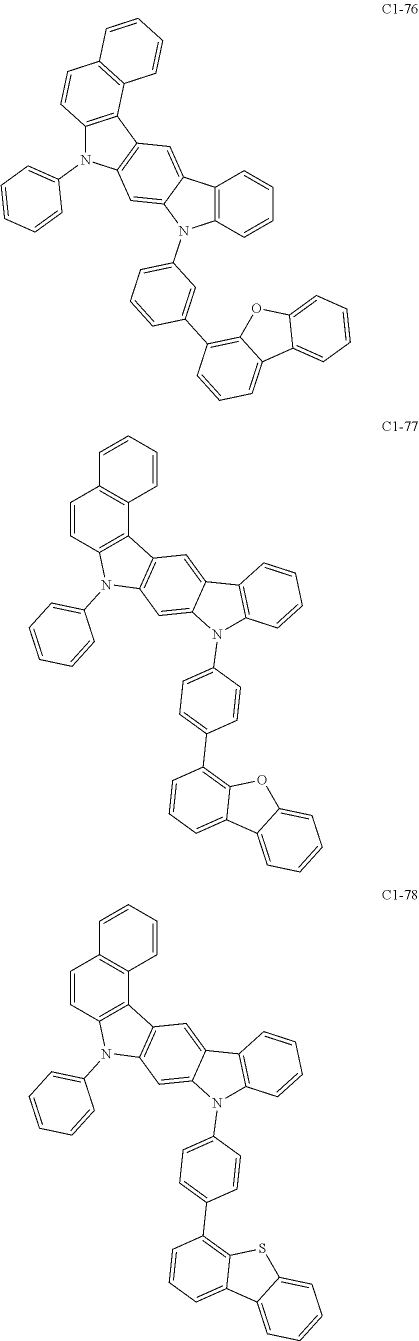 [SMILES: c1ccc(-n2c3cc4c(cc3c3c5ccccc5ccc32)c2ccccc2n4-c2cccc(-c3cccc4c3oc3ccccc34)c2)cc1 ; c1ccc(-n2c3cc4c(cc3c3c5ccccc5ccc32)c2ccccc2n4-c2ccc(-c3cccc4c3oc3ccccc34)cc2)cc1 ; c1ccc(-n2c3cc4c(cc3c3c5ccccc5ccc32)c2ccccc2n4-c2ccc(-c3cccc4c3sc3ccccc34)cc2)cc1]

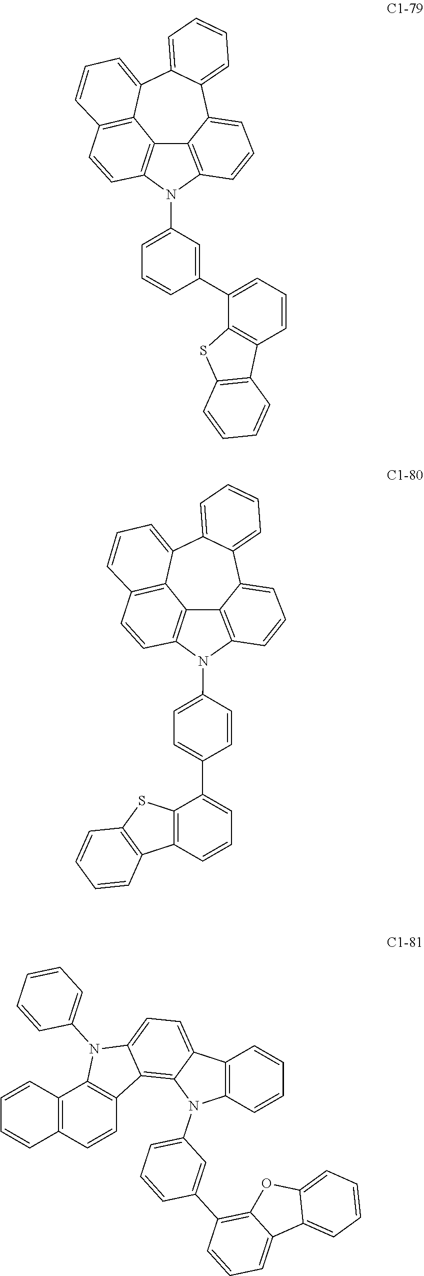

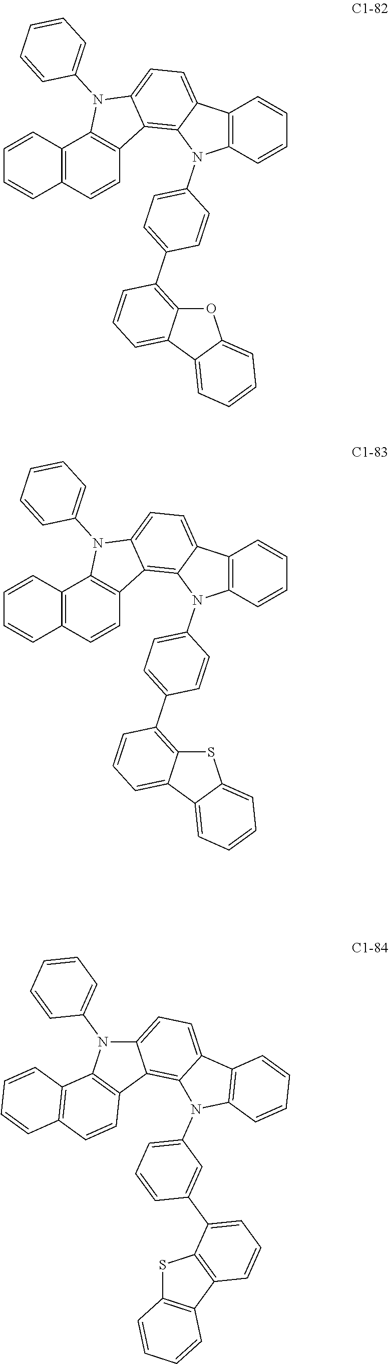 [SMILES: c1ccc(-n2c3ccc4c5ccccc5n(-c5ccc(-c6cccc7c6oc6ccccc67)cc5)c4c3c3ccc4ccccc4c32)cc1 ; c1ccc(-n2c3ccc4c5ccccc5n(-c5ccc(-c6cccc7c6sc6ccccc67)cc5)c4c3c3ccc4ccccc4c32)cc1 ; c1ccc(-n2c3ccc4c5ccccc5n(-c5cccc(-c6cccc7c6sc6ccccc67)c5)c4c3c3ccc4ccccc4c32)cc1]

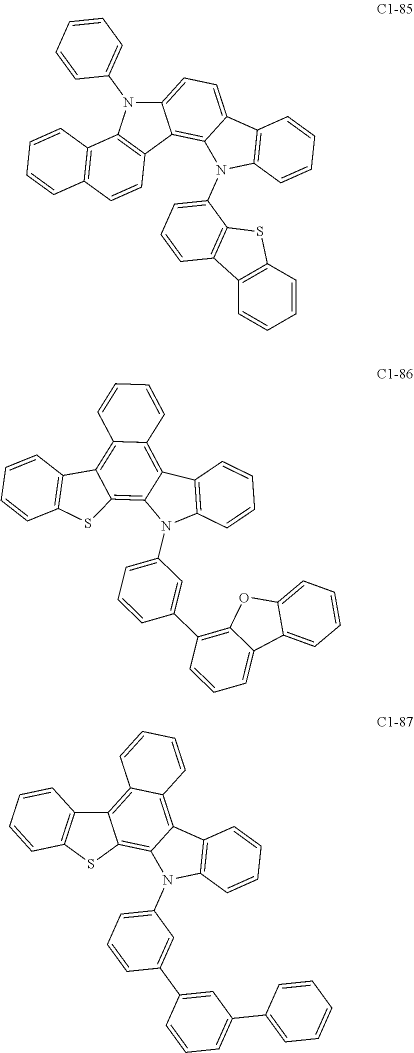 [SMILES: c1ccc(-n2c3ccc4c5ccccc5n(-c5cccc6c5sc5ccccc56)c4c3c3ccc4ccccc4c32)cc1 ; c1cc(-c2cccc3c2oc2ccccc23)cc(-n2c3ccccc3c3c4ccccc4c4c5ccccc5sc4c32)c1 ; c1ccc(-c2cccc(-c3cccc(-n4c5ccccc5c5c6ccccc6c6c7ccccc7sc6c54)c3)c2)cc1]

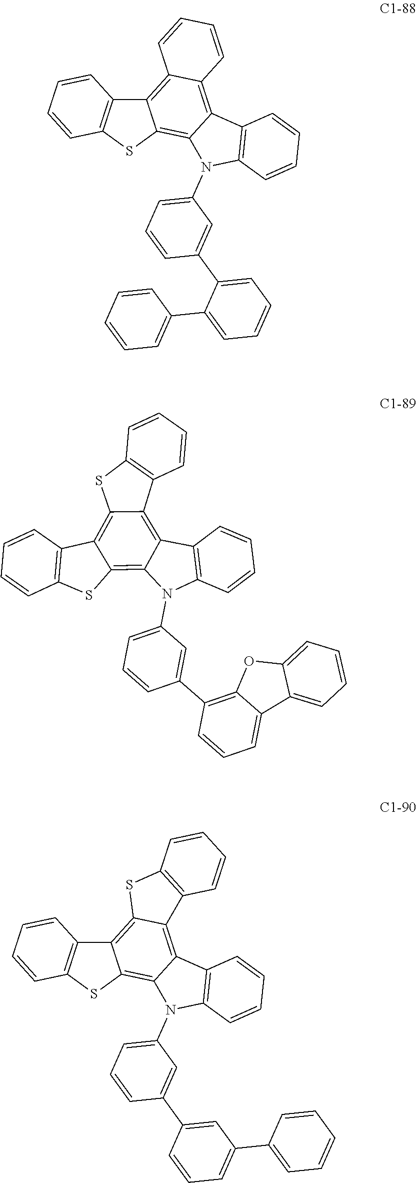 [SMILES: c1ccc(-c2ccccc2-c2cccc(-n3c4ccccc4c4c5ccccc5c5c6ccccc6sc5c43)c2)cc1 ; c1cc(-c2cccc3c2oc2ccccc23)cc(-n2c3ccccc3c3c4c5ccccc5sc4c4c5ccccc5sc4c32)c1 ; c1ccc(-c2cccc(-c3cccc(-n4c5ccccc5c5c6c7ccccc7sc6c6c7ccccc7sc6c54)c3)c2)cc1]

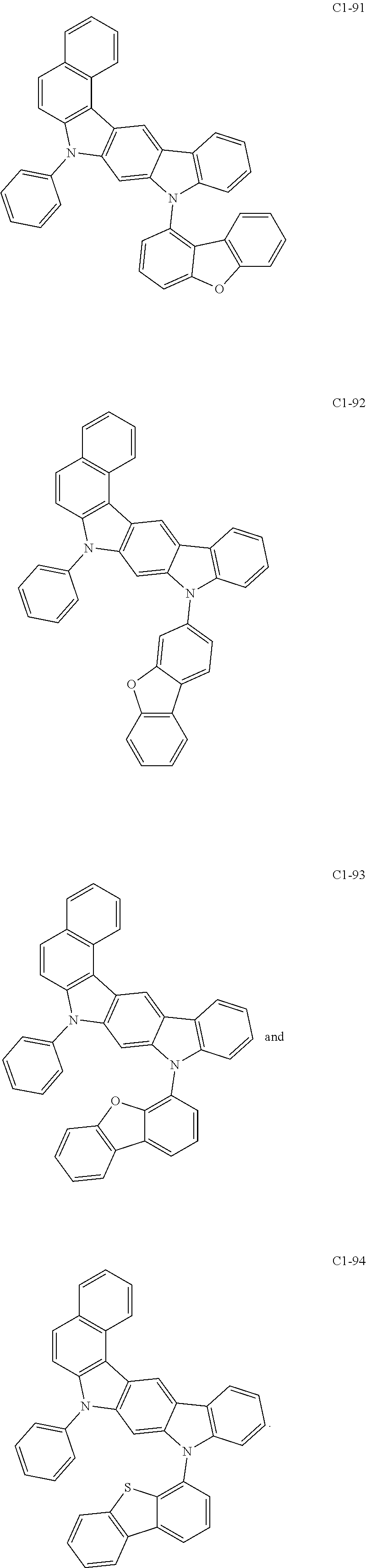 [SMILES: c1ccc(-n2c3cc4c(cc3c3c5ccccc5ccc32)c2ccccc2n4-c2cccc3oc4ccccc4c23)cc1 ; c1ccc(-n2c3cc4c(cc3c3c5ccccc5ccc32)c2ccccc2n4-c2ccc3c(c2)oc2ccccc23)cc1 ; c1ccc(-n2c3cc4c(cc3c3c5ccccc5ccc32)c2ccccc2n4-c2cccc3c2oc2ccccc23)cc1 ; c1ccc(-n2c3cc4c(cc3c3c5ccccc5ccc32)c2ccccc2n4-c2cccc3c2sc2ccccc23)cc1]

8. The plurality of host materials according to claim 1, wherein the compound represented by formula 2 is at least one selected from the group consisting of the following compounds:

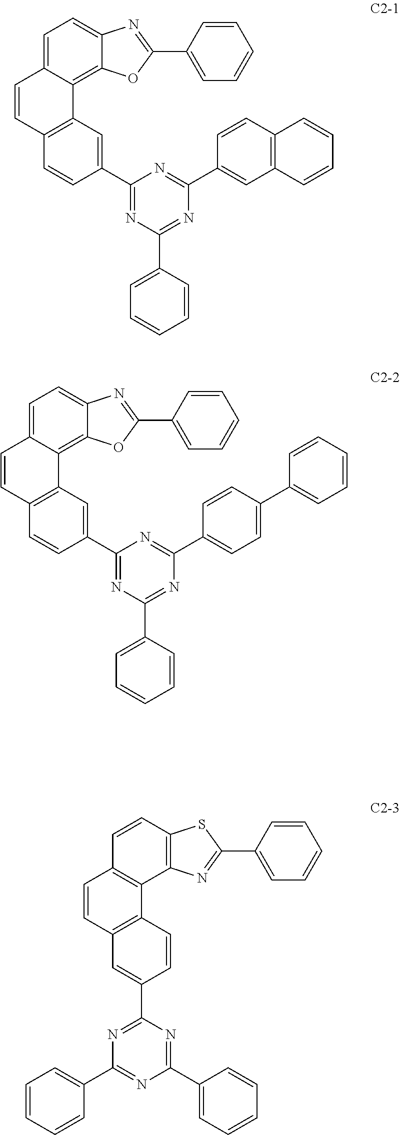 [SMILES: c1ccc(-c2nc(-c3ccc4ccccc4c3)nc(-c3ccc4ccc5ccc6nc(-c7ccccc7)oc6c5c4c3)n2)cc1 ; c1ccc(-c2ccc(-c3nc(-c4ccccc4)nc(-c4ccc5ccc6ccc7nc(-c8ccccc8)oc7c6c5c4)n3)cc2)cc1 ; c1ccc(-c2nc(-c3ccccc3)nc(-c3ccc4c(ccc5ccc6sc(-c7ccccc7)nc6c54)c3)n2)cc1]

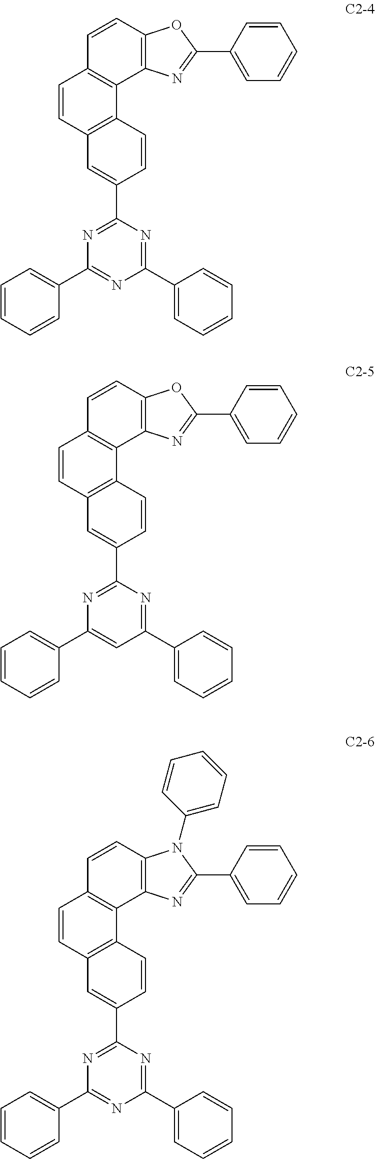 [SMILES: c1ccc(-c2nc(-c3ccccc3)nc(-c3ccc4c(ccc5ccc6oc(-c7ccccc7)nc6c54)c3)n2)cc1 ; c1ccc(-c2cc(-c3ccccc3)nc(-c3ccc4c(ccc5ccc6oc(-c7ccccc7)nc6c54)c3)n2)cc1 ; c1ccc(-c2nc(-c3ccccc3)nc(-c3ccc4c(ccc5ccc6c(nc(-c7ccccc7)n6-c6ccccc6)c54)c3)n2)cc1]

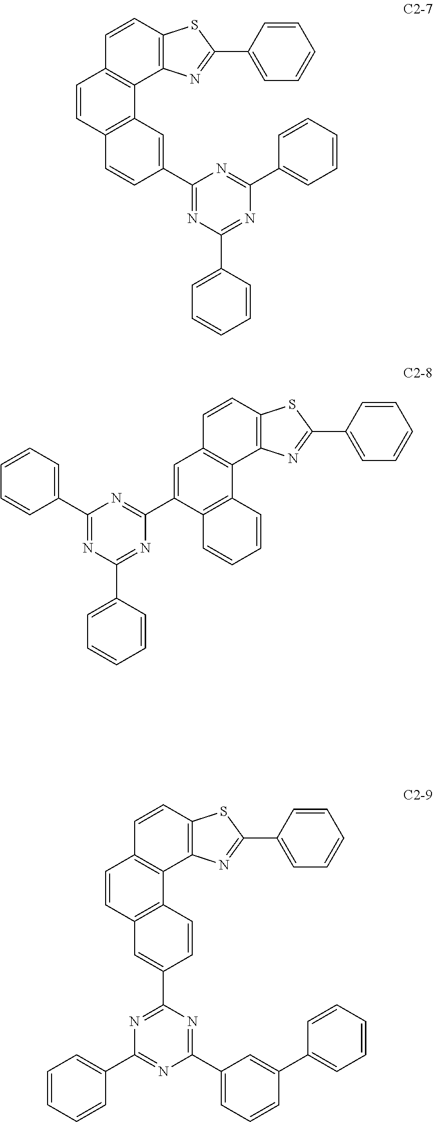 [SMILES: c1ccc(-c2nc(-c3ccccc3)nc(-c3ccc4ccc5ccc6sc(-c7ccccc7)nc6c5c4c3)n2)cc1 ; c1ccc(-c2nc(-c3ccccc3)nc(-c3cc4ccc5sc(-c6ccccc6)nc5c4c4ccccc34)n2)cc1 ; c1ccc(-c2cccc(-c3nc(-c4ccccc4)nc(-c4ccc5c(ccc6ccc7sc(-c8ccccc8)nc7c65)c4)n3)c2)cc1]

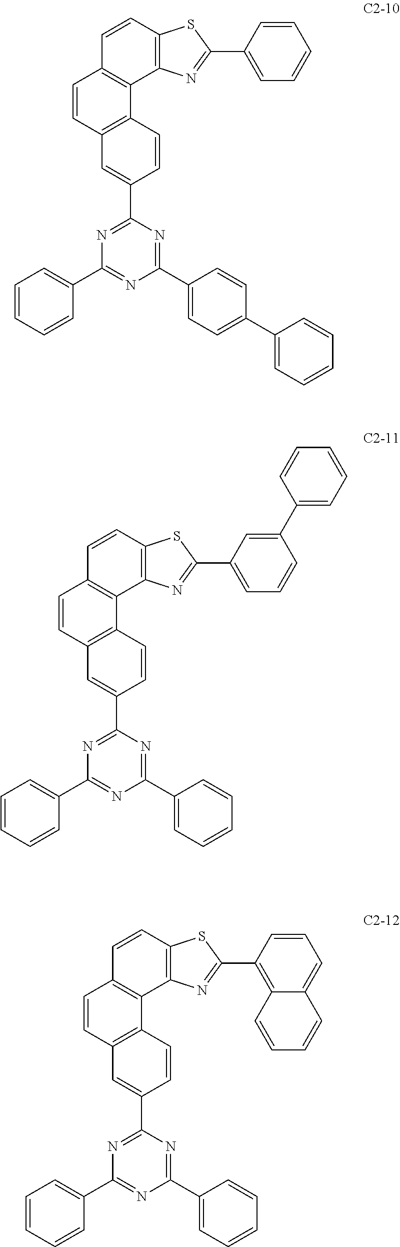 [SMILES: c1ccc(-c2ccc(-c3nc(-c4ccccc4)nc(-c4ccc5c(ccc6ccc7sc(-c8ccccc8)nc7c65)c4)n3)cc2)cc1 ; c1ccc(-c2cccc(-c3nc4c(ccc5ccc6cc(-c7nc(-c8ccccc8)nc(-c8ccccc8)n7)ccc6c54)s3)c2)cc1 ; c1ccc(-c2nc(-c3ccccc3)nc(-c3ccc4c(ccc5ccc6sc(-c7cccc8ccccc78)nc6c54)c3)n2)cc1]

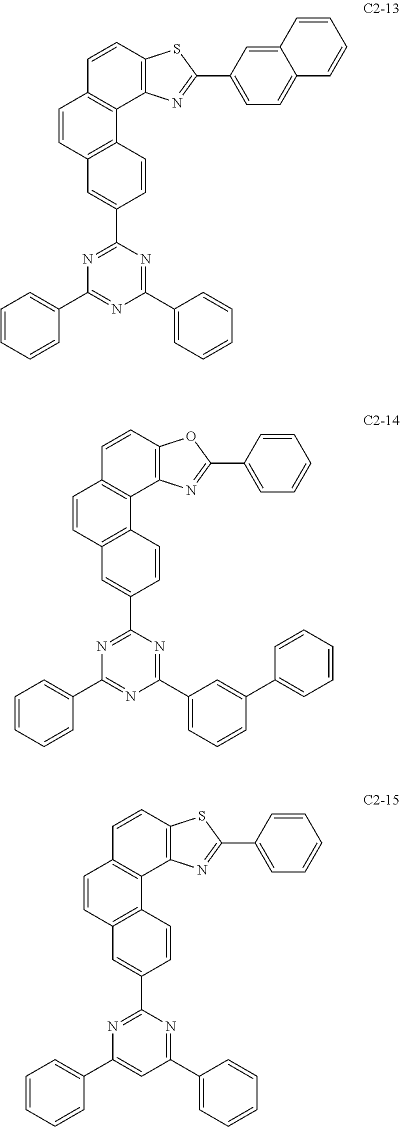 [SMILES: c1ccc(-c2nc(-c3ccccc3)nc(-c3ccc4c(ccc5ccc6sc(-c7ccc8ccccc8c7)nc6c54)c3)n2)cc1 ; c1ccc(-c2cccc(-c3nc(-c4ccccc4)nc(-c4ccc5c(ccc6ccc7oc(-c8ccccc8)nc7c65)c4)n3)c2)cc1 ; c1ccc(-c2cc(-c3ccccc3)nc(-c3ccc4c(ccc5ccc6sc(-c7ccccc7)nc6c54)c3)n2)cc1]

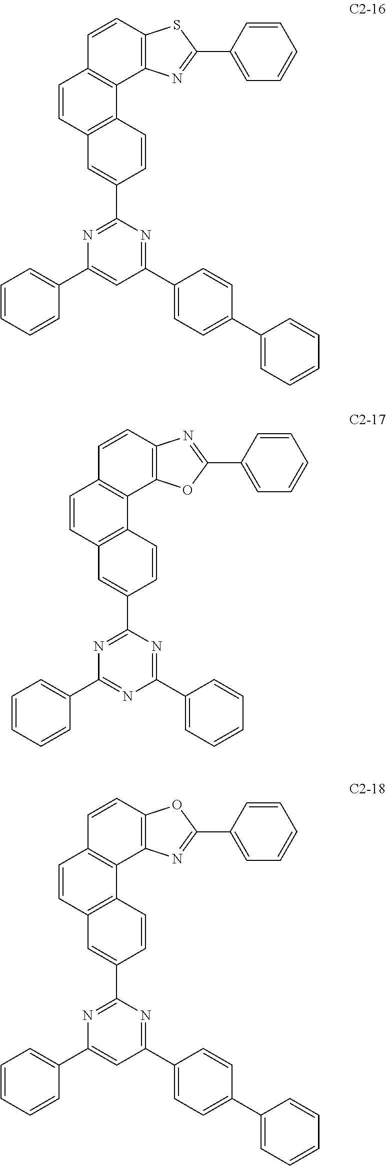 [SMILES: c1ccc(-c2ccc(-c3cc(-c4ccccc4)nc(-c4ccc5c(ccc6ccc7sc(-c8ccccc8)nc7c65)c4)n3)cc2)cc1 ; c1ccc(-c2nc(-c3ccccc3)nc(-c3ccc4c(ccc5ccc6nc(-c7ccccc7)oc6c54)c3)n2)cc1 ; c1ccc(-c2ccc(-c3cc(-c4ccccc4)nc(-c4ccc5c(ccc6ccc7oc(-c8ccccc8)nc7c65)c4)n3)cc2)cc1]

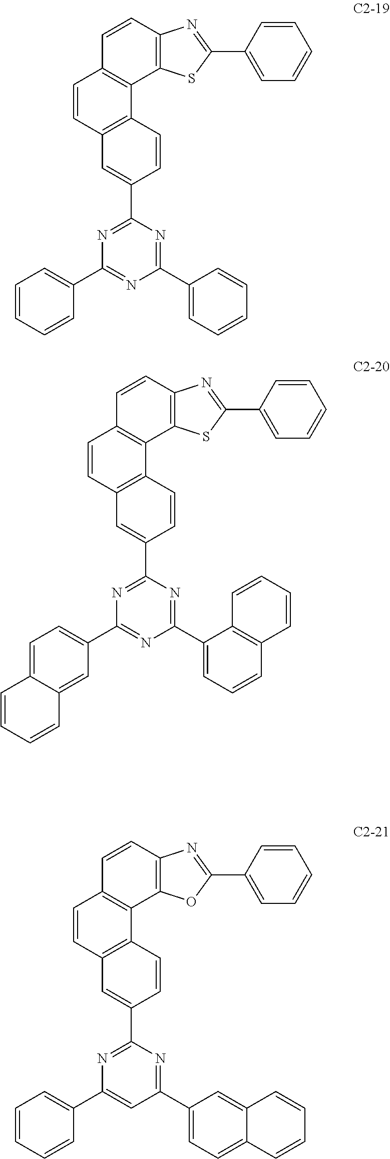 [SMILES: c1ccc(-c2nc(-c3ccccc3)nc(-c3ccc4c(ccc5ccc6nc(-c7ccccc7)sc6c54)c3)n2)cc1 ; c1ccc(-c2nc3ccc4ccc5cc(-c6nc(-c7ccc8ccccc8c7)nc(-c7cccc8ccccc78)n6)ccc5c4c3s2)cc1 ; c1ccc(-c2cc(-c3ccc4ccccc4c3)nc(-c3ccc4c(ccc5ccc6nc(-c7ccccc7)oc6c54)c3)n2)cc1]

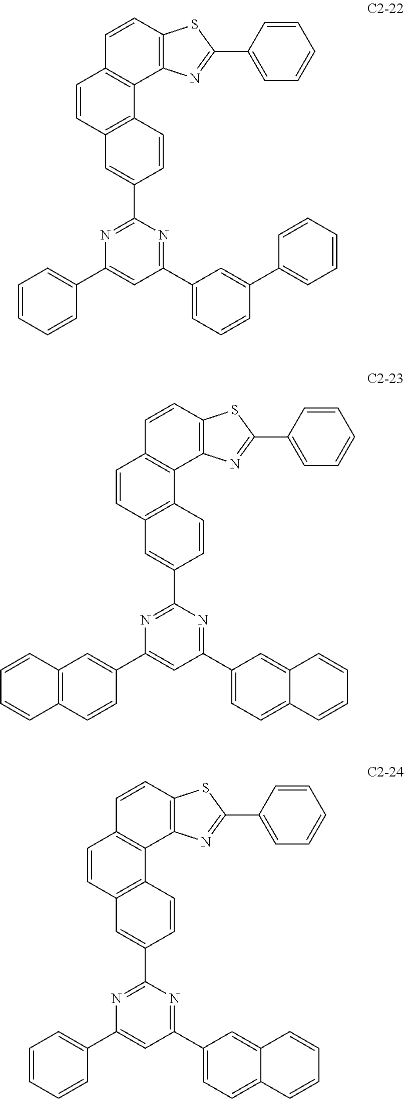 [SMILES: c1ccc(-c2cccc(-c3cc(-c4ccccc4)nc(-c4ccc5c(ccc6ccc7sc(-c8ccccc8)nc7c65)c4)n3)c2)cc1 ; c1ccc(-c2nc3c(ccc4ccc5cc(-c6nc(-c7ccc8ccccc8c7)cc(-c7ccc8ccccc8c7)n6)ccc5c43)s2)cc1 ; c1ccc(-c2cc(-c3ccc4ccccc4c3)nc(-c3ccc4c(ccc5ccc6sc(-c7ccccc7)nc6c54)c3)n2)cc1]

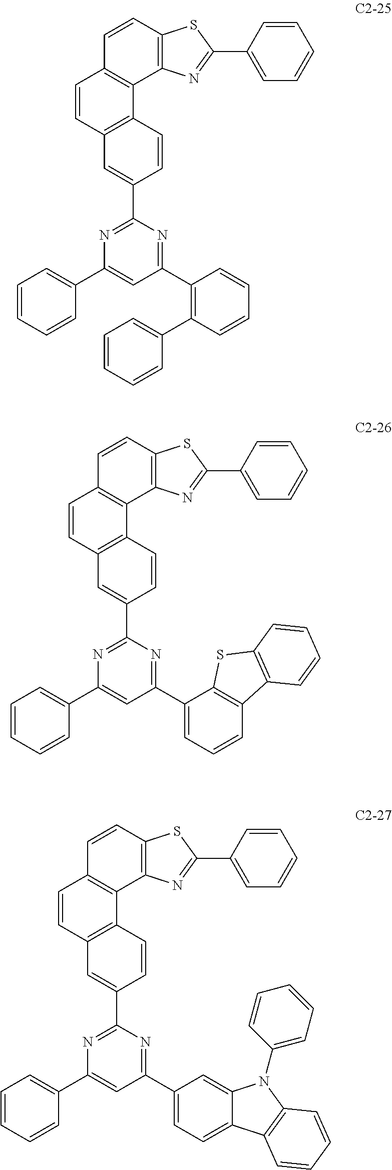 [SMILES: c1ccc(-c2cc(-c3ccccc3-c3ccccc3)nc(-c3ccc4c(ccc5ccc6sc(-c7ccccc7)nc6c54)c3)n2)cc1 ; c1ccc(-c2cc(-c3cccc4c3sc3ccccc34)nc(-c3ccc4c(ccc5ccc6sc(-c7ccccc7)nc6c54)c3)n2)cc1 ; c1ccc(-c2cc(-c3ccc4c5ccccc5n(-c5ccccc5)c4c3)nc(-c3ccc4c(ccc5ccc6sc(-c7ccccc7)nc6c54)c3)n2)cc1]

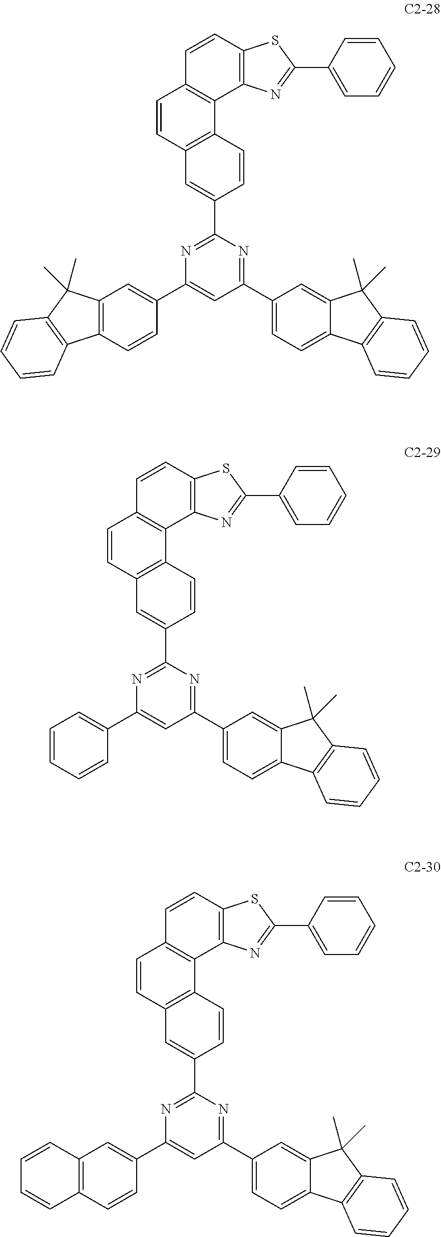 [SMILES: CC1(C)c2ccccc2-c2ccc(-c3cc(-c4ccc5c(c4)C(C)(C)c4ccccc4-5)nc(-c4ccc5c(ccc6ccc7sc(-c8ccccc8)nc7c65)c4)n3)cc21 ; CC1(C)c2ccccc2-c2ccc(-c3cc(-c4ccccc4)nc(-c4ccc5c(ccc6ccc7sc(-c8ccccc8)nc7c65)c4)n3)cc21 ; CC1(C)c2ccccc2-c2ccc(-c3cc(-c4ccc5ccccc5c4)nc(-c4ccc5c(ccc6ccc7sc(-c8ccccc8)nc7c65)c4)n3)cc21]

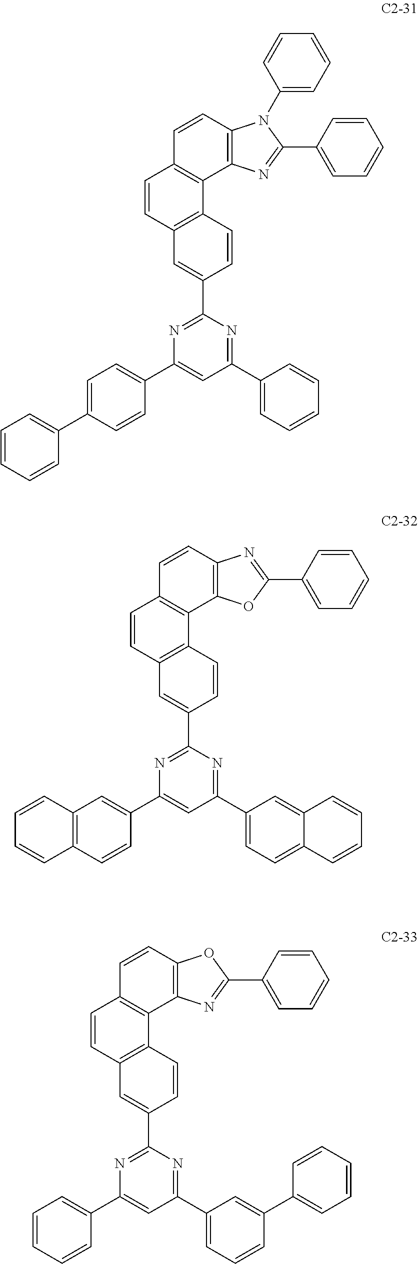 [SMILES: c1ccc(-c2ccc(-c3cc(-c4ccccc4)nc(-c4ccc5c(ccc6ccc7c(nc(-c8ccccc8)n7-c7ccccc7)c65)c4)n3)cc2)cc1 ; c1ccc(-c2nc3ccc4ccc5cc(-c6nc(-c7ccc8ccccc8c7)cc(-c7ccc8ccccc8c7)n6)ccc5c4c3o2)cc1 ; c1ccc(-c2cccc(-c3cc(-c4ccccc4)nc(-c4ccc5c(ccc6ccc7oc(-c8ccccc8)nc7c65)c4)n3)c2)cc1]

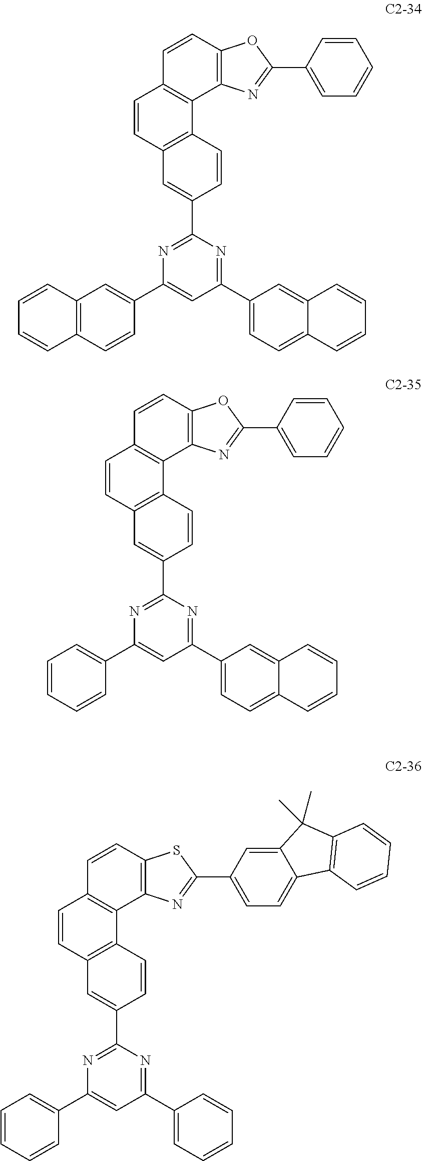 [SMILES: c1ccc(-c2nc3c(ccc4ccc5cc(-c6nc(-c7ccc8ccccc8c7)cc(-c7ccc8ccccc8c7)n6)ccc5c43)o2)cc1 ; c1ccc(-c2cc(-c3ccc4ccccc4c3)nc(-c3ccc4c(ccc5ccc6oc(-c7ccccc7)nc6c54)c3)n2)cc1 ; CC1(C)c2ccccc2-c2ccc(-c3nc4c(ccc5ccc6cc(-c7nc(-c8ccccc8)cc(-c8ccccc8)n7)ccc6c54)s3)cc21]

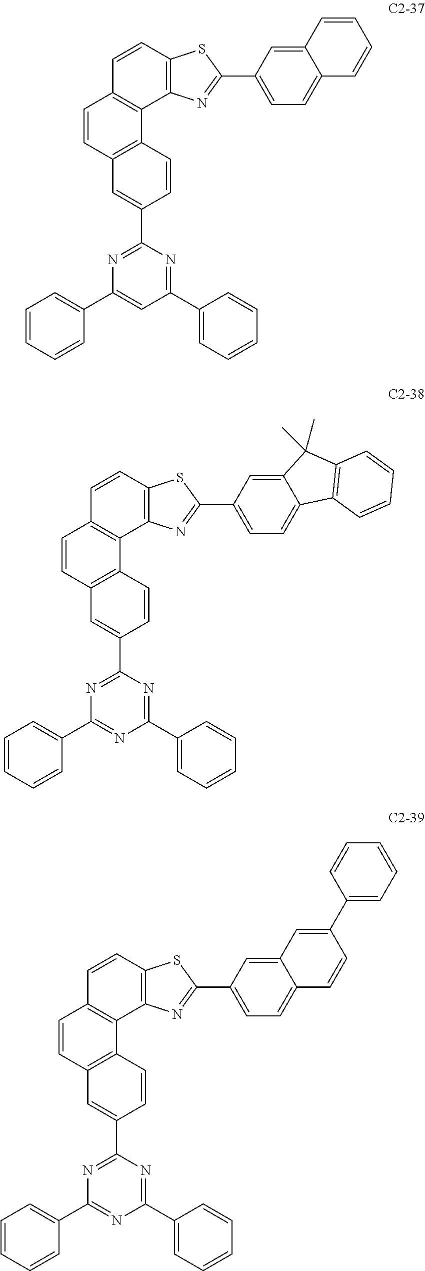 [SMILES: c1ccc(-c2cc(-c3ccccc3)nc(-c3ccc4c(ccc5ccc6sc(-c7ccc8ccccc8c7)nc6c54)c3)n2)cc1 ; CC1(C)c2ccccc2-c2ccc(-c3nc4c(ccc5ccc6cc(-c7nc(-c8ccccc8)nc(-c8ccccc8)n7)ccc6c54)s3)cc21 ; c1ccc(-c2ccc3ccc(-c4nc5c(ccc6ccc7cc(-c8nc(-c9ccccc9)nc(-c9ccccc9)n8)ccc7c65)s4)cc3c2)cc1]

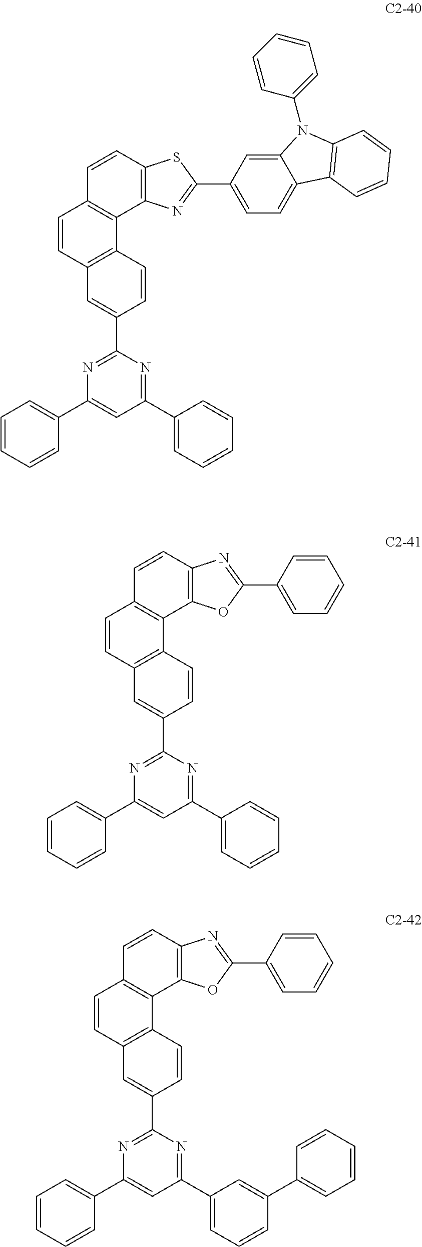 [SMILES: c1ccc(-c2cc(-c3ccccc3)nc(-c3ccc4c(ccc5ccc6sc(-c7ccc8c9ccccc9n(-c9ccccc9)c8c7)nc6c54)c3)n2)cc1 ; c1ccc(-c2cc(-c3ccccc3)nc(-c3ccc4c(ccc5ccc6nc(-c7ccccc7)oc6c54)c3)n2)cc1 ; c1ccc(-c2cccc(-c3cc(-c4ccccc4)nc(-c4ccc5c(ccc6ccc7nc(-c8ccccc8)oc7c65)c4)n3)c2)cc1]

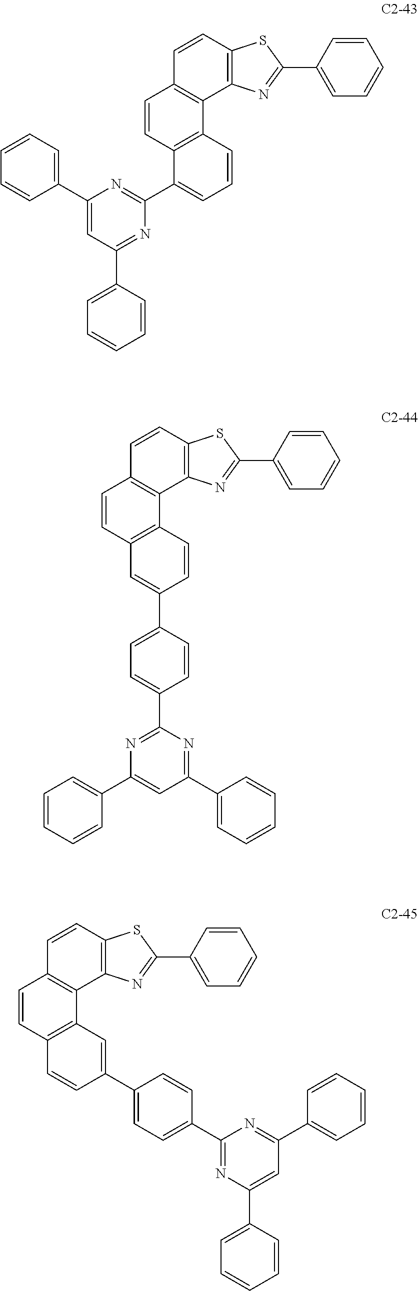 [SMILES: c1ccc(-c2cc(-c3ccccc3)nc(-c3cccc4c3ccc3ccc5sc(-c6ccccc6)nc5c34)n2)cc1 ; c1ccc(-c2cc(-c3ccccc3)nc(-c3ccc(-c4ccc5c(ccc6ccc7sc(-c8ccccc8)nc7c65)c4)cc3)n2)cc1 ; c1ccc(-c2cc(-c3ccccc3)nc(-c3ccc(-c4ccc5ccc6ccc7sc(-c8ccccc8)nc7c6c5c4)cc3)n2)cc1]

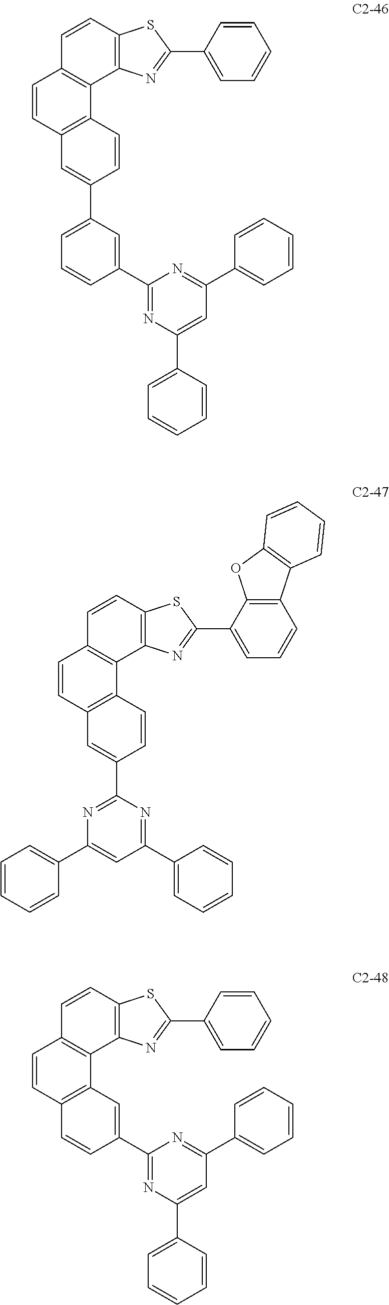 [SMILES: c1ccc(-c2cc(-c3ccccc3)nc(-c3cccc(-c4ccc5c(ccc6ccc7sc(-c8ccccc8)nc7c65)c4)c3)n2)cc1 ; c1ccc(-c2cc(-c3ccccc3)nc(-c3ccc4c(ccc5ccc6sc(-c7cccc8c7oc7ccccc78)nc6c54)c3)n2)cc1 ; c1ccc(-c2cc(-c3ccccc3)nc(-c3ccc4ccc5ccc6sc(-c7ccccc7)nc6c5c4c3)n2)cc1]

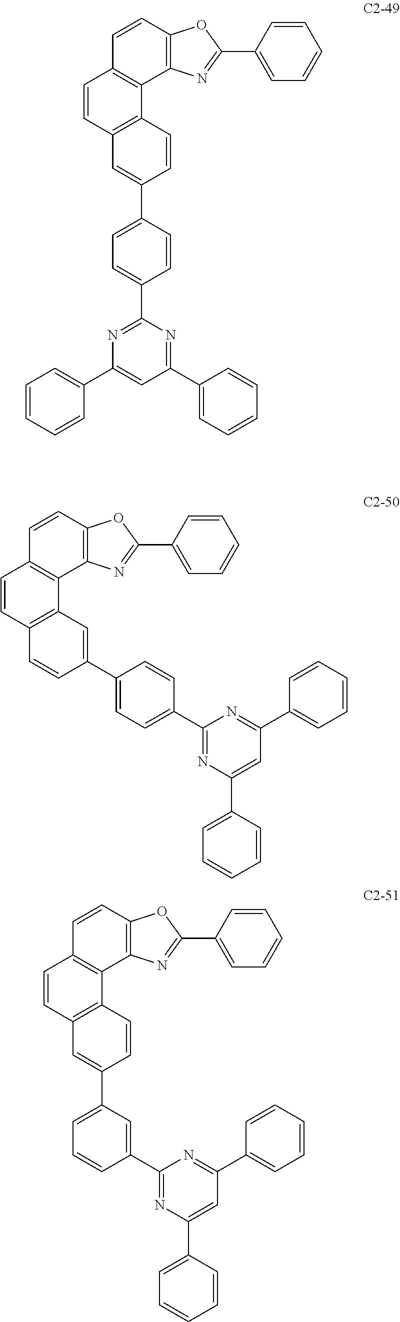

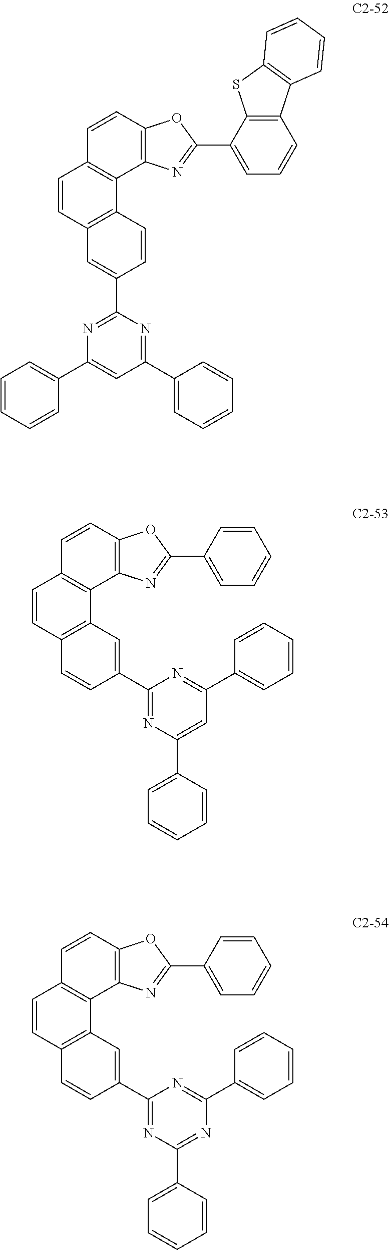 [SMILES: c1ccc(-c2cc(-c3ccccc3)nc(-c3ccc4c(ccc5ccc6oc(-c7cccc8c7sc7ccccc78)nc6c54)c3)n2)cc1 ; c1ccc(-c2cc(-c3ccccc3)nc(-c3ccc4ccc5ccc6oc(-c7ccccc7)nc6c5c4c3)n2)cc1 ; c1ccc(-c2nc(-c3ccccc3)nc(-c3ccc4ccc5ccc6oc(-c7ccccc7)nc6c5c4c3)n2)cc1]

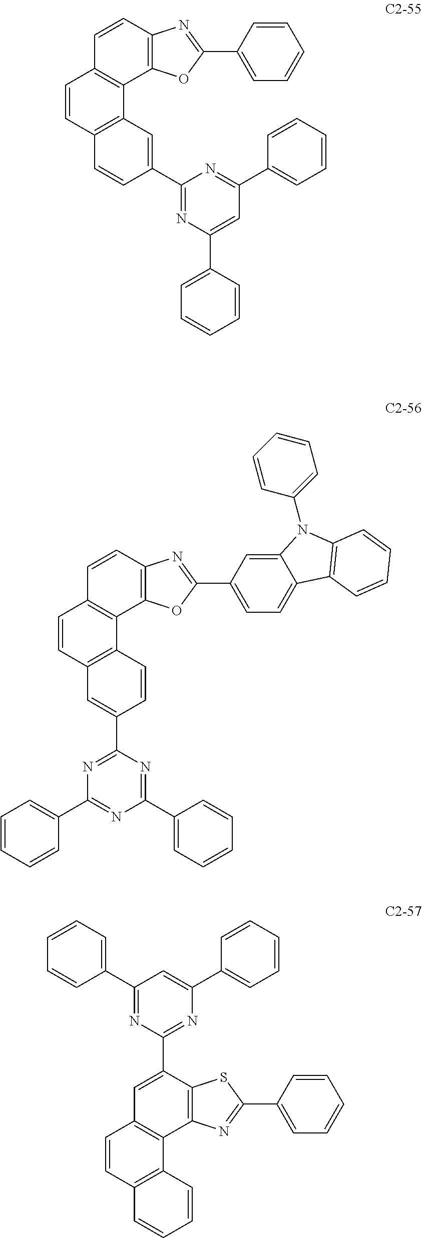 [SMILES: c1ccc(-c2cc(-c3ccccc3)nc(-c3ccc4ccc5ccc6nc(-c7ccccc7)oc6c5c4c3)n2)cc1 ; c1ccc(-c2nc(-c3ccccc3)nc(-c3ccc4c(ccc5ccc6nc(-c7ccc8c9ccccc9n(-c9ccccc9)c8c7)oc6c54)c3)n2)cc1 ; c1ccc(-c2cc(-c3ccccc3)nc(-c3cc4ccc5ccccc5c4c4nc(-c5ccccc5)sc34)n2)cc1]

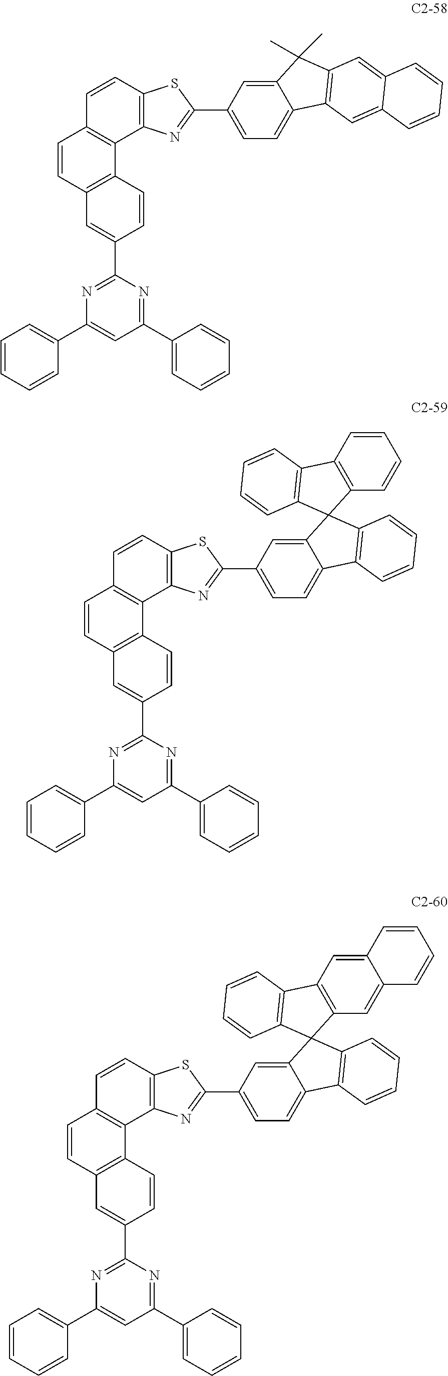 [SMILES: CC1(C)c2cc(-c3nc4c(ccc5ccc6cc(-c7nc(-c8ccccc8)cc(-c8ccccc8)n7)ccc6c54)s3)ccc2-c2cc3ccccc3cc21 ; c1ccc(-c2cc(-c3ccccc3)nc(-c3ccc4c(ccc5ccc6sc(-c7ccc8c(c7)C7(c9ccccc9-c9ccccc97)c7ccccc7-8)nc6c54)c3)n2)cc1 ; c1ccc(-c2cc(-c3ccccc3)nc(-c3ccc4c(ccc5ccc6sc(-c7ccc8c(c7)C7(c9ccccc9-8)c8ccccc8-c8cc9ccccc9cc87)nc6c54)c3)n2)cc1]

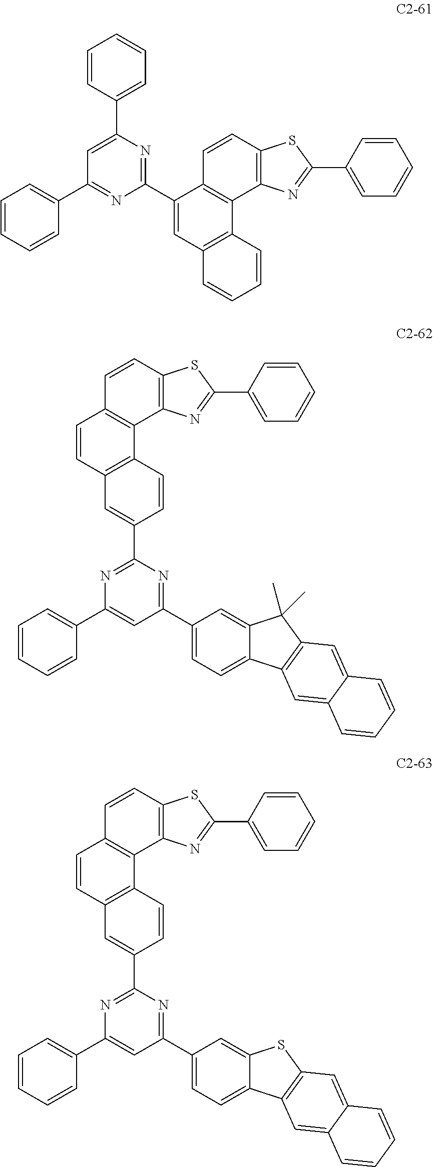 [SMILES: c1ccc(-c2cc(-c3ccccc3)nc(-c3cc4ccccc4c4c3ccc3sc(-c5ccccc5)nc34)n2)cc1 ; CC1(C)c2cc(-c3cc(-c4ccccc4)nc(-c4ccc5c(ccc6ccc7sc(-c8ccccc8)nc7c65)c4)n3)ccc2-c2cc3ccccc3cc21 ; c1ccc(-c2cc(-c3ccc4c(c3)sc3cc5ccccc5cc34)nc(-c3ccc4c(ccc5ccc6sc(-c7ccccc7)nc6c54)c3)n2)cc1]

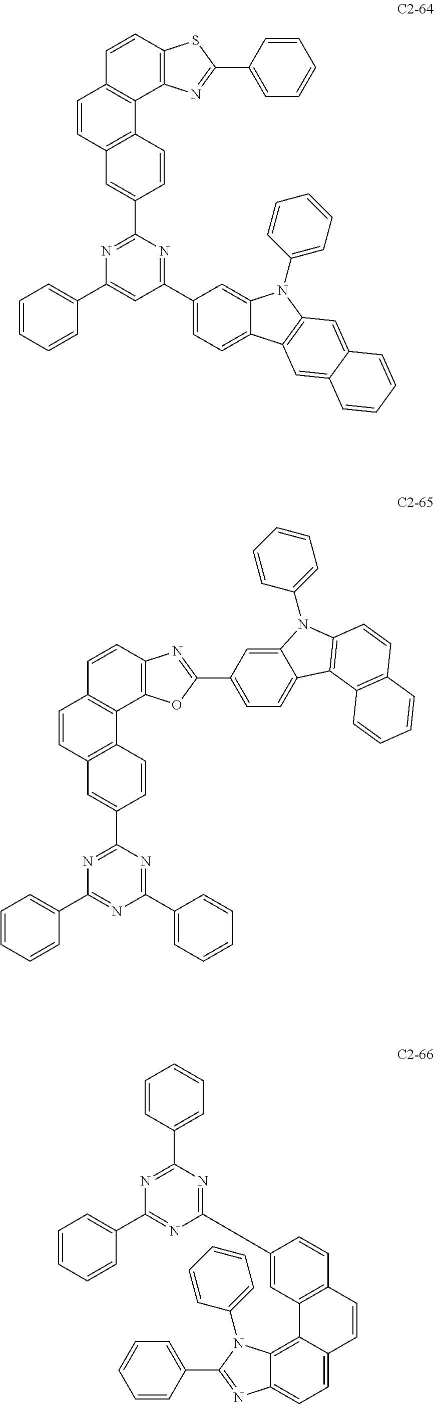 [SMILES: c1ccc(-c2cc(-c3ccc4c5cc6ccccc6cc5n(-c5ccccc5)c4c3)nc(-c3ccc4c(ccc5ccc6sc(-c7ccccc7)nc6c54)c3)n2)cc1 ; c1ccc(-c2nc(-c3ccccc3)nc(-c3ccc4c(ccc5ccc6nc(-c7ccc8c9c%10ccccc%10ccc9n(-c9ccccc9)c8c7)oc6c54)c3)n2)cc1 ; c1ccc(-c2nc(-c3ccccc3)nc(-c3ccc4ccc5ccc6nc(-c7ccccc7)n(-c7ccccc7)c6c5c4c3)n2)cc1]

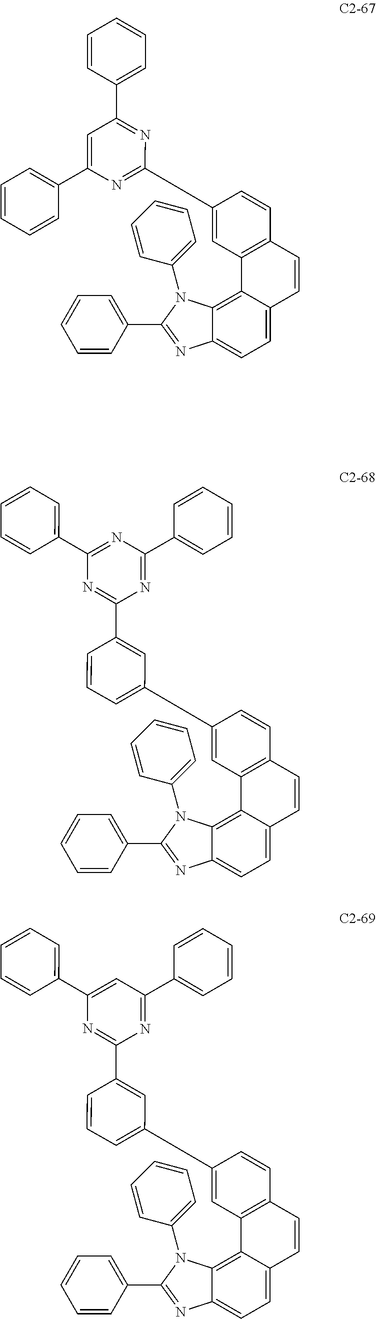 [SMILES: c1ccc(-c2cc(-c3ccccc3)nc(-c3ccc4ccc5ccc6nc(-c7ccccc7)n(-c7ccccc7)c6c5c4c3)n2)cc1 ; c1ccc(-c2nc(-c3ccccc3)nc(-c3cccc(-c4ccc5ccc6ccc7nc(-c8ccccc8)n(-c8ccccc8)c7c6c5c4)c3)n2)cc1 ; c1ccc(-c2cc(-c3ccccc3)nc(-c3cccc(-c4ccc5ccc6ccc7nc(-c8ccccc8)n(-c8ccccc8)c7c6c5c4)c3)n2)cc1]

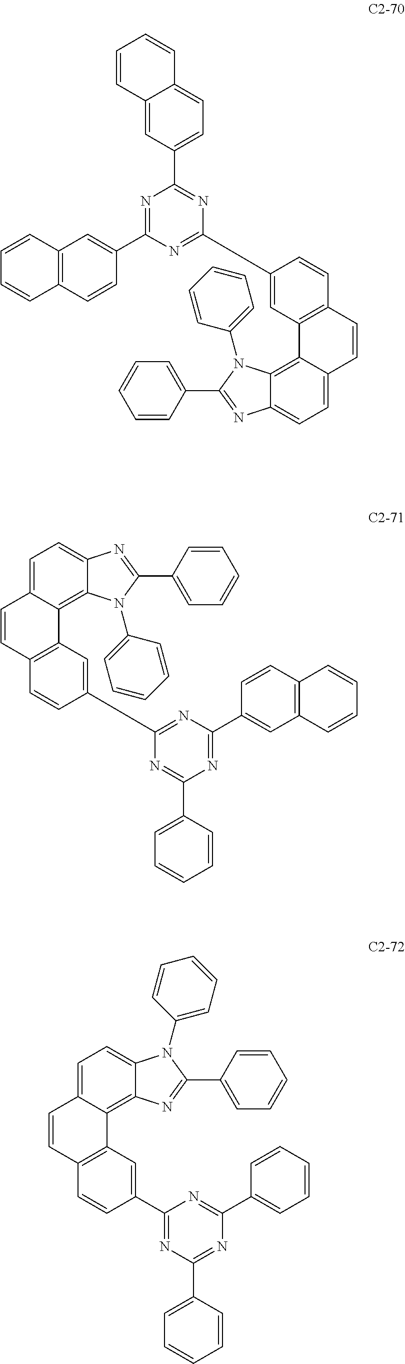 [SMILES: c1ccc(-c2nc3ccc4ccc5ccc(-c6nc(-c7ccc8ccccc8c7)nc(-c7ccc8ccccc8c7)n6)cc5c4c3n2-c2ccccc2)cc1 ; c1ccc(-c2nc(-c3ccc4ccccc4c3)nc(-c3ccc4ccc5ccc6nc(-c7ccccc7)n(-c7ccccc7)c6c5c4c3)n2)cc1 ; c1ccc(-c2nc(-c3ccccc3)nc(-c3ccc4ccc5ccc6c(nc(-c7ccccc7)n6-c6ccccc6)c5c4c3)n2)cc1]

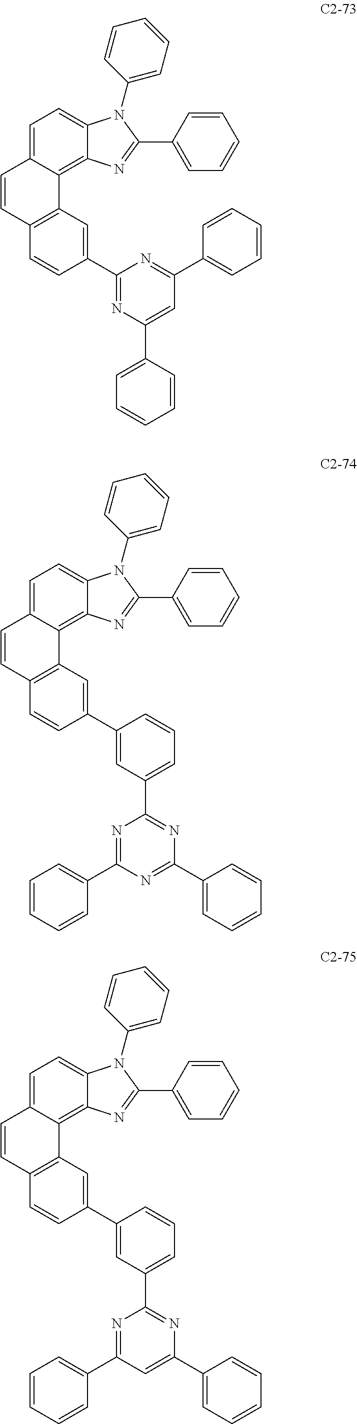 [SMILES: c1ccc(-c2cc(-c3ccccc3)nc(-c3ccc4ccc5ccc6c(nc(-c7ccccc7)n6-c6ccccc6)c5c4c3)n2)cc1 ; c1ccc(-c2nc(-c3ccccc3)nc(-c3cccc(-c4ccc5ccc6ccc7c(nc(-c8ccccc8)n7-c7ccccc7)c6c5c4)c3)n2)cc1 ; c1ccc(-c2cc(-c3ccccc3)nc(-c3cccc(-c4ccc5ccc6ccc7c(nc(-c8ccccc8)n7-c7ccccc7)c6c5c4)c3)n2)cc1]

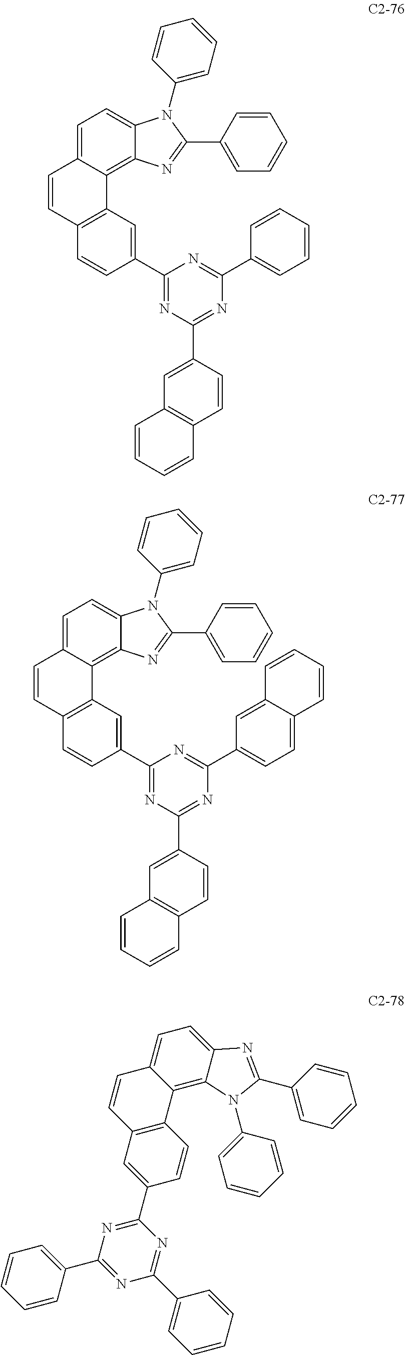

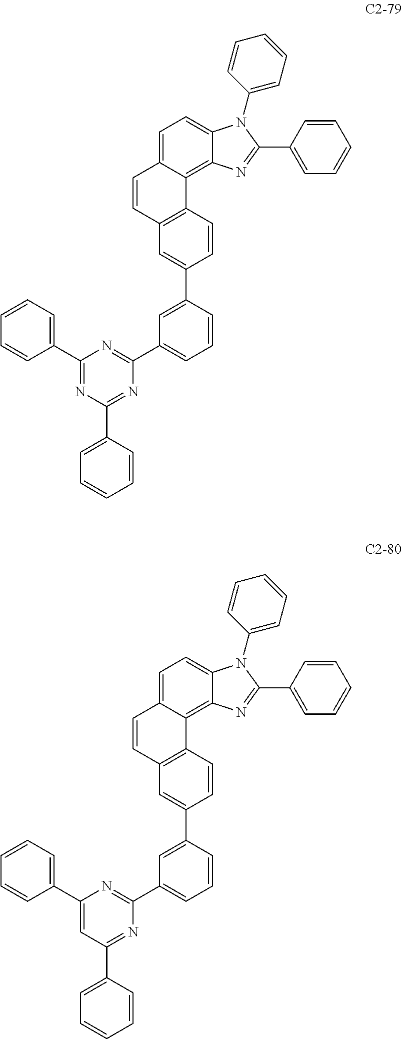 [SMILES: c1ccc(-c2nc(-c3ccccc3)nc(-c3cccc(-c4ccc5c(ccc6ccc7c(nc(-c8ccccc8)n7-c7ccccc7)c65)c4)c3)n2)cc1 ; c1ccc(-c2cc(-c3ccccc3)nc(-c3cccc(-c4ccc5c(ccc6ccc7c(nc(-c8ccccc8)n7-c7ccccc7)c65)c4)c3)n2)cc1]

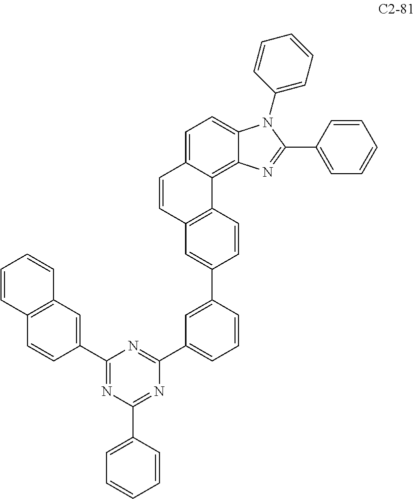 [SMILES: c1ccc(-c2nc(-c3cccc(-c4ccc5c(ccc6ccc7c(nc(-c8ccccc8)n7-c7ccccc7)c65)c4)c3)nc(-c3ccc4ccccc4c3)n2)cc1]

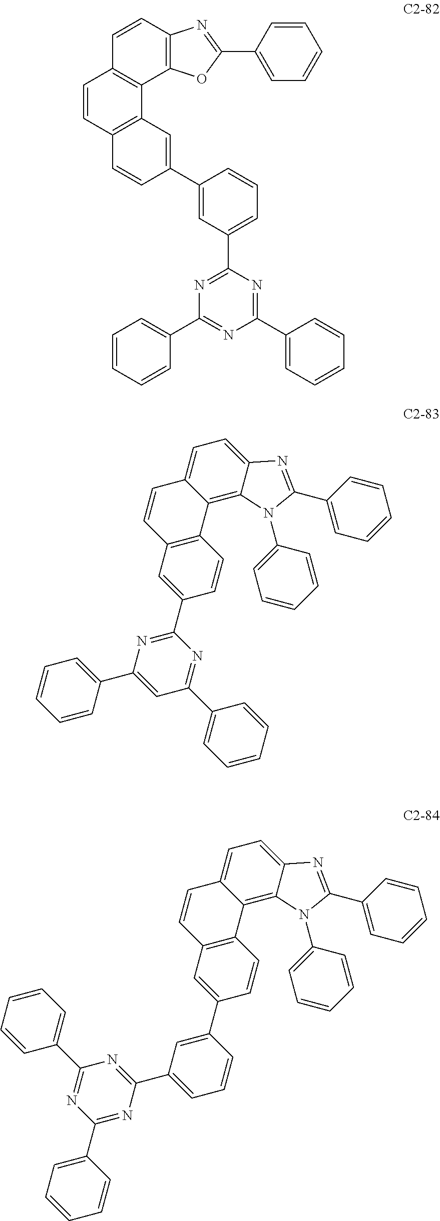 [SMILES: c1ccc(-c2nc(-c3ccccc3)nc(-c3cccc(-c4ccc5ccc6ccc7nc(-c8ccccc8)oc7c6c5c4)c3)n2)cc1 ; c1ccc(-c2cc(-c3ccccc3)nc(-c3ccc4c(ccc5ccc6nc(-c7ccccc7)n(-c7ccccc7)c6c54)c3)n2)cc1 ; c1ccc(-c2nc(-c3ccccc3)nc(-c3cccc(-c4ccc5c(ccc6ccc7nc(-c8ccccc8)n(-c8ccccc8)c7c65)c4)c3)n2)cc1]

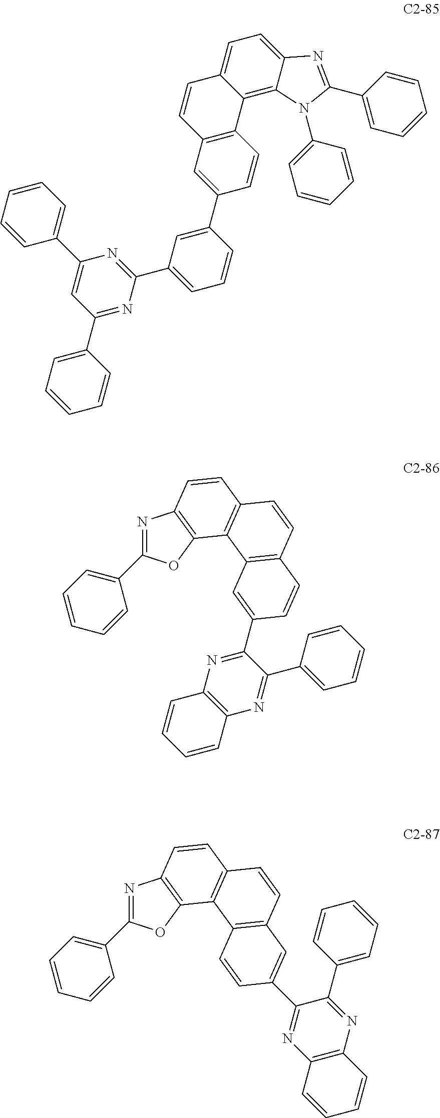 [SMILES: c1ccc(-c2cc(-c3ccccc3)nc(-c3cccc(-c4ccc5c(ccc6ccc7nc(-c8ccccc8)n(-c8ccccc8)c7c65)c4)c3)n2)cc1 ; c1ccc(-c2nc3ccc4ccc5ccc(-c6nc7ccccc7nc6-c6ccccc6)cc5c4c3o2)cc1 ; c1ccc(-c2nc3ccc4ccc5cc(-c6nc7ccccc7nc6-c6ccccc6)ccc5c4c3o2)cc1]

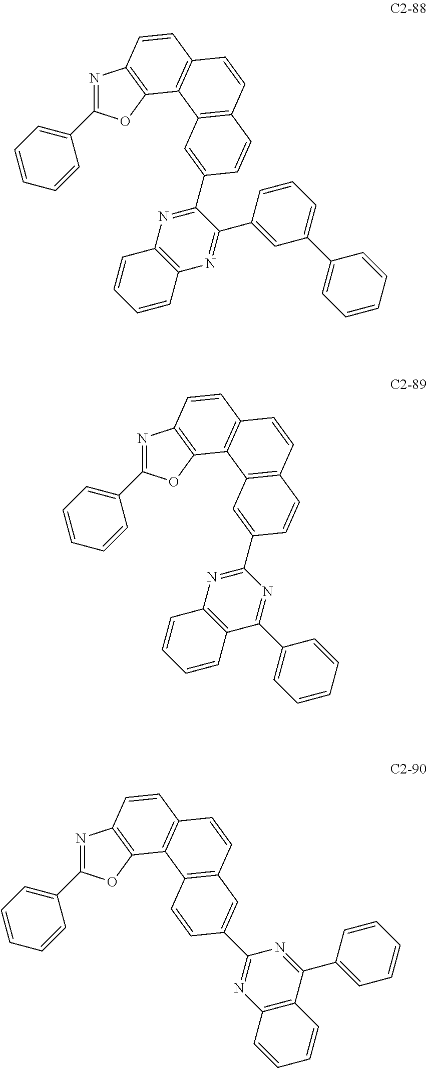 [SMILES: c1ccc(-c2cccc(-c3nc4ccccc4nc3-c3ccc4ccc5ccc6nc(-c7ccccc7)oc6c5c4c3)c2)cc1 ; c1ccc(-c2nc3ccc4ccc5ccc(-c6nc(-c7ccccc7)c7ccccc7n6)cc5c4c3o2)cc1 ; c1ccc(-c2nc3ccc4ccc5cc(-c6nc(-c7ccccc7)c7ccccc7n6)ccc5c4c3o2)cc1]

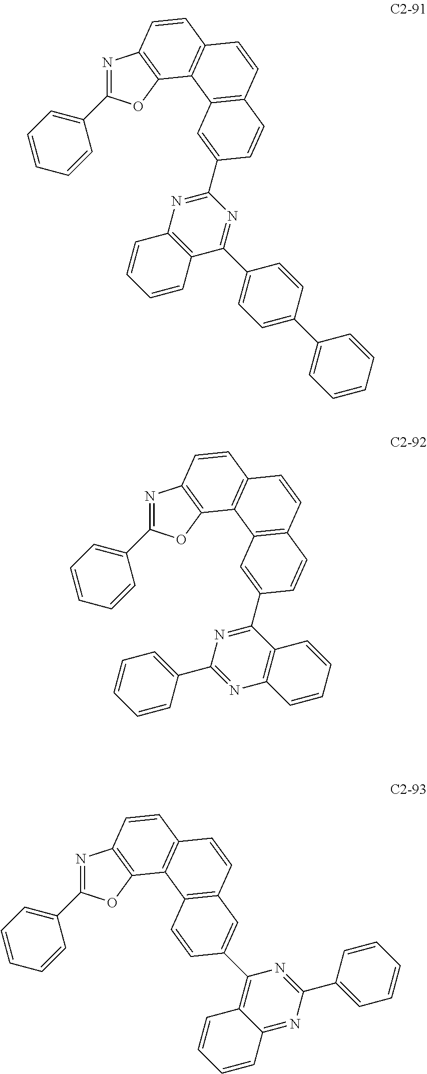 [SMILES: c1ccc(-c2ccc(-c3nc(-c4ccc5ccc6ccc7nc(-c8ccccc8)oc7c6c5c4)nc4ccccc34)cc2)cc1 ; c1ccc(-c2nc(-c3ccc4ccc5ccc6nc(-c7ccccc7)oc6c5c4c3)c3ccccc3n2)cc1 ; c1ccc(-c2nc(-c3ccc4c(ccc5ccc6nc(-c7ccccc7)oc6c54)c3)c3ccccc3n2)cc1]

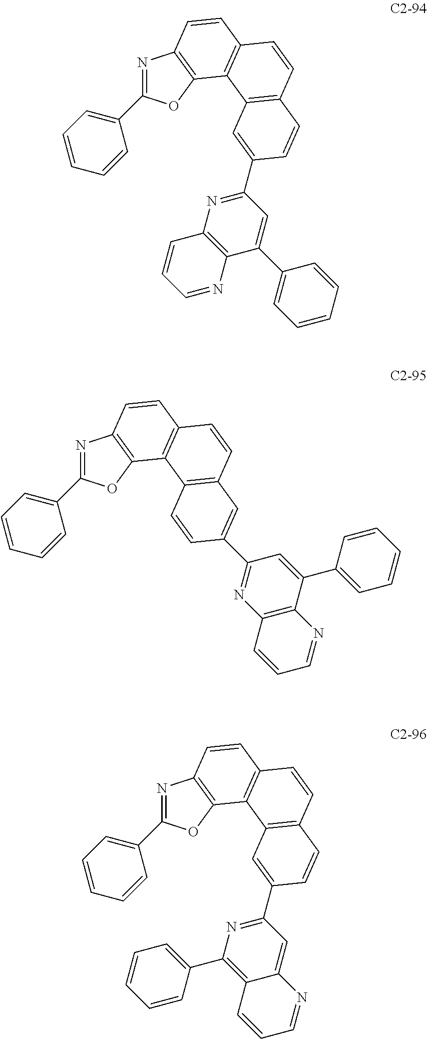 [SMILES: c1ccc(-c2nc3ccc4ccc5ccc(-c6cc(-c7ccccc7)c7ncccc7n6)cc5c4c3o2)cc1 ; c1ccc(-c2nc3ccc4ccc5cc(-c6cc(-c7ccccc7)c7ncccc7n6)ccc5c4c3o2)cc1 ; c1ccc(-c2nc3ccc4ccc5ccc(-c6cc7ncccc7c(-c7ccccc7)n6)cc5c4c3o2)cc1]

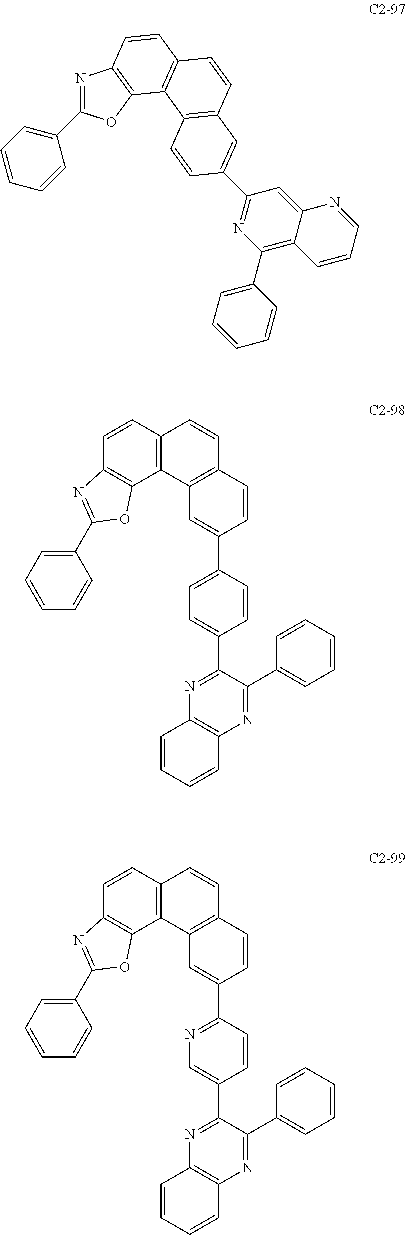 [SMILES: c1ccc(-c2nc3ccc4ccc5cc(-c6cc7ncccc7c(-c7ccccc7)n6)ccc5c4c3o2)cc1 ; c1ccc(-c2nc3ccc4ccc5ccc(-c6ccc(-c7nc8ccccc8nc7-c7ccccc7)cc6)cc5c4c3o2)cc1 ; c1ccc(-c2nc3ccc4ccc5ccc(-c6ccc(-c7nc8ccccc8nc7-c7ccccc7)cn6)cc5c4c3o2)cc1]

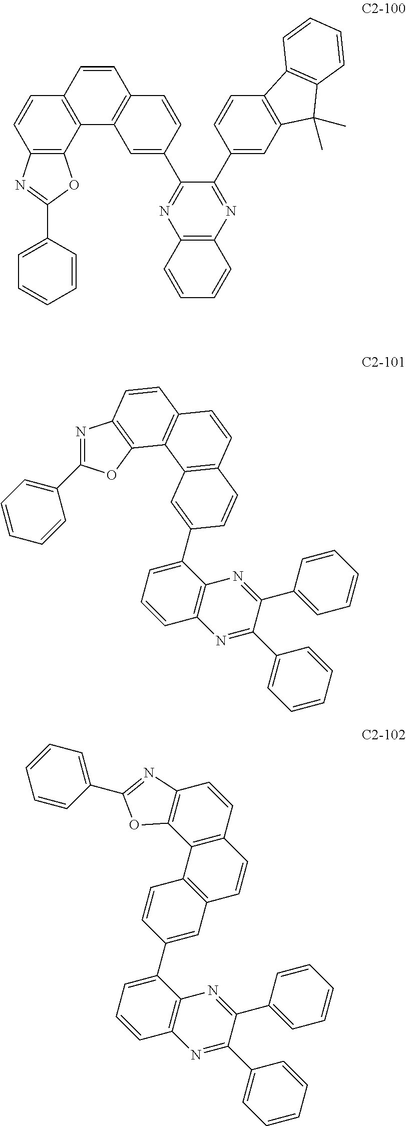 [SMILES: CC1(C)c2ccccc2-c2ccc(-c3nc4ccccc4nc3-c3ccc4ccc5ccc6nc(-c7ccccc7)oc6c5c4c3)cc21 ; c1ccc(-c2nc3ccc4ccc5ccc(-c6cccc7nc(-c8ccccc8)c(-c8ccccc8)nc67)cc5c4c3o2)cc1 ; c1ccc(-c2nc3ccc4ccc5cc(-c6cccc7nc(-c8ccccc8)c(-c8ccccc8)nc67)ccc5c4c3o2)cc1]

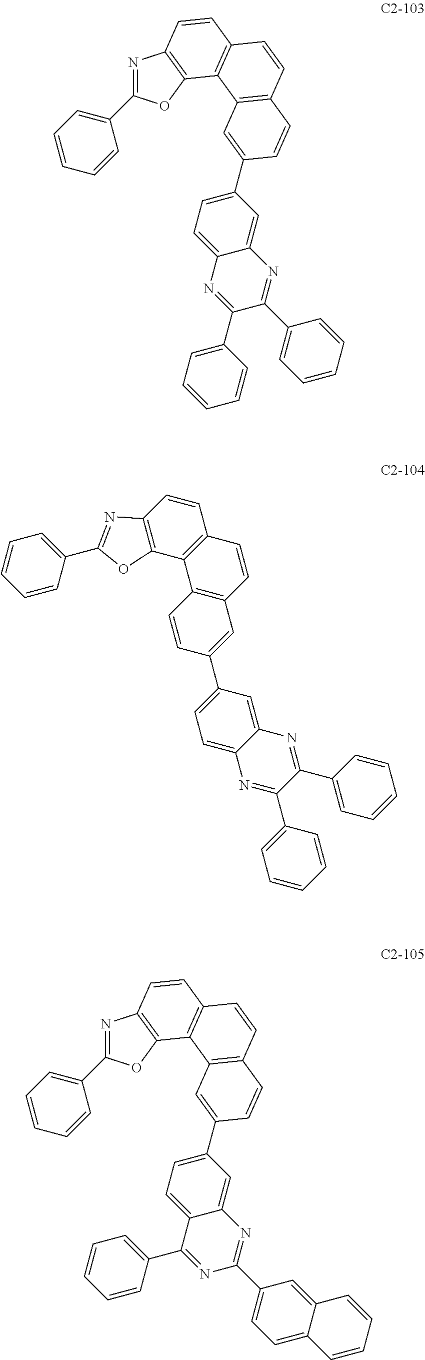 [SMILES: c1ccc(-c2nc3ccc4ccc5ccc(-c6ccc7nc(-c8ccccc8)c(-c8ccccc8)nc7c6)cc5c4c3o2)cc1 ; c1ccc(-c2nc3ccc4ccc5cc(-c6ccc7nc(-c8ccccc8)c(-c8ccccc8)nc7c6)ccc5c4c3o2)cc1 ; c1ccc(-c2nc3ccc4ccc5ccc(-c6ccc7c(-c8ccccc8)nc(-c8ccc9ccccc9c8)nc7c6)cc5c4c3o2)cc1]

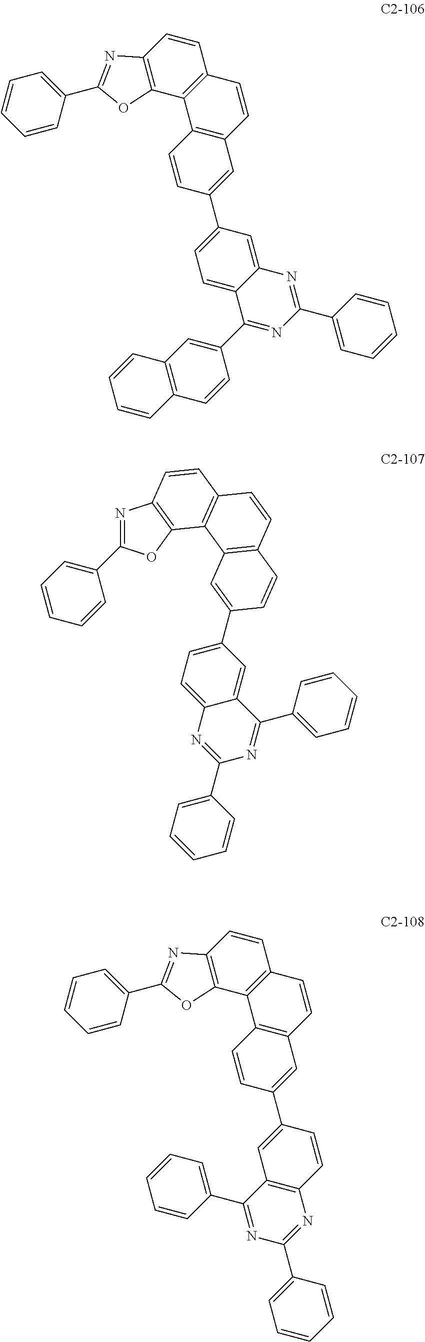 [SMILES: c1ccc(-c2nc(-c3ccc4ccccc4c3)c3ccc(-c4ccc5c(ccc6ccc7nc(-c8ccccc8)oc7c65)c4)cc3n2)cc1 ; c1ccc(-c2nc(-c3ccccc3)c3cc(-c4ccc5ccc6ccc7nc(-c8ccccc8)oc7c6c5c4)ccc3n2)cc1 ; c1ccc(-c2nc(-c3ccccc3)c3cc(-c4ccc5c(ccc6ccc7nc(-c8ccccc8)oc7c65)c4)ccc3n2)cc1]

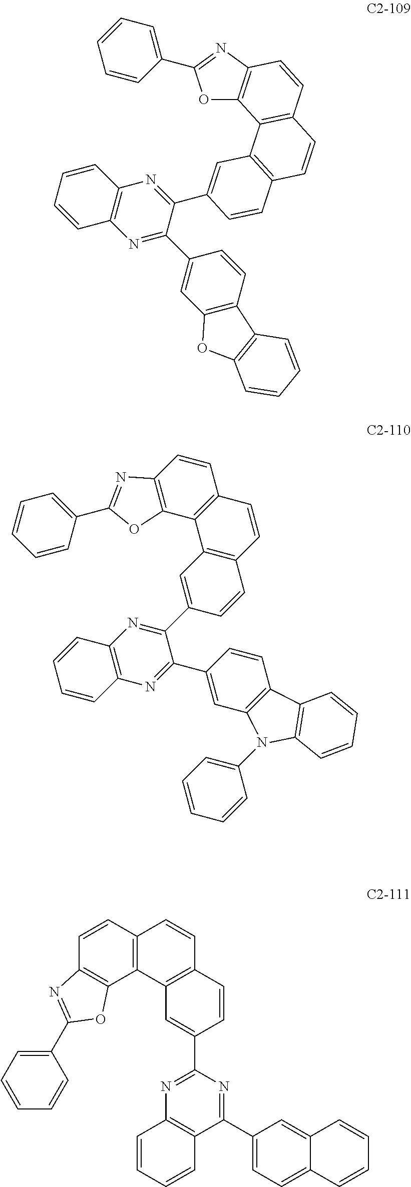 [SMILES: c1ccc(-c2nc3ccc4ccc5ccc(-c6nc7ccccc7nc6-c6ccc7c(c6)oc6ccccc67)cc5c4c3o2)cc1 ; c1ccc(-c2nc3ccc4ccc5ccc(-c6nc7ccccc7nc6-c6ccc7c8ccccc8n(-c8ccccc8)c7c6)cc5c4c3o2)cc1 ; c1ccc(-c2nc3ccc4ccc5ccc(-c6nc(-c7ccc8ccccc8c7)c7ccccc7n6)cc5c4c3o2)cc1]

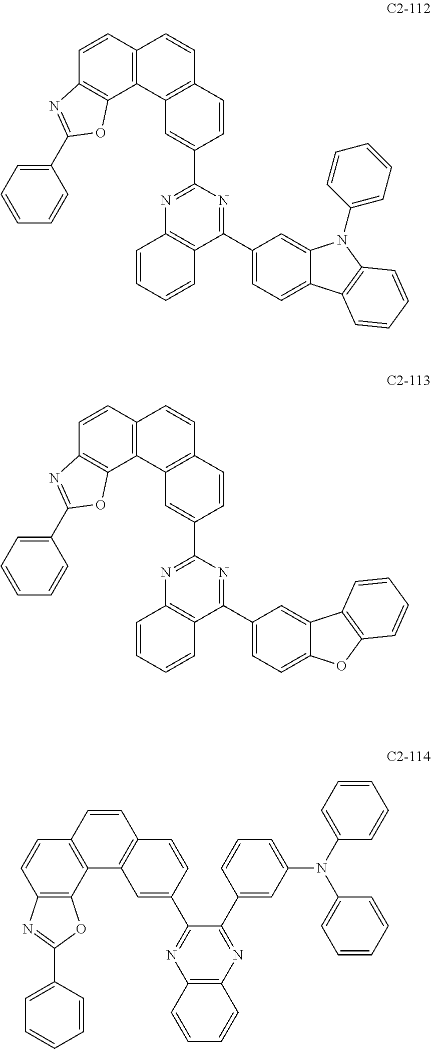 [SMILES: c1ccc(-c2nc3ccc4ccc5ccc(-c6nc(-c7ccc8c9ccccc9n(-c9ccccc9)c8c7)c7ccccc7n6)cc5c4c3o2)cc1 ; c1ccc(-c2nc3ccc4ccc5ccc(-c6nc(-c7ccc8oc9ccccc9c8c7)c7ccccc7n6)cc5c4c3o2)cc1 ; c1ccc(-c2nc3ccc4ccc5ccc(-c6nc7ccccc7nc6-c6cccc(N(c7ccccc7)c7ccccc7)c6)cc5c4c3o2)cc1]

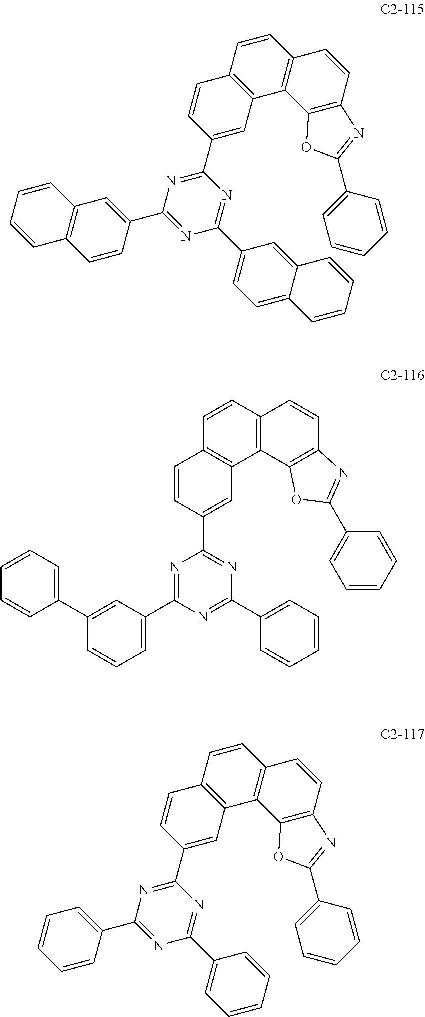 [SMILES: c1ccc(-c2nc3ccc4ccc5ccc(-c6nc(-c7ccc8ccccc8c7)nc(-c7ccc8ccccc8c7)n6)cc5c4c3o2)cc1 ; c1ccc(-c2cccc(-c3nc(-c4ccccc4)nc(-c4ccc5ccc6ccc7nc(-c8ccccc8)oc7c6c5c4)n3)c2)cc1 ; c1ccc(-c2nc(-c3ccccc3)nc(-c3ccc4ccc5ccc6nc(-c7ccccc7)oc6c5c4c3)n2)cc1]

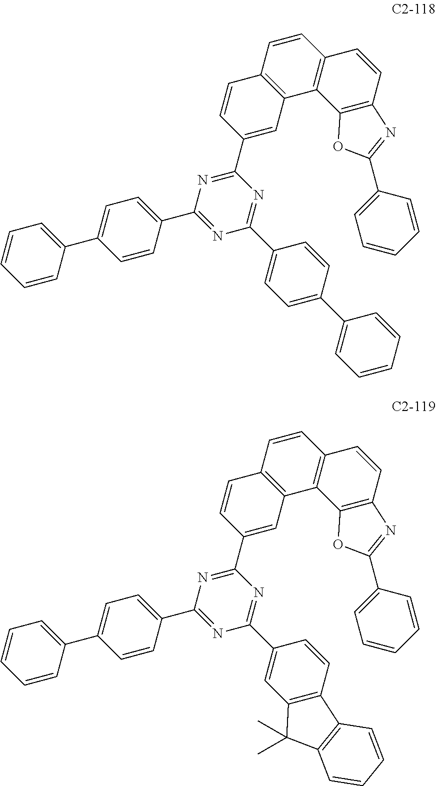 [SMILES: c1ccc(-c2ccc(-c3nc(-c4ccc(-c5ccccc5)cc4)nc(-c4ccc5ccc6ccc7nc(-c8ccccc8)oc7c6c5c4)n3)cc2)cc1 ; CC1(C)c2ccccc2-c2ccc(-c3nc(-c4ccc(-c5ccccc5)cc4)nc(-c4ccc5ccc6ccc7nc(-c8ccccc8)oc7c6c5c4)n3)cc21]

9. An organic electroluminescent device comprising an anode, a cathode, and at least one light-emitting layer between the anode and the cathode, wherein the at least one layer of the light-emitting layers comprises the plurality of host materials according to claim 1.
